(19) United States
(12) Patent Application Publication
(10) Pub. No.: US 2003/0215877 A1
(43) Pub. Date: Nov. 20, 2003

(54) Directed protein docking algorithm

(75) Inventors: John J. Love, La Mesa, CA (US); Stephen L. Mayo, Pasadena, CA (US)

Correspondence Address: ROPES & GRAY LLP, ONE INTERNATIONAL PLACE, BOSTON, MA, 02110-2624, US

(73) Assignee: California Institute of Technology, Pasadena, CA

(21) Appl. No.: 10/407,480

(22) Filed: Apr. 4, 2003

Related U.S. Application Data
(60) Provisional application No. 60/370,167, filed on Apr. 4, 2002.

(51) Int. Cl. G01N 33/53; G06G 7/48; G06G 7/58; G06F 19/00; G01N 33/48; G01N 33/50; C12P 21/02; C12N 5/06

(52) U.S. Cl. 435/7.1; 435/69.1; 435/320.1; 435/325; 702/19; 703/11

(57) ABSTRACT

The instant invention provides methods and computational tools for designing interaction between molecules based on their three-dimensional atomic coordinates. In a preferred embodiment, the method can be used to design protein-protein interactions based on their three-dimensional structure. In one embodiment, the method of the instant invention includes a first step of docking interacting molecules based on their surface geometric fit by quantitative correlation techniques, followed by a second step of optimizing the resulting interacting surface by altering interface side-chains, such that the interfacial side-chains are repacked in a manner analogous to the cores of well-folded proteins. The method can be used in numerous applications, including redesigning interaction interfaces between known protein-protein, protein-polynucleotide, protein-carbohydrates (such as polysaccharide), protein-lipid (or steroid), enzyme-inhibitor, or antibody-target epitope pairs, or rational design of more potent drug molecules.

### REFERENCE TO RELATED APPLICATION

This application claims the benefit under 35 U.S.C. 119(e) of U.S. Provisional Application No. 60/370,167, filed on Apr. 4, 2002, the entire content of which is incorporated herein by reference.

### BACKGROUND OF THE INVENTION

Protein-protein interactions are responsible for a wide variety of important biological phenomena from immune recognition to transcription initiation and signal transduction. While many methods exist for determining whether two proteins interact, few techniques address the need to design one molecule that can interact with another molecule, especially proteins that can interact specifically with a target protein.

Previously developed, non-computational methods for generating novel mutations in proteins for binding to a specific target protein include, for example, phage-display, yeast and bacterial two-hybrid screens, ribosome display and mRNA covalent attachment methods. However, one major limitation to these methods is the sequence complexity accessible to these methods. The highest reported sequence complexity assessable to these other methods is approximately 10¹⁵ (the mRNA covalent attachment method). Therefore, saturation mutagenesis (i.e. substituting for all 20 amino acids) of 10 positions (20¹⁰, or about 1×10¹³ potential sequences) is theoretically possible if using the best available experimental (non-computational) methods. However, these traditional experimental methods are frequently limited by the ability of cells to actually produce these many possible mutations, or the ability to exhaustively screen all produced mutations, or both. Thus there is a need to develop novel methods that will enable the screening of larger sequence spaces (a collection of all screenable sequences).

On the other hand, in the field of modeling/predicting native protein interaction using computational methods, the general strategy for simulating protein-protein docking involves matching shape complementarity (for recent reviews, see Janin, 1995, Prog. Phys. Mol. Biol. 64: 145; Shoichet & Kuntz, Chem. Bio. 3: 151). Some approaches focus specifically on matching surfaces (e.g. see Jiang & Kim, 1991, J. Mol. Biol. 219: 79; Katchalski-Katzir et al., 1992, PNAS USA 89: 2195; Walls & Stemberg, 1992, J. Mol. Biol. 228: 277; Helmer-Citterich & Tramontano, 1994, J. Mol. Biol. 235: 1021). Others enhance the search for geometric complimentarity by matching the position of surface spheres and surface normals (e.g. see Kuntz et al., 1982, J. Mol. Biol. 161: 269; Shoichet & Kuntz, 1991, supra; Norel et al., 1995, J. Mol. Biol. 252: 263). Shape complementarity is measured by a variety of scoring functions, some of which aim to model the hydrophobic effect during association from the change in solvent-accessible surface area of molecular surface area (e.g. see Cherfils et al., 1991, Proteins 11: 271). Several algorithms employ a simplified scheme to estimate electrostatic interactions (e.g. see Jian & Kim, 1991, supra; Walls & Sternberg, 1992, supra). In general the algorithms yield a limited set of favorable complexes, one or a few of which are close (typically 1 to 3 Å RMS) to the native structure. Recognizing this, several groups have additionally focused on screening the correct solution from the false positives by modeling the hydrophobic effect, electrostatic interactions and desolvation (Gilson & Honig, 1988, Nature 330: 84; Vakser & Aflalo, 1994, Proteins 20: 320; Jackson & Sternberg, 1995, J. Mol. Biol. 250: 258; Weng et al., 1996, Protein Sci. 5: 614). Most of the above studies have focused on rigid body docking. Recently, however, Monte Carlo simulations have been used to refine flexible side-chain positions after rigid body docking (Totrov & Abagyan, 1994, Nature Struct. Biol. 1: 259).

The association of proteins with their ligands involves intricate inter- and intramolecular interactions, solvation effects, and conformational changes. In view of such complexity, a comprehensive and efficient approach for predicting the formation of protein-ligand complexes from the structure of their free components is desirable. With some assumptions, such predictions become feasible, and several attempts based on energy minimization have been reasonably successful (Wodak and Janin, 1978, J. Mol. Biol. 124: 323; Yue, 1990, Protein Eng. 4: 177). Another simplifying approach that could alleviate some of these difficulties is based on geometric considerations.

The three-dimensional (3D) structures of most protein complexes reveal a close geometric match between those parts of the respective surfaces of the protein and the ligand that are in contact. Indeed, the shape and other physical characteristics of the surfaces largely determine the nature of the specific molecular interactions in the complex. Furthermore, in many cases the 3D structure of the components in the complex closely resembles that of the molecules in their free, native state. Geometric matching thus plays an important role in determining the structure of a complex.

Several investigators have exploited a geometric approach to find shape complementarity between a given protein and its ligand (Greer and Bush, 1978, P.N.A.S. USA 75: 303; Wang, 1991, J. Comp. Chem. 12: 746). They considered geometric match between molecular surfaces as a fundamental condition for the formation of a specific complex and pointed out the advantages of the geometric approach (Connolly, 1986, Biopolymers 25: 1229). In this approach, which treats proteins as rigid bodies, the complementarity between surfaces is estimated. Furthermore, the geometric analysis could serve as the foundation for a more complete approach including energy considerations. However, the methods heretofore developed for analyzing geometric matching do not seem to simultaneously fulfill the requirements for generality, accuracy, reliability, and reasonable computation time.

Katchalski-Katzir et al. (P.N.A.S. USA 89: 2195, 1992) present a geometry-based algorithm for predicting the structure of a possible complex between molecules of known structures. This relatively simple and straightforward algorithm relies on the well-established correlation and Fourier transformation techniques used in the field of pattern recognition. The algorithm requires only that the 3D structure of the molecules under consideration be known or readily obtainable. Moreover, it provides quantitative data related to the quality of the contact between the molecules. The algorithm was tested and validated in the analysis of several complexes whose structures were known: the α-β hemoglobin dimer, tRNA synthetase-tyrosinyl adenylate, aspartic proteinase-peptide inhibitor, and trypsin-trypsin inhibitor. The correct relative position of the molecules within these complexes were successfully predicted. Gabb et al. (J. Mol. Biol. 272: 106-120, 1997) considers not only geometric shapes of interacting proteins, but also non-geometric factors such as electrostatics and biochemical information.

### SUMMARY OF THE INVENTION

One aspect of the invention provides a method for modifying a candidate polypeptide sequence to alter interaction with a target biopolymer, comprising: (a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) identifying, by surface-to-surface geometric fitting, a model of a complex between said target biopolymer model and said candidate polypeptide model that has at least a predefined degree of surface shape complementarity; (c) identifying amino acid residues in said candidate polypeptide with unfavorable interactions with said target biopolymer in said complex as varying residues; (d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

In one embodiment, said atomic coordinate model of said candidate polypeptide includes coordinates for only backbone atoms but not C_{β} atoms of said reference amino acid sequence.

In one embodiment, said atomic coordinate model of said candidate polypeptide and said atomic coordinate model of said target biopolymer are obtained from known crystallographic or NMR structures.

Alternatively, said atomic coordinate model of said candidate polypeptide and said atomic coordinate model of said target biopolymer are established by homology modeling based on a known crystallographic or NMR structure of a homolog of said target biopolymer or a homolog of said candidate polypeptide. Preferably, said homolog is at least about 70% identical to said candidate polypeptide in the binding region; or at least about 70% identical to said target biopolymer, wherein said target biopolymer is a polypeptide.

In one embodiment, said target biopolymer is a lipid, a vitamin co-factor, or a steroid.

In one embodiment, said target biopolymer is a protein, a polynucleotide, or a polysaccharide.

In one embodiment, said target biopolymer is a protein, and wherein said docking surface is an atomic coordinate model of said target protein, which model includes coordinates for at least backbone atoms of exposed surface residues. In a preferred embodiment, said target protein model additionally include coordinates for C_{β} atoms of exposed surface residues. In another preferred embodiment, said target protein model additionally include coordinates for more than C_{β} atoms of exposed surface residues. In yet another preferred embodiment, said target protein model additionally include coordinates for at least backbone atoms of non-surface residues.

In one embodiment, said surface-to-surface geometric fitting is identified in step (b) by: (A) computationally projecting said atomic coordinate model of said candidate polypeptide and said target biopolymer onto a three-dimensional grid, and fixing the atomic coordinate model of said target biopolymer in a pre-defined target orientation; (B) assessing intermolecular surface shape complementarity between said candidate polypeptide and said target biopolymer as a function of their relative translational and rotational positions, by rotating and translating the atomic coordinate model of said candidate polypeptide; (C) identifying the optimal atomic coordinate model associated with the best intermolecular surface shape complementarity; and, (D) combining the optimal atomic coordinate models of the docked said candidate polypeptide and said target biopolymer as the atomic coordinate model of said complex.

In one embodiment, step (c) is effected by: (A) classifying residues of said candidate polypeptide as core, boundary, or surface residues, first in the context of the undocked form and then in the context of said complex; and, (B) identifying residues which either change classification upon complex formation, or are in close proximity to form favorable intermolecular interactions as said varying residues. In a preferred embodiment, said target biopolymer is a protein.

In one embodiment, step (d) is effected by: (A) providing the coordinates for a plurality of potential rotamers resulting from varying torsional angles for side-chains of each of said varying residues identified in (c), wherein said plurality of potential rotamers for at least one of said varying residues have rotamers selected from each of at least two different amino acid side-chains; and (B) modeling interactions of each of said rotamers with all or part of the remaining structure of said complex to generate a set of globally optimized protein sequences.

In a preferred embodiment, said three-dimensional grid comprises N×N×N nodes. For example, Ncan be 32, 64, 128, 256, 512, 1024, or any number in between.

In another preferred embodiment, the size of said grid is the sum of the radii of said candidate polypeptide and said target biopolymer plus 0.5, 1, 2, or 5 Å.

In another embodiment, the size of said grid is the sum of the radii of said candidate polypeptide and a potential candidate-polypeptide-binding region of said target biopolymer plus 0.5, 1, 2, or 5 Å.

In yet another embodiment, said surface-to-surface geometric fitting is identified by a geometric recognition algorithm (GRA). In addition, said GRA may further incorporates a Fourier Correlation Algorithm (FCA). Said FCA may comprise discrete fast Fourier transformation (DFT) of said candidate polypeptide and said target biopolymer.

In any of the above embodiments, the method may further comprise measuring electrostatic complementarity by Fourier correlation; and/or distance filtering; and/or local refinement of predicted geometries.

In any of the above embodiments, the method is repeated more than once with successively more fine-tuned parameters for assessing intermolecular surface-to-surface geometric fitting.

In any of the above embodiments, the method may further comprise one or more of: measuring electrostatic complementarity by Fourier correlation, distance filtering, or local refinement of predicted geometries.

In one embodiment, said plurality of potential rotamers for said varying residues are from a backbone-dependent rotamer library.

In one embodiment, said torsional angles for side-chains of each of said varying residues are changed by varying both the χ1 and χ2 torsional angles by ±20 degrees, in increment of 5 degrees, from the values of said varying residues in the context of the undocked candidate polypeptide.

In one embodiment, the method further comprise a Dead-End Elimination (DEE) computation in step (d). The DEE computation may be selected from original DEE or Goldstein DEE. In a related embodiment, the calculation method further includes the use of at least one, two, three, or four scoring functions. Such scoring function may be selected from: van der Waals potential scoring function, hydrogen bond potential scoring function, atomic solvation scoring function, electrostatic scoring function or secondary structure propensity scoring function.

In certain embodiments, said atomic solvation scoring function includes a scaling factor that compensates for over-counting.

In one embodiment, the method further comprise generating a rank ordered list of additional optimal sequences from said globally optimal protein sequence. For example, said generating may include the use of a Monte Carlo search.

In one embodiment, the method further comprise testing some or all of said protein sequences from said ordered list to produce potential energy test results. In a preferred embodiment, the method further comprises analyzing the correspondence between said potential energy test results and theoretical potential energy data.

In one embodiment, said varying residue identified in step (c) are residues re-classified as core residues upon complex formation, and wherein said plurality of potential rotamers for said varying residues have rotamers selected from each of at least two different hydrophobic amino acid side-chains. For example, said at least two hydrophobic amino acids are selected from: alanine, valine, isoleucine, leucine, phenylalanine, tyrosine, tryptophan, or methionine.

In one embodiment, said varying residue identified in step (c) are residues re-classified from surface to boundary residues upon complex formation, and wherein said plurality of potential rotamers for said varying residues have rotamers selected from each of at least two different hydrophilic amino acid side-chains. For example, said at least two hydrophilic amino acids are selected from: alanine, serine, threonine, aspartic acid, asparagine, glutamine, glutamic acid, arginine, lysine or histidine.

In one embodiment, said varying residue identified in step (c) are residues re-classified as boundary residues upon complex formation, and wherein said plurality of potential rotamers for said varying residues have rotamers selected from each of at least two different amino acid side-chains selected from: alanine, serine, threonine, aspartic acid, asparagine, glutamine, glutamic acid, arginine, lysine histidine, valine, isoleucine, leucine, phenylalanine, tyrosine, tryptophan, or methionine.

In one embodiment, the method further comprises generating said target biopolymer, and one or more modified versions of said candidate polypeptide with said mutations of said varying residues that form more favorable interactions with said target biopolymer model, and assessing the degree of complex formation. For example, said degree of complex formation can be assessed in vitro or in vivo, or both.

In one embodiment, the method further comprises verifying, by solving the three-dimensional structure(s) of, one or more modified versions of said candidate polypeptide with said mutations of said varying residues that form more favorable interactions with said target biopolymer model.

In one embodiment, said candidate polypeptide is an antibody or functional fragment thereof.

In one embodiment, said target biopolymer is an enzyme, and said candidate polypeptide is an inhibitor of said enzyme.

In one embodiment, said target biopolymer is a target protein, wherein step (c) further includes identifying amino acid residues in said target protein with unfavorable interactions with said candidate polypeptide in said complex as varying residues, and wherein step (d) is additionally effected by identifying mutations of said varying residues of said target protein that form more favorable interactions with said candidate polypeptide. For example, said target protein and said candidate polypeptide are identical.

It is contemplated that the above embodiments, especially embodiments directed to independent features or different aspects of the inventions, can be combined at any level of details when appropriate.

Another aspect of the invention provides a complex comprising a target biopolymer and a redesigned candidate polypeptide generated by any suitable method described above.

Another aspect of the invention provides a nucleic acid sequence encoding a target polypeptide and a nucleic acid sequence encoding a redesigned candidate polypeptide described above.

Another aspect of the invention provides an expression vector comprising the nucleic acid sequences described above.

Another aspect of the invention provides a host cell comprising the nucleic acid sequences described above.

Another aspect of the invention provides an apparatus for redesigning a candidate polypeptide sequence to alter interaction with a target biopolymer, said apparatus comprising: (a) means for providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) means for identifying, by surface-to-surface geometric fitting, a model of a complex between said target biopolymer model and said candidate polypeptide model that has at least a predefined degree of surface shape complementarity; (c) means for identifying amino acid residues in said candidate polypeptide with unfavorable interactions with said target biopolymer in said complex as varying residues; (d) means for generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

The apparatus may further include any of the similar features and combinations thereof, as described above for the corresponding claimed method.

Another aspect of the invention provides a computer system for use in redesigning a candidate polypeptide sequence to alter interaction with a target biopolymer, said computer system comprising computer instructions for: (a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) identifying, by surface-to-surface geometric fitting, a model of a complex between said target biopolymer model and said candidate polypeptide model that has at least a predefined degree of surface shape complementarity; (c) identifying amino acid residues in said candidate polypeptide with unfavorable interactions with said target biopolymer in said complex as varying residues; (d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

The computer system may further include any of the similar features and combinations thereof, as described above for the corresponding claimed method.

Another aspect of the invention provides a computer-readable medium storing a computer program executable by a plurality of server computers, the computer program comprising computer instructions for: (a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) identifying, by surface-to-surface geometric fitting, a model of a complex between said target biopolymer model and said candidate polypeptide model that has at least a predefined degree of surface shape complementarity; (c) identifying amino acid residues in said candidate polypeptide with unfavorable interactions with said target biopolymer in said complex as varying residues; (d) generating one or more model(s) of said complex in which said candidate polypeptide model includes atomic coordinates of more than the C_{β}0 atoms of said varying residue side-chains, and identifying mutations of said varying residues that form more favorable interactions with said target biopolymer model.

The computer-readable medium may further include any of the similar features and combinations thereof, as described above for the corresponding claimed method.

Another aspect of the invention provides a computer data signal embodied in a carrier wave, comprising computer instructions for: (a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) identifying, by surface-to-surface geometric fitting, a model of a complex between, said target biopolymer model and said candidate polypeptide model that has at least a predefined degree of surface shape complementarity; (c) identifying amino acid residues in said candidate polypeptide with unfavorable interactions with said target biopolymer in said complex as varying residues; (d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

The computer data signal embodied in a carrier wave may further include any of the similar features and combinations thereof, as described above for the corresponding claimed method.

Another aspect of the invention provides an apparatus comprising a computer readable storage medium having instructions stored thereon for: (a) accessing a datafile representative of (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer; (b) accessing a datafile representative of the atomic coordinates for a plurality of different rotamers of amino acids resulting from varying torsional angles; (c) a set of modeling routines for: (1) identifying surface-to-surface geometric fitting by docking said candidate polypeptide and said target biopolymer to form a complex with a predefined degree of surface shape complementarity between said candidate polypeptide and said target biopolymer; (2) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

The apparatus may further include any of the similar features and combinations thereof, as described above for the corresponding claimed method.

Another embodiment of the invention provides a method for conducting a biotechnology business comprising: (1) redesigning, according to the method of claim 1, a candidate polypeptide sequence to alter interaction with a target biopolymer; (2) producing said candidate polypeptide.

In one embodiment, the business method further comprising the step of providing a packaged pharmaceutical including said candidate polypeptide and/or said target biopolymer, and instructions and/or a label describing how to administer said redesigned candidate polypeptide.

Another aspect of the invention provides a method for inhibiting the binding of a candidate polypeptide to a target biopolymer, comprising: (a) redesigning, using the method of claim 1, a set of globally optimized complexes comprising a redesigned candidate polypeptide and said target biopolymer; (b) obtaining an inhibitory polypeptide sequence comprising the interfacial residue sequences of said redesigned candidate polypeptide; (c) providing said inhibitory polypeptide sequence to a mixture containing said candidate polypeptide and said target biopolymer, thereby inhibiting the binding of said candidate polypeptide to said target biopolymer.

Another aspect of the invention provides a method for redesigning a candidate molecule for binding to a target polypeptide sequence, comprising: (a) providing atomic coordinates for at least the backbone sequences of said target polypeptide and atomic coordinates for said candidate molecule, (b) docking, using said atomic coordinates of (a), said candidate molecule to said target polypeptide to form a pseudo complex with the best intermolecular surface complementarity; (c) modeling interfacial side-chains or groups of atoms of said candidate molecule to generate a set of globally optimized pseudo complexes, thereby redesigning said candidate molecule for binding to said target polypeptide.

Another aspect of the invention provides a method for redesigning a candidate polypeptide for binding to a target molecule sequence, comprising: (a) providing atomic coordinates for at least the backbone sequences of said candidate polypeptide and atomic coordinates for said target molecule, (b) docking, using said atomic coordinates of (a), said candidate polypeptide to said target molecule to form a pseudo complex with the best intermolecular surface complementarity; (c) modeling interfacial side-chains or groups of atoms of said candidate polypeptide to generate a set of globally optimized pseudo complexes, thereby redesigning said candidate polypeptide for binding to said target molecule.

In one embodiment, said candidate polypeptide is a transcription factor, and said candidate molecule is a DNA molecule.

### BRIEF DESCRIPTION OF THE DRAWINGS

FIG. 1. A schematic drawing adapted from Gadd et al., J. Mol. Biol. 272: 106-120 (1997), which describes a general Fourier correlation docking algorithm, and which is based on the method of Katchalski-Katzir et al., P.N.A.S. USA 89: 2195-2199 (1992). Only the general steps are similar to the one described in the instant application, while the many distinct differences are apparent and described in more detail in the description of the instant application. The algorithm described in the figure uses the full atomic coordinates of molecules A and B. Molecules A and B are discretized differently. Molecule A has a negative core and a positive surface layer (the dark band) whereas no surface core distinction is made for molecule B. It is only necessary to discretize and Fourier transform molecule A one time. Electrostatic complementarity is calculated concurrently with shape complementarity. Similarly, the transform of the electric field of molecule A need only be calculated once. The cross-section of a sample 3D Fourier correlation function illustrates a search of translational space. The geometric centers of the two molecules are superposed at the origin. Molecule A is fixed in the centre of the grid. As molecule B moves through the grid, a “signal” describing shape complementarity emerges. A zero correlation score indicates that the proteins are not in contact while negative scores (the empty region in the centre) indicate significant surface penetration. The highest peak indicates the translation vector giving the best surface complementarity. FIGS. 10-12 represent actual data obtained using the instant invention.

FIG. 2. (a) The β1 domain of the Streptococcal protein G (Gβ1); (b) The initial target orientation, a dual 180° rotation about the y and z axis's of protein G, resulting in one molecule (B) flipped head-to-tail and oriented helix-face to helix-face; (c) The orientation which exhibited the highest surface complementarity between A and B (for clarity in illustrating the considerable interdigitation only the beta-sheet surface of monomer B is shown); (d) The side-chains of the 24 calculated positions. The total redesign resulted in a 20-fold mutant (12 for monomer A and 8 for B; 4 remained wild-type). Upon complex formation these mutant monomers bury ˜1560 Å2 of surface area (˜76% of which is hydrophobic).

FIG. 3. [¹⁵N, ¹H] HSQC Spectra. (A) [¹⁵N, ¹H] HSQC spectrum of uniformly enriched ¹⁵N-monomer-A alone; and (B) with equimolar quantities of unlabeled-B. The ¹⁵N-monomer-A peaks that are non-observable or exhibit chemical shift perturbations upon complex formation are labeled red. The peak labeled by * does not exhibit any NOE or TOCSY transfer in associated 3D-HSQC experiments.

FIG. 4. Chemical Shift Perturbations Mapped to the Surface of Monomer-A. The program GRASP (Nicholls et al., 1991) was used to generate the images and to map chemical shift perturbations to the surface of ¹⁵N-monomer-A. Residues that have [¹⁵N, ¹H]-HSQC peaks that are not detectable in the complex are colored dark blue and those that exhibit chemical shift changes are colored lighter blue. Monomer-B is depicted as a gray backbone worm with putative interfacial side-chains colored red. (A) interface of the target orientation and (B) surface of beta-sheet face of monomer-A (˜180° rotation of complex, monomer-B on opposite side).

FIG. 5. A) The protective antigen (PA), lethal factor (LF) and edema factor (EF) of the Anthrax toxin. B) Targeting the surface region of the protective antigen protein (PA) that becomes buried upon self-assembly into a functional heptamer (protein-G in blue and PA in gray). C) The final choice PA-Protein G complex.

FIG. 6. Fibrils of Monomer B formed in an NMR tube. The concentration of monomer B for NMR analysis was approximately 2.5 mM. The solution conditions were 25 mM phosphate buffer at pH 6.5 and 10% D₂O. Fibers were observed to spontaneously form in the NMR tube after approximately three days.

FIG. 7. Transmission electron micrograph of negatively stained image of monomer B fibrils.

FIG. 8. Thioflavine-T fluorescence emission spectra. 10 μl of single protein samples were mixed into 5 μM ThT, 0.5 M Tris-HCl, 100 mM NaCl to a final volume of 1 mL. 20 μl of complex protein samples were mixed into the same solution to account for the 0.5 fold dilution.

FIG. 9. Increase in Thioflavine-T Fluorescence: the relative fluorescence of the agitated and still protein samples in 5 μM ThT, 0.5 M Tris HCl and 100 mM NaCl. Comparison of relative fluorescence at 483 nm of Thioflavine T blank, monomer A incubated at 37° C., monomer A agitated at 37° C. and 300 rpm, wildtype Protein G incubated at 37° C., wildtype Protein G agitated at 37° C. and 300 rpm, monomer B incubated at 37° C., monomer B agitated at 37° C. and 300 rpm, and equimolar monomer A and monomer B agitated at 37° C. and 300 rpm.

FIG. 10. The Highest Scoring Docked Complex of a Protein-G/Protein-G Dimer. The Geometric Recognition Algorithm (GRA) was utilized to dock protein-G to itself. The images illustrate the high degree of surface complementarity exhibited by the top scoring complex. The knobs on one molecule fit quite well with the valleys of the other and vice versa. The top panel represents the complex with a skin drawn on the solvent accessible surface area. The bottom panel is the same image with a mesh drawn in place of the surface skin. In the bottom panel, it can be seen that the knobs and valleys are formed by the atoms left intact (i.e., the backbone atoms and the C_{β} atoms of side-chains).

FIG. 11. Representative Two-Dimensional Slice of a Three-Dimensional Correlation Map. This image is a top-down view of a 2D slice from a Geometric Recognition Algorithm (GRA) calculation in which protein-G was docked to itself The slice corresponds to the y-shift vector of the highest correlation score. The x- and z-shift vectors that correspond to the highest score are represented by a black dot and the white arrow. The relative value of the correlation score at each translational shift position is illustrated with the following coloring scheme: light blue—very negative correlation (e.g., when the molecules track through or penetrate one another), dark blue—negative correlation associated with less extensive penetration, orange—positive correlation when the amount of favorable surface complementarity out ways slight penetration, yellow corresponds to the highest regions of positive correlation and the black spot represents the shift vector with the highest docking score (i.e., the docking of highest surface complementarity, see FIG. 1). The shift vectors that correspond to a zero correlation (i.e., the molecules are not touching) are represented with the color gray. FIG. 12. Three-Dimensional Contour Map of GRA Calculation. This image is a 3D contour map of a Geometric Recognition Algorithm (GRA) calculation in which protein-G was docked to itself. It is essentially the same map as in FIG. 11 but in this case the correlation values are represented by both color and height in the third dimension. The slice corresponds to the y-shift vector of the highest correlation score. The x- and z-shift vectors that correspond to the highest score are represented by the cyan dot and the highest point. The relative value of the correlation score at each translational shift position is illustrated with the same color scheme used in FIG. 11. The structure of the highest scoring complex is shown in FIG. 10.

### DETAILED DESCRIPTION OF THE INVENTION

I. Overview

In general, the instant invention provides computational methods to design, engineer and mutate molecules, such as proteins, so that they can bind, or “dock,” to other molecules (other proteins) in a structurally specific and precise manner (i.e. as opposed to non-specific gross aggregation). Thus, in a preferred embodiment, the invention provides a method to target proteins (for example, engineered antibodies) to bind to exact regions of other proteins.

The invention provides a computational method for designing a molecule (such as a candidate protein sequence) that will be complementary to and have a binding interaction with a targeted biopolymer, such as a protein or DNA. In the first step of method, two or more proteins are computationally docked according to a general pre-defined target orientation. In one embodiment, the method implements an algorithm that treats the molecules as rigid bodies and rotates and translates their atomic coordinates within the bounds of the pre-defined orientation. Concurrently, surface shape complementarity (i.e. goodness of fit) is rigorously assessed as a function of translational and rotational position. This potentially computationally intensive process can optionally be rendered more tractable with the incorporation of the Fourier correlation theorem (FCT). The atomic coordinates which result in the highest score (i.e. exhibit the best intermolecular surface complementarity) are then used in the second part of this docking algorithm invention.

After docking of the molecules, the optimal atomic coordinates of the docked molecules are combined and treated as one single entity (complex). The combined coordinates are fed into a design algorithm, such as the ORBIT suite of design methods (U.S. Pat. No. 6,188,965 and copending U.S. patent application. Ser. No. 09/127,926, the entire contents of which are all incorporated by reference herein) which are used to computationally mutate and repack the interfacial side-chains to a more favorable energy state. If both interacting molecules are used, the interfacial side-chains are repacked in a manner analogous to the core of a well folded protein. The ORBIT algorithms score and return mutant amino acid sequences which possess the physical chemical characteristics that drive the proteins to bind together into the pre-defined target structure.

One of the most powerful advantages of the instant invention over non-computational methods is the vastly increased size of the searchable sequence space available to our overall process. The docking procedure presented herein can successfully screen a very large number (more than 1010) of possible binding geometries to a reasonable number (for example, ˜50) of predicted complexes using the native structures of the proteins. For such a small number of candidates, it is possible to use more computationally demanding techniques to refine further the few remaining complexes to account for desolvation and conformational changes.

Although the docking step of the method is related to the methods described in Katchalski-Katzir or Gabb (supra), there are important distinctions. For example, in both studies mentioned above, the methods are developed to learn how natural complexes dock together. In other words, in both studies, it is known that protein X and Y form a complex in nature, but the crystal structure of the X-Y complex is unknown, despite the fact that the crystal structures of X and Y proteins are both known. Thus, the problem is trying to predict the model structure of the X-Y complex using the compuational and physical chemical methods. Therefore, no modifications could be made, and indeed were not made, to alter either the atomic coordinates of the proteins, or to the identity of any side-chains, either prior to or after the docking of the proteins. Thus the method described above is fundamentally different from a computational algorithm aiming at redesigning novel interactions between known proteins.

II. Definitions

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. Thus such discussion should not be construed to be limiting. The scope and meaning of any use of a term will be apparent from the specific context in which the term is used.

“About” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“Amino acid” or “(amino acid) residue” includes the twenty L-amino acids commonly found in naturally occurring proteins (Ala or A, Cys or C, Asp or D, Glu or E, Phe or F, Gly or G, His or H, lie or I, Lys or K, Leu or L, Met or M, Asn or N, Pro or P, Gln or Q, Arg or R, Ser or S, Thr or T, Val or V, Trp or W, Tyr or Y, as defined and listed in WIPO Standard ST.25 (1998), Appendix 2, Table 3). “Hydrophobic residue” generally includes alanine, valine, isoleucine, leucine, phenylalanine, tyrosine, tryptophan, and methionine (in some embodiments, when the α scaling factor of the van der Waals scoring function, described below, is low, methionine is removed from the set). “Hydrophilic residue” generally includes alanine, serine, threonine aspartic acid, asparagine, glutamine, glutamic acid, arginine, lysine and histidine. Such categorization is provided for purpose of general guidance, and is thus not absolute.

“Backbone,” or “template” includes the backbone atoms of a protein (such as the N, C_{α}, carbonyl oxygen, and C in COO⁻). In certain cases, backbone may also include all fixed side-chains of the protein. When used to describe non-protein molecules, the backbone atoms include those necessary to form at least the scaffold of the molecule.

Specifically, “protein backbone structure” or grammatical equivalents herein generally refers to the three dimensional coordinates that define the three dimensional structure of a particular protein. The structures which comprise a protein backbone structure (of a naturally occurring protein) are the nitrogen, the carbonyl carbon, the α-carbon, and the carbonyl oxygen, along with the direction of the vector from the α-carbon to the β-carbon.

Depending on specific situations, the protein backbone structure which is input into the computer can either include the coordinates for both the backbone and the amino acid side-chains, or just the backbone, i.e. with the coordinates for the amino acid side-chains removed. If the former is done, the side-chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

Optionally, the protein backbone structure may be altered prior to the analysis outlined below. In this embodiment, the representation of the starting protein backbone structure is reduced to a description of the spatial arrangement of its secondary structural elements. The relative positions of the secondary structural elements are defined by a set of parameters called super-secondary structure parameters. These parameters are assigned values that can be systematically or randomly varied to alter the arrangement of the secondary structure elements to introduce explicit backbone flexibility. The atomic coordinates of the backbone are then changed to reflect the altered super-secondary structural parameters, and these new coordinates are input into the system for use in the subsequent protein design automation. For details, see U.S. Pat. No. 6,269,312, the entire content incorporated herein by reference.

“Biopolymer” includes a macromolecule that is formed by linking together two or more structurally, chemically, and/or biologically-related smaller molecules, such as a protein from amino acids, DNA from nucleotides, or polysaccharides from mono-sugar molecules. The smaller molecules need not to be identical to one another, such as the different amino acids in a protein. Biopolymer may also include molecules that are largely based on repetitive smaller structural elements, such as the CH₂ repeats in long chain fatty acids, or ring structures in steroids.

“Conformational energy” includes the energy associated with a particular “conformation”, or three-dimensional structure, of a macromolecule, such as the energy associated with the conformation of a particular protein, including two or more docket proteins treated as a single protein during the energy calculation. Interactions that tend to stabilize a protein have energies that are represented as negative energy values, whereas interactions that destabilize a protein have positive energy values. Thus, the conformational energy for any stable protein is quantitatively represented by a negative conformational energy value. Generally, the conformational energy for a particular protein will be related to that protein's stability. In particular, molecules that have a lower (i.e., more negative) conformational energy are typically more stable, e.g., at higher temperatures (i.e., they have greater “thermal stability”). Accordingly, the conformational energy of a protein may also be referred to as the “stabilization energy.”

Typically, the conformational energy is calculated using an energy “force-field” that calculates or estimates the energy contribution from various interactions which depend upon the conformation of a molecule. The force-field is comprised of terms that include the conformational energy of the alpha-carbon backbone, side-chain—backbone interactions, and side-chain—side-chain interactions. Typically, interactions with the backbone or side-chain include terms for bond rotation, bond torsion, and bond length. The backbone-side-chain and side-chain-side-chain interactions include van der Waals interactions, hydrogen-bonding, electrostatics and salvation terms. Electrostatic interactions may include Coulombic interactions, dipole interactions and quadrapole interactions). Other similar terms may also be included. Force-fields that may be used to determine the conformational energy for a polymer are well known in the art and include the CHARMM (see, Brooks et al, J. Comp. Chem. 1983,4:187-217; MacKerell et al., in The Encyclopedia of Computational Chemistry, Vol. 1:271-277, John Wiley & Sons, Chichester, 1998), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94-:8897) force-fields, to name but a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb Z S, Tidor B, “Do salt bridges stabilize proteins-a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pair-wise Calculation of Protein Solvent-Accessible Surface Areas,” Folding & Design, 3: 253-258 (1998); Eisenberg D & Wesson L, “Atomic salvation parameters applied to molecular dynamics of proteins in solution,” Protein Science, 1: 227-235 (1992); Gordon & Mayo, supra.

“Coupled residues” include residues in a molecule that interact, through any mechanism. The interaction between the two residues is therefore referred to as a “coupling interaction.” Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the “fitness” of the molecule, particularly if the change disrupts the coupling interaction between the two residues. Coupling interaction may also be described by a distance parameter between residues in a molecule. If the residues are within a certain cutoff distance, they are considered interacting.

“Dock” can be used to describe one molecule (protein) binding to one or more other molecules (proteins) in a structurally specific and precise manner (i.e. as opposed to non-specific gross aggregation). Preferably, the binding surfaces of the binding partners fit seamlessly or nearly seamlessly together, such that interacting residues belonging to two binding partners interact in such as way as if they were internal residues of a single macromolecule (such as a single protein). In a preferred embodiment, one protein (for example, an engineered antibody) is specifically targeted to bind to an exact region(s) of one or more other proteins.

“Docking surface” includes, minimally, a surface of a molecule (candidate polypeptide of target biopolymer) used for docking. The detail of the surface is largely dependent on the level of molecular details provided by the atomic coordinates (or atomic coordinate model) of the molecule. Certain details, such as the presence or absence of the H atoms, amino acid side-chains or portions thereof, the associated charges, etc., may be omitted in certain models (“stripped” or “shaved” models) based on predefined criteria. For the purpose of certain calculation algorithms, the surface can be treated as a rigid surface. Alternatively, the surface may be softened by allowing a predefined “surface thickness” to partly compensate for certain stripped models, including models with stripped H atoms.

“Atomic coordinate model” usually derives from three-dimensional structure coordinates of molecules of interest, or homologs thereof with similar structure. However, certain atomic coordinate models may omit certain levels of details provided by the original, complete atomic coordinates. For example, the model may not have any terminal H atoms; or may only include backbone atoms of a protein; or may include no more than C_{β} atoms of amino acid side-chain atoms, either for the surface/solvent-exposed residues or for the whole protein; etc.

“Fitness” may be used to denote the level or degree to which a particular property or a particular combination of properties for a molecule, e.g., a protein, are optimized. In certain embodiments of the invention, the fitness of a protein is preferably determined by properties which a user wishes to improve. Thus, for example, the fitness of a protein may refer to the protein's thermal stability, catalytic activity, binding affinity, solubility (e.g., in aqueous or organic solvent), and the like. Other examples of fitness properties include enantioselectivity, activity towards non-natural substrates, and alternative catalytic mechanisms. Coupling interactions can be modeled as a way of evaluating or predicting fitness (stability). Fitness can be determined or evaluated experimentally or theoretically, e.g. computationally.

Preferably, the fitness is quantitated so that each molecule, e.g., each amino acid will have a particular “fitness value”. For example, the fitness of a protein may be the rate at which the protein catalyzes a particular chemical reaction, or the protein's binding affinity for a ligand. In a particularly preferred embodiment, the fitness of a protein refers to the conformational energy of the polymer and is calculated, e.g., using any method known in the art. See, e.g. Brooks B. R., Bruccoleri R E, Olafson, B D, States D J, Swaminathan S & Karplus M, “CHARMM: A Program for Macromolecular Energy, Minimization, and Dynamics Calculations,” J. Comp. Chem., 4: 187-217 (1983); Mayo S L, Olafson B D & Goddard W A G, “DREIDING: A Generic Force Field for Molecular Simulations,” J. Phys. Chem., 94: 8897-8909 (1990); Pabo C 0 & Suchanek E G, “Computer-Aided Model-Building Strategies for Protein Design,” Biochemistry, 25: 5987-5991 (1986), Lazar G A, Desjarlais J R & Handel T M, “De Novo Design of the Hydrophobic Core of Ubiquitin,” Protein Science, 6: 1167-1178 (1997); Lee C & Levitt M, “Accurate Prediction of the Stability and Activity Effects of Site Directed Mutagenesis on a Protein Core,” Nature, 352: 448-451 (1991); Colombo G & Merz K M, “Stability and Activity of Mesophilic Subtilisin E and Its Thermophilic Homolog: Insights from Molecular Dynamics Simulations,” J. Am. Chem. Soc., 121: 6895-6903 (1999); Weiner S J, Kollman P A, Case D A, Singh U C, Ghio C, Alagona G, Profeta S J, Weiner P, “A new force field for molecular mechanical simulation of nucleic acids and proteins,” J. Am. Chem. Soc., 106: 765-784 (1984). Generally, the fitness of a protein is quantitated so that the fitness value increases as the property or combination of properties is optimized. For example, in embodiments where the thermal stability of a protein is to be optimized (conformational energy is preferably decreased), the fitness value may be the negative conformational energy; i.e., F=−E.

The “fitness contribution” of a protein residue may refer to the level or extent f(iₐ) to which the residue iₐ, having an identity a, contributes to the total fitness of the protein. Thus, for example, if changing or mutating a particular amino acid residue will greatly decrease the protein's fitness, that residue is said to have a high fitness contribution to the polymer. By contrast, typically some residues iₐ in a protein may have a variety of possible identities a without affecting the protein's fitness. Such residues, therefore have a low contribution to the protein fitness.

“Dead-end elimination” (DEE) is a deterministic search algorithm that seeks to systematically eliminate bad rotamers and combinations of rotamers until a single solution remains. For example, amino acid residues can be modeled as rotamers that interact with a fixed backbone. The theoretical basis for DEE provides that, if the DEE search converges, the solution is the global minimum energy conformation (GMEC) with no uncertainty (Desmet et al., 1992).

Dead end elimination is based on the following concept. Consider two rotamers, iᵣ and iₜ, at residue i, and the set of all other rotamer configurations {S} at all residues excluding i (of which rotamer jₛ is a member). If the pair-wise energy contributed between iᵣ and jₛ is higher than the pair-wise energy between iₜ and jₛ for all {S}, then rotamer iᵣ cannot exist in the global minimum energy conformation, and can be eliminated. This notion is expressed mathematically by the inequality.

$$E ( {i}_{r} ) + {\sum }_{j \neq i}^{N} E ( {i}_{r} , {j}_{s} ) > E ( {i}_{t} ) + {\sum }_{j \neq i}^{N} E ( {i}_{t} , {j}_{s} ) \{ S \} \qquad ( Equation A )$$

If this expression is true, the single rotamer iᵣ can be eliminated (Desmet et al., 1992).

In this form, Equation A is not computationally tractable because, to make an elimination, it is required that the entire sequence (rotamer) space be enumerated. To simplify the problem, bounds implied by Equation A can be utilized:

$$E ( {i}_{r} ) + {\sum }_{j \neq i}^{N} min ( s ) E ( {i}_{r} , {j}_{s} ) > E ( {i}_{t} ) + {\sum }_{j \neq i}^{N} max ( s ) E ( {i}_{t} , {j}_{s} ) \{ S \} \qquad ( Equation B )$$

Using an analogous argument, Equation B can be extended to the elimination of pairs of rotamers inconsistent with the GMEC. This is done by determining that a pair of rotamers ir at residue i and jₛ at residue j, always contribute higher energies than rotamers iₐ and jᵥ with all possible rotamer combinations {L}. Similar to Equation B, the strict bound of this statement is given by:

$$ϵ ( {i}_{r} , {j}_{s} ) + {\sum }_{k \neq i , j}^{N} min ( t ) ϵ ( {i}_{r} , {j}_{s} , {k}_{t} ) > ϵ ( {i}_{u} , {j}_{v} ) + {\sum }_{k \neq i , j}^{N} max ( t ) ϵ ( {i}_{u} , {j}_{v} , {k}_{i} ) \qquad ( Equation C )$$

where ε is the combined energies for rotamer pairs

ε(iᵣ,jₛ)=E(iᵣ)+E(jₛ)+E(iᵣ,jₛ) (Equation D), and

ε(iᵣ,jₛ,kₜ)=E(iᵣ,kₜ)+E(jₛ,kₜ) (Equation E).

This leads to the doubles elimination of the pair of rotamers ir and js, but does not eliminate the individual rotamers completely as either could exist independently in the GMEC. The doubles elimination step reduces the number of possible pairs (reduces S) that need to be evaluated in the right-hand side of Equation 6, allowing more rotamers to be individually eliminated.

The singles and doubles criteria presented by Desmet et al. fail to discover special conditions that lead to the determination of more dead-ending rotamers For instance, it is possible that the energy contribution of rotamer it is always lower than ir without the maximum of it being below the minimum of iᵣ. To address this problem, Goldstein 1994 presented a modification of the criteria that determines if the energy profiles of two rotamers cross. If they do not, the higher energy rotamer can be determined to be dead-ending. The doubles calculation significantly more computational time than the singles calculation. To accelerate the process, other computational methods have been developed to predict the doubles calculations that will be the most productive (Gordon & Mayo, 1998). These kinds of modifications, collectively referred to as fast doubles, significantly improved the speed and effectiveness of DEE.

Several other modifications also enhance DEE. Rotamers from multiple residues can be combined into so-called super-rotamers to prompt further eliminations (Desmet et al., 1994; Goldstein, 1994). This has the advantage of eliminating multiple rotamers in a single step. In addition, it has been shown that “splitting” the conformational space between rotamers improves the efficiency of DEE (Pierce et al., 2000). Splitting handles the following special case. Consider rotamer iᵣ. If a rotamer iₜ₁ contributes a lower energy than iᵣ for a portion of the conformational space, and a rotamer iₜ₂ has a lower energy than iᵣ for the remaining fraction, then iᵣ can be eliminated. This case would not be detected by the less sensitive Desmet or Goldstein criteria. In the preferred implementations of the invention as described herein, all of the described enhancements to DEE were used.

For further discussion of these methods see, Goldstein, R. F. (1994), Efficient rotamer elimination applied to protein side-chains and related spin glasses, Biophysical Journal 66, 1335-1340; Desmet, J., De Maeyer, M., Hazes, B. & Lasters, I. (1992), The dead-end elimination theorem and its use in protein side-chain positioning. Nature 356,539-542; Desmet, J., De Maeyer, M. & Lasters, I. (1994), In The Protein Folding Problem and Tertiary Structure Prediction (Jr., K. M. & Grand, S. L., eds.), pp. 307-337 (Birkhauser, Boston); De Maeyer, M., Desmet, J. & Lasters, I. (1997), All in one: a highly detailed rotamer library improves both accuracy and speed in the modeling of side-chains by dead-end elimination, Folding & Design 2, 53-66, Gordon, D. B. & Mayo, S. L. (1998), Radical performance enhancements for combinatorial optimization algorithms based on the dead-end elimination theorem, Journal of Computational Chemistry 19, 1505-1514; Pierce, N. A., Spriet, J. A., Desmet, J., Mayo, S. L., (2000), Conformational splitting: A more powerful criterion for dead-end elimination; Journal of Computational Chemistry 21, 999-1009.

“Expression system” includes a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

“Favorable interaction” and the related “non-favorable interaction” may refer to, energy wise, whether a specific residue is still favored to be present at a given interfacial position upon complex formation, since these interfacial residues used to be surface-exposed residues before complex formation. Thus energy wise, a former surface residue in one of the interacting proteins may form more favorable interactions with the same target when mutated as a core residue.

“Host cell” includes any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells may be cultured in vitro or one or more cells in a non-human animal (e.g., a transgenic animal or a transiently transfected animal).

The methods of the invention may include steps of comparing sequences to each other, including wild-type sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “−”, or “Δ”) in the polymer sequence not containing the inserted or deleted residue.

“Homologous”, in all its grammatical forms and spelling variations, generally refers to the relationship between two proteins that possess a “common evolutionary origin”, including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, can be used to describe the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., Molecular Cloning: A Laboratory Manual, Second Edition (1989) Cold Spring Harbor Laboratory Press, Cold Spring Harbor, N.Y.). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is a 0.15M NaCl, 0.01 μM Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

“Interface” or “binding interface” may include the collection of atoms occupying the surface area of the molecules in direct contact with its binding partner. Interface may additional include atoms that are sufficiently close (for example, less than 15 Å, 10 Å, 8 Å, 5 Å, 2 Å, 1 Å, or less) to atoms of the binding partner.

Amino acid residues on a candidate (or on a target) polypeptide that are in direct contact with one or more amino acids on a target (or a candidate) polypeptide are called (direct-contact) “interfacial residues.” Interfacial residues may also include those amino acid residues on the candidate or the target polypeptide which are in close proximity to those direct-contact interfacial residues (proximity interfacial residues). “Close proximity” means either direct contact through covalent bonding (such as peptide bond or disulfide bond) or within 5 Å, preferably 3 Å, 2 Å, 1 Å or less. Alternatively, any residues with any of its atoms within a given distance (for example, 15 Å, 12 Å, 10 Å, 8 Å, 5 Å or less) of the binding partner comprises the interface residues.

“Polypeptide,” “peptide” or “protein” are used interchangeably to describe a chain of amino acids that are linked together by chemical bonds called “peptide bonds.” A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

“Rotamer” is a set of possible conformers for each amino acid or analog side-chain. See Ponder, et al., Acad. Press Inc. (London) Ltd. pp. 775-791 (1987); Dunbrack, et al, Struc. Biol. 1(5):334-340 (1994); Desmet, et al., Nature 356:539-542 (1992). A “rotamer library” is a collection of a set of possible/allowable rotametic conformations for a given set of amino acids or analogs. There are two general types of rotamer libraries: “backbone dependent” and “backbone independent.” A backbone dependent rotamer library allows different rotamers depending on the position of the residue in the backbone; thus for example, certain leucine rotamers are allowed if the position is within an α helix, and different leucine rotamers are allowed if the position is not in an α-helix. A backbone independent rotamer library utilizes all rotamers of an amino acid at every position. In general, a backbone independent library is preferred in the consideration of core residues, since flexibility in the core is important. However, backbone independent libraries are computationally more expensive, and thus for surface and boundary positions, a backbone dependent library is preferred. However, either type of library can be used at any position.

“Variable residue position” includes an amino acid position of the protein to be designed that is not fixed in the design method as a specific residue or rotamer, generally the wild-type residue or rotamer. It should be noted that even if a position is chosen as a variable position, it is possible that the methods of the invention will optimize the sequence in such a way as to select the wild type residue at the variable position. This generally occurs more frequently for core residues, and less regularly for surface residues. In addition, it is possible to fix residues as non-wild type amino acids as well.

“Fixed residue position” includes residues identified in the three dimensional structure as being in a set conformation. In some embodiments, a fixed position is left in its original conformation (which may or may not correlate to a specific rotamer of the rotamer library being used). Alternatively, residues may be fixed as a non-wild type residue depending on design needs; for example, when known site-directed mutagenesis techniques have shown that a particular residue is desirable (for example, to eliminate a proteolytic site or alter the substrate specificity of an enzyme), the residue may be fixed as a particular amino acid. Residues which can be fixed include, but are not limited to, structurally or biologically functional residues.

In certain embodiments, a fixed position may be “floated”; the amino acid or analog at that position is fixed, but different rotamers of that amino acid or analog are tested. In this embodiment, the variable residues may be at least one, or anywhere from 0.1% to 99.9% of the total number of residues. Thus, for example, it may be possible to change only a few (or one) residues, or most of the residues, with all possibilities in between.

“Surface shape complementarity,” “goodness of (surface) fit” or “surface-to-surface geometric fitting” generally refers to the degree of geometric surface match or complementation between two or more potentially interacting molecules. The potentially interacting molecules have a better degree of surface shape complementarity if the gap in the interface between the interacting molecules are smaller, such that the molecules tightly hug one another based on the shape of the surface contour. Surface shape complementarity in the geometric sense does not, however, include considerations such as electrostatic forces or other biochemical information. Surface shape complementarity can be evaluated/calculated as a function of translational and rotational positions of the involved molecules, using the quantitative methods described in the instant application (usually treating the shapes as rigid bodies). In certain embodiment, the calculations can be carried out alone. In other embodiment, the calculations can be combined with additional calculations that consider one or more non-geometnc factors mentioned above. In all these calculations, a score is obtained as the result of the calculation. That score provides a quantitative measure for degrees of surface shape complementarity. Thus “bad” surface shape complementarity with a score lower than a preset value can be discarded without further consideration. The process of identifying the best surface shape complementation between potential binding partners (optionally including considering one or more non-geometric factors) is called “docking” (or all its grammatical variations).

“Rotation” or all its grammatical variations as used in “rotational movement” can be used to describe motion of a body/object characterized by turning around on one or more axises or center. For example, the pure rotational movement a free object can be defined by its rotation around three axises in the three demension. In other words, the three dimensional orientation of a free object can be defined by three Euler angles (see Goldstein, H., in Classical Mechanics, by Addison-Wesley, Reading, Mass., p. 608, 1980, incorporated herein by reference).

“Translation” or all its grammatical variations as used in “translational movement” can be used to describe motion of a body or an object in which every point of the body/object moves parallel to and the same distance as every other point of the body/object. Alternatively, it means motion in which all the points of the moving body have at any instant the same velocity and direction of motion (as opposed to rotational movement).

“Optimal” as used in “optimal atomic coordinates associated with the best intermolecular surface complementarity” may include a list of the best possible intermolecular surface complimentarity,” all of which has met a pre-determined cut-off value (for example, the best 4,000 possible surface complimentarity in a given calculation with a given parameters). In a reiterative search mode, where multiple rounds of calculations are done using different parameters, the list of optimal complexes with the best surface complimentarity may vary from round to round, both in relative rank of the goodness of fit and in the number of all listed complexes. Typically, a global search is generally done in the initial stage (called the “scan, stage”) with coarse parameters. The search can be refined during subsequent rounds (called the “discrimination stage”) with more fine-tuned parameters.

III. Illustrative Embodiments

A. Atomic Coordinates and Other Sequence/Structural Information for Proteins

An accurate description of the candidate and target molecules (such as candidate and target proteins) using the terms of atomic coordinates is important for the computational design approach of the instant invention. Although the crystal structure of a protein will provide the exact backbone and the C_{β} atoms coordinates, in many cases, it is perfectly acceptable to use crystal structure of a homologous protein (for example, a homolog from a related species) or even a conserved domain to substitute the crystallographic structure description for the backbone and the C_{β} atoms.

The crystal structures of thousands of proteins are currently available in the Brookhaven Protein Data Bank (PDB, see Bernstein et al., J. Mol. Biol. 112: 535-542, 1977). All contents of PDB are in the public domain. As of Mar. 25, 2003, PDB contains 20,473 total deposited structures, including 18,434 protein/peptide/virus structures, 854 protein/nucleic acid complex structures, 1167 nucleic acid structures, and 18 carbohydrates. Presently, about 4000-4500 structures are being deposited to this database every year. More detailed information regarding all aspects of the PDB database can be found at the PDB website.

The structure database or Molecular Modeling DataBase (MMDB) contains experimental data from crystallographic and NMR structure determinations. The data for MMDB are obtained from the Protein Data Bank (PDB). The NCBI (National Center for Biotechnology Information) has cross-linked structural data to bibliographic information, to the sequence databases, and to the NCBI taxonomy. Cn3D, the NCBI 3D structure viewer, can be used for easy interactive visualization of molecular structures from Entrez.

The Entrez 3D Domains database contains protein domains from the NCBI Conserved Domain Database (CDD). Computational biologists define conserved domains based on recurring sequence patterns or motifs. CDD currently contains domains derived from two popular collections, Smart and Pfam, plus contributions from colleagues at NCBI, such as COG. The source databases also provide descriptions and links to citations. Since conserved domains correspond to compact structural units, CDs contain links to 3D-structure via Cn3D whenever possible.

To identify conserved domains in a protein sequence, the CD-Search service employs the reverse position-specific BLAST algorithm. The query sequence is compared to a position-specific score matrix prepared from the underlying conserved domain alignment. Hits may be displayed as a pair-wise alignment of the query sequence with a representative domain sequence, or as a multiple alignment. CD-Search now is run by default in parallel with protein BLAST searches. While the user waits for the BLAST queue to further process the request, the domain architecture of the query may already be studied. In addition, CDART, the Conserved Domain Architecture Retrieval Tool allows user to search for proteins with similar domain architectures. CDART uses precomputed CD-search results to quickly identify proteins with a set of domains similar to that of the query. For more details, see Marchler-Bauer et al., Nucleic Acids Research 31: 383-387, 2003; and Marchler-Bauer et al., Nucleic Acids Research 30: 281-283, 2002.

All these databases would provide atomic coordinates for proteins or other molecules with known structural information.

Alternatively, in certain embodiments, if the exact crystal structure of a particular protein/molecule is unknown, but its protein sequence is similar or homologous to a known protein sequence with a known crystal structure. In such instances, it is expected that the conformation of the protein in question will be similar to the known crystal structure of the homologous protein. The known structure may, therefore, be used as the structure for the protein of interest, or more preferably, may be used to predict the structure of the protein of interest (i.e., in “homology modeling” or “molecular modeling”). As a particular example, the Molecular Modeling Database (MMDB) described above (see, Wang et al., Nucl. Acids Res. 2000, 28:243-245; Marchler-Bauer et al., Nucl. Acids Res. 1999,27:240-243) provides search engines that may be used to identify proteins and/or nucleic acids that are similar or homologous to a protein sequence (referred to as “neighboring” sequences in the MMDB), including neighboring sequences whose three-dimensional structures are known. The database further provides links to the known structures along with alignment and visualization tools, such as Cn3D (developed by NCBI), RasMol, etc., whereby the homologous and parent sequences may be compared and a structure may be obtained for the parent sequence based on such sequence alignments and known structures.

The homologous protein sequence with known 3D-structure is preferably at least about 60%, or at least about 70%, or at least about 80%, or at least about 90%, or at least about 95% identical to the protein of interest, at least in the region that may be involved in interacting with another molecule of interest. Such potential binding sites may not be continuous in the primary amino acid sequence of the protein since distant amino acids may come together in the 3D-structure. In this case, sequence homology or identity can be calculated using, for example, the NCBI standard BLASTp programs for protein using default conditions, in regions aligned together (without insertions or deletions in either of the two sequences being compared) and including residues known to be involved in substrate amino acid binding. Alternatively, the homologous protein is preferably about 35%, or 40%, or 45%, or 50%, or 55% identical overall to the protein of interest. Many proteins with just about 20-25% overall sequence homology/identity turns out to be conserved in three-dimensional structure.

In the few cases where the structure for a particular protein sequence may not be known or available, it is typically possible to determine the structure using routine experimental techniques (for example, X-ray crystallography and Nuclear Magnetic Resonance (NMR) spectroscopy) and without undue experimentation. See, e.g., NMR of Macromolecules: A Practical Approach, G. C. K. Roberts, Ed., Oxford University Press Inc., New York (1993); Ishima R, Torchia D A, “Protein Dynamics from NMR,” Nat Struct Biol, 7: 740-743 (2000); Gardner K H, Kay L E, “The use of H-2, C-13, N-15 multidimensional NMR to study the structure and dynamics of proteins,” Annu. Rev. Bioph. Biom., 27: 357-406 (1998); Kay LE, “NMR methods for the study of protein structure and dynamics,” Biochem Cell Biol, 75: 1-15 (1997); Dayie K T, Wagner G, Lefevre J F, “Theory and practice of nuclear spin relaxation in proteins,” Annu Rev Phys Chem, 47: 243-282 (1996); Wuthrich K, “NMR—This and other methods for protein and nucleic-acid structure determination,” Acta Cyrstallogr. D, 51: 249-270 (1995); Kahn R, Carpentier P, Berthet-Colominas C, et al., “Feasibility and review of anomalous X-ray diffraction at long wavelengths in materials research and protein crystallography,” J. Synchrotron Radiat., 7: 131-138 (2000); Oakley A J, Wilce M C J, “Macromolecular crystallography as a tool for investigating drug, enzyme and receptor interactions,” Clin. Exp. Pharmacol. P., 27:145-151 (2000); Fourme R, Shepard W, Schiltz M, et al., “Better structures from better data through better methods: a review of developments in de novo macromolecular phasing techniques and associated instrumentation at LURE,” J. Synchrotron Radiat., 6: 834-844 (1999).

Alternatively, and in less preferable embodiments, the three-dimensional structure of a protein sequence may be calculated from the sequence itself and using ab initio molecular modeling techniques already known in the art. See e.g., Smith T F, LoConte L, Bienkowska J, et al., “Current limitations to protein threading approaches,” J. Comput. Biol., 4: 217-225 (1997); Eisenhaber F, Frommel C, Argos P, “Prediction of secondary structural content of proteins from their amino acid composition alone 2. The paradox with secondary structural class,” Proteins, 24: 169-179 (1996); Bohm G, “New approaches in molecular structure prediction,” Biophys Chem., 59: 1-32 (1996); Fetrow J S, Bryant S H, “New programs for protein tertiary structure prediction,” BioTechnol., 11: 479-484, (1993); Swindells M B, Thorton J M, “Structure prediction and modeling,” Curr. Opin. Biotech., 2: 512-519 (1991); Levitt M, Gerstein M, Huang E, et al., “Protein folding: The endgame,” Annu. Rev. Biochem., 66: 549-579 (1997). Eisenhaber F., Persson B., Argos P., “Protein-structure prediction—recognition of primary, secondary, and tertiary structural features from amino-acid-sequence,” Crit Rev Biochem Mol, 30:1-94(1995); Xia Y, Huang E S, Levitt M, et al., “Ab initio construction of protein tertiary structures using a hierarchical approach,” J. Mol. Biol., 300:171-185 (2000); Jones D T, “Protein structure prediction in the post genomicera,” Curr Opin Struc Biol, 10: 371-379 (2000). Three-dimensional structures obtained from ab initio modeling are typically less reliable than structures obtained using empirical (e.g., NMR spectroscopy or X-ray crystallography) or semi-empirical (e.g., homology modeling) techniques. However, such structures will generally be of sufficient quality, although less preferred, for use in the methods of this invention.

Although the above discussion uses protein as an illustrative example, other molecules (such as small chemical compounds with less than 5000 kDa) may also be similarly modeled using art-recognized molecular modeling techniques. For additional details of predicting 3D structure, see section B below.

B. Methods for Predicting 3D Structure based on Sequence Homology

For proteins that have not been crystallized or been the focus of other structural determinations, a computer-generated molecular model of the protein and its potential binding site(s) can nevertheless be generated using any of a number of techniques available in the art. For example, the Ca-carbon positions of a protein sequence of interest can be mapped to a particular coordinate pattern of a protein (“known protein”) having a similar sequence and deduced structure using homology modeling techniques, and the structure of the protein of interest and velocities of each atom calculated at a simulation temperature (To) at which a docking simulation with an amino acid analog is to be determined. Typically, such a protocol involves primarily the prediction of side-chain conformations in the modeled protein of interest, while assuming a main-chain trace taken from a tertiary structure, such as provided by the known protein. Computer programs for performing energy minimization routines are commonly used to generate molecular models. For example, both the CHARMM (Brooks et al. (1983) J Comput Chem 4:187-217) and AMBER (Weiner et al (1981) J. Comput. Chem. 106: 765) algorithms handle all of the molecular system setup, force field calculation, and analysis (see also, Eisenfield et al. (1991) Am JPhysiol 261:C376-386; Lybrand (1991) J Pharm Belg 46-49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488). At the heart of these programs is a set of subroutines that, given the position of every atom in the model, calculate the total potential energy of the system and the force on each atom. These programs may utilize a starting set of atomic coordinates, the parameters for the various terms of the potential energy function, and a description of the molecular topology (the covalent structure). Common features of such molecular modeling methods include: provisions for handling hydrogen bonds and other constraint forces; the use of periodic boundary conditions; and provisions for occasionally adjusting positions, velocities, or other parameters in order to maintain or change temperature, pressure, volume, forces of constraint, or other externally controlled conditions.

Most conventional energy minimization methods use the input coordinate data and the fact that the potential energy function is an explicit, differentiable function of Cartesian coordinates, to calculate the potential energy and its gradient (which gives the force on each atom) for any set of atomic positions. This information can be used to generate a new set of coordinates in an effort to reduce the total potential energy and, by repeating this process over and over, to optimize the molecular structure under a given set of external conditions. These energy minimization methods are routinely applied to molecules similar to the subject proteins.

In general, energy minimization methods can be carried out for a given temperature, Ti, which may be different than the docking simulation temperature, To. Upon energy minimization of the molecule at Ti, coordinates and velocities of all the atoms in the system are computed. Additionally, the normal modes of the system are calculated. It will be appreciated by those skilled in the art that each normal mode is a collective, periodic motion, with all parts of the system moving in phase with each other, and that the motion of the molecule is the superposition of all normal modes. For a given temperature, the mean square amplitude of motion in a particular mode is inversely proportional to the effective force constant for that mode, so that the motion of the molecule will often be dominated by the low frequency vibrations.

After the molecular model has been energy minimized at Ti, the system is “heated” or “cooled” to the simulation temperature, To, by carrying out an equilibration run where the velocities of the atoms are scaled in a step-wise manner until the desired temperature, To, is reached. The system is further equilibrated for a specified period of time until certain properties of the system, such as average kinetic energy, remain constant. The coordinates and velocities of each atom are then obtained from the equilibrated system.

Further energy minimization routines can also be carried out. For example, a second class of methods involves calculating approximate solutions to the constrained EOM for the protein. These methods use an iterative approach to solve for the Lagrange multipliers and, typically, only need a few iterations if the corrections required are small. The most popular method of this type, SHAKE (Ryckaert et al. (1977) J Comput Phys 23:327; and Van Gunsteren et al. (1977) Mol. Phys 34:1311) is easy to implement and scales as O(N) as the number of constraints increases. Therefore, the method is applicable to macromolecules. An alternative method, RATTLE (Anderson (1983) J Comput Phys 52:24) is based on the velocity version of the Verlet algorithm. Like SHAKE, RATTLE is an iterative algorithm and can be used to energy minimize the model of a subject protein.

After obtaining the atomic coordinates of the candidate and the target proteins, a two-step approach described below using such atomic coordinates can be employed to identify and then optimize binding sites.

C. Computational Docking and Maximization of Surface Complementarity (Step 1)

Once the general orientation of the target molecule/protein is dictated (fixed), it is essential to rigorously search local interfacial space to find the optimal surface-to-surface geometric fit between the proteins. Protein surfaces are not homogeneous and a proper fit between docked proteins needs to be assessed systematically. The same is true for other macromolecule interactions. The docking algorithms are therefore designed to rotate and translate the atomic coordinates of the molecules while rigorously searching interfacial space and scoring the various intermolecular orientations as a function of surface complementarity. In other words, the docking step includes a global search of translational and rotational space, and optionally followed by refinement of the best predictions. To accomplish this, a geometric recognition algorithm (Katchalski-Katzir et al, 1992, supra; Gabb et al 1997, supra, entire contents all incorporated herein by reference) currently used in the field of native protein docking were altered and customized as following.

Geometric Recognition Algorithm

Briefly, the geometric recognition algorithm (GRA) treats the two potentially interacting molecules as rigid bodies and uses surface complementarity as the criteria for goodness of fit.

The method begins with a geometric description of the two molecules (such as the candidate and the target polypeptides) derived from their known atomic coordinates (see above). These two molecules, denoted a (target molecule) and b (candidate molecule), are computationally projected onto a three-dimensional grid of N×N×N points. Each grid point is a “node” of the three-dimensional grid. Thus the total number of nodes in a grid of N×N×N points is N³. One of the unique steps of this process entails stripping off all the coordinates of the side-chain atoms of molecule b except those of the C_{β} atoms. Although in certain embodiments, all side-chain atoms are stripped, leaving only atomic coordinates for the backbone. In other embodiments, only the surface (exposed or water accessible) residues are stripped off their side-chain atom coordinates. For molecule a, it is preferred that the whole coordinates are used, although the side-chain coordinates may be stripped to different degrees as in molecule b.

For Gly, which does not have C_{β} atom, no stripping is necessary. For Pro, either no stripping is performed, or stripping to C_{α} or C_{β} can be done as in other residues.

The coordinates of the backbone and C_{β} atoms projected onto the three-dimensional grid of N×N×N points are then represented by the following discrete functions:

a_{l,m,n}=(I)1, if on the surface of the molecule a; (II) ρ, if inside the molecule a; or (III) 0, if outside the molecule a. [Eq. 1a]

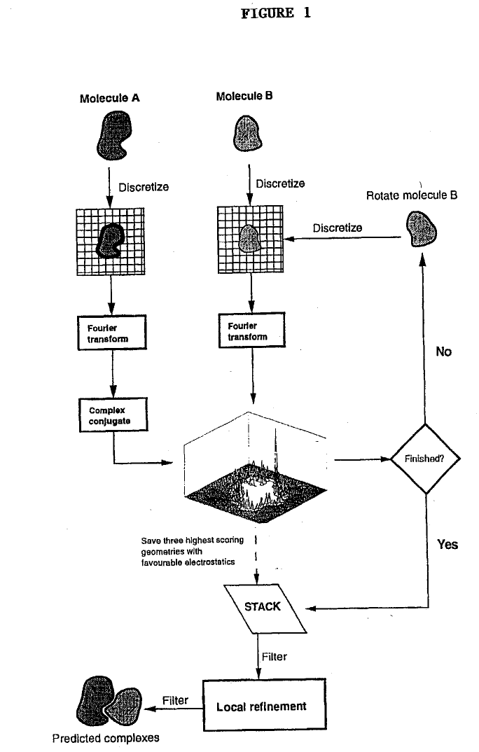

b_{l,m,n}=(I)δ, if inside the molecule b; or (II) 0, if outside the molecule b. [Eq. 1b]

where l, m, and n are the indices of the 3D grid (l, m, n={1 . . . N}). Any grid point is considered inside the molecule if there is at least one atom nucleus within a distance r from it, where r is of the order of van der Waals atomic radii. Examples for two-dimensional cross sections of these functions are presented in FIG. 1a and b in Katchalski-Katzir et al, 1992, supra.

The surface is defined here as a boundary layer of finite width between the inside and the outside of the molecule. The parameters ρ and δ describe the value of the points inside the molecules, and all points outside are set to zero.

Matching of surface complementary is accomplished by computing the following correlation function (Katchalski-Katzir et al, 1992, supra; Gabb et al. 1997, supra, entire contents incorporated herein by reference).

$$\text{Correlation Function} {c}_{α , β , γ} = {\sum }_{l = 1}^{N} {\sum }_{m = 1}^{N} {\sum }_{n = 1}^{N} {a}_{l , m , n} \times {b}_{l + α , m + β , n + γ} \qquad [ Eq . 2 ]$$

where α, β, and γ are the number of grid steps by which molecule b is shifted with respect to molecule a in each dimension.

In general, the correlation function works as follows: the position of molecule a is held constant while molecule b is shifted through three degrees of translational freedom, preferably starting by superimposing the centers of molecules a and b. The subsequence translational movements of molecule b are represented by the shift vector of values α, β and γ (i.e. the number of grid steps in each dimension). If the shift vector is such that there is no contact between the molecules the correlation value is zero. If there is good contact between the surfaces the contribution to the correlation value is positive. Finally, since molecular penetration is physically forbidden, a distinction between surface contact and penetration is made. A penalty for penetration is achieved by assigning a negative value to the inside of molecule α. Thus, shift vectors which result in significant penetration will return a large negative correlation value while positive correlation values are obtained when the contributions from surface contact outweighs those from penetration (Katchalski-Katzir et al, 1992; Gabb et al 1997). Upon completion of the translational grid search molecule b is rotated and the entire process is run again for each degree of rotational freedom.

To illustrate, if the shift vector {α, β, γ} is such that there is no contact between the two molecules, the correlation value is zero. If there is a contact between the surfaces, the contribution to the correlation value is positive. Non-zero correlation values could also be obtained when one molecule penetrates into the other. Since such penetration is physically forbidden, a distinction between surface contact and penetration must be clearly formulated. To do so, we assign large negative values to ρ in a_{l,m,n} and small non-negative values to δ in b_{l,m,n}. Thus, when the shift vector {α, β, γ} is such that molecule b penetrates molecule a, the multiplication of the negative numbers (ρ) in a_{l,m,n} by the positive numbers (1 or δ) in b_{l,m,n} results in a negative contribution to the overall correlation value. Consequently, the correlation value for each displacement is simply the score for overlapping surfaces corrected by the penalty for penetration.

Positive correlation values are obtained when the contribution from surface contact outweighs that from penetration. Thus, a good geometric match is represented by a high positive peak, and low values reflect a poor match between the molecules. A cross section of a typical correlation function for a good match is similar to what is presented in FIG. 1. The coordinates of the prominent peak denote the relative shift of molecule b yielding a good match with molecule a. The location of the recognition sites on the surface of each molecule can readily be determined from these coordinates. In addition, the width of the peak provides a measure for the relative displacement allowed before matching is lost.

In certain high resolution calculations (i.e. small grid steps) of the above correlation function, the calculations can be computationally intensive since they involve N³ multiplications and additions for each of the six degrees of translational and rotational freedom. In fact a complete calculation of interfacial space entails approximately 2×N⁹ total calculations (N³ multiplications and additions×N³ translational×˜N³ angular degrees of freedom). Although this approach is distinctly different from other methods (i.e. the relative orientation is dictated and therefore not all degrees of positional freedom need to be searched) the calculation of the correlation function remains intensive due to the desire to perform as high a resolution grid search as possible (i.e. large values for IV). To maintain high resolution while reducing the computational complexity, the Fourier correlation algorithm is incorporated (with modifications appropriate to fit this unique approach) into the docking algorithm (see below).

Although the above three-dimensional grid is cube-shaped with equal number of nodes at all three dimensions, in certain embodiments of the invention, the number of nodes at the three axises can be different from one another (for example, the 3D grid can be a 100×150×300 grid, depending on the overall three-dimensional shapes of the molecules of interest).

In addition, in certain embodiments, the overall size of the three-dimensional grid may encompass all atoms of the target protein and all atoms of the candidate protein. For example, the size of the grid may be the sum of the radii of said candidate polypeptide and said target biopolymer plus 0.5, 1, 2, or 5 Å.

Alternatively, in a related embodiment, the grid may only be focused onto a specific region of the target protein, while encompassing all the candidate molecules, or the part of the candidate molecule docking with the target protein. For example, when targeting the PA protein of the Anthrax toxin or the Tyrosyl Phosphodiesterase, the grid was focused onto a specific region of the target protein in both cases. The PA protein might have an overall dimension of about 75 Å×50 Å×50 521 if not greater. However, a grid that has an N (number of nodes) of either 128 or 64 may be used initially, but it has been shrunk down to as little as 42×42×42. This leaves much of the target molecule (e.g., PA) hanging out of the grid. Meanwhile, the “candidate” molecule (e.g., protein-G in the example below) is always well within the confines of the grid in that example. This enables significant reduction of the time length needed for the calculation. For example, a calculation with an N of 64 may take less than a second in certain setting, whereas an N of 128 may take ˜7.5 seconds using the same setting. Thus focusing the grid size down enables the calculation to maintain a high degree of rotational and translational resolution.

The Fourier Correlation Algorithm

The Fourier correlation algorithm (FCA) relies on the fast Fourier transform to scan the translational space of two rigidly rotating geometric shapes much more rapidly. To obtain the correlation between molecules a and b as a function of translation, the above discrete functions which represent each molecule (a and b) are first Fourier transformed (denoted DFT for discrete fast Fourier transform) according to, for example, Elliott and Rao (in Fast Transforms: Algorithms, Analysis, Applications, pp58-90, 1982. Academic Press, Orlando, Fla. Entire content of which is incorporated herein by reference).

Briefly, the DFT of a function x_{l,m,n} is defined as:

$${X}_{o , p , q} = {\sum }_{l = 1}^{N} {\sum }_{m = 1}^{N} {\sum }_{n = 1}^{N} exp [ - 2 π i ( ol + pm + qn ) / N ] \times {x}_{l , m , n} , \qquad [ Eq . 3 ]$$

where o, p, q={1 . . . N} and i=\sqrt{−1}.

When applying this transformation to both sides of Eq. 2 (see Papoulis, in The Fourier Integral and its Applications, MacGraw-Hill, New York, pp.244-245, 1962. Incorporated herein by reference), it yields:

C_{o,p,q}=A*_{o,p,q}×B_{o,p,q} [Eq. 4]

Where C and B are the DFT of the functions c and b, respectively, of Eq. 2; and A* is the complex conjugate of the DFT the function a in Eq. 2.

Eq. 4 indicates that the transformed correlation function C is obtained by a simple multiplication of the two functions A* and B. The Inverse Fourier transform (IFT) (see Elliott and Rao, supra), defined as

$${c}_{α , β , γ} = 1 / {N}^{3} {\sum }_{l = 1}^{N} {\sum }_{m = 1}^{N} {\sum }_{n = 1}^{N} exp [ - 2 π i ( ol + pm + qn ) / N ] \times {C}_{o , p , q} , \qquad [ Eq . 5 ]$$

is used to obtain the desired correlation between the two original functions a and b.

The application of the FCA reduces the number of translational calculations (i.e. ˜N⁶) down to the order of N³ ln(N³) (Press et al, 1986 and Bracewell, 1990). Use of this method reduces the computational complexity while maintaining a high degree of translational resolution.

After each translational scan, molecule a is fixed, while molecule b is rotated about one of its Euler angles until rotational space has been completely scanned. To illustrate, for an angular deviation of 60 =15°, this yields 360×360×180/α³=6912 orientations. However, many of these orientations are degenerate and must be removed using the following relationship (Lattman, 1972, In The Molecular Replacement Method, pp. 179-185, Gordon and Breach, New York, Rossman, M. G., ed.):

α=cos⁻ⁱ{[tr(R₁×R₂^{T})−1]/2} [Eq. 6]

where R₁ is the rotation matrix of the first orientation, R₂^{T} is the transpose of the rotation matrix of the second orientation, and tr is the matrix trace. If α≦1°, then the two orientations are degenerate. Removing degeneracies in this fashion yields 6385 unique orientations out of a possible 6912 if α is 15°. A finer angular rotation is more computationally demanding for extensive trials, but can nonetheless be achieved using faster computers and/or longer calculation times.

The entire procedure described above can be summarized by the following steps:

(i) derive a_{l,m,n} from atomic coordinates of molecule a (Eq. 2),

(ii) A*=[DFT(a_{l,m,n})]* (Eq. 4),

(iii) derive b_{l,m,n} from atomic coordinates of molecule b (Eq. 2),

(iv) B=DFT(b_{l,m,n}) (Eq. 4),

(v) C=A*·B (Eq. 5),

(vi) c=IFT(C) (Eq. 6),

(vii) look for a sharp positive peak of c

(viii) rotate molecule b to a new orientation,

(ix) repeat steps iii-viii and end when the orientations scan is completed, and

(x) sort all of the peaks by their height.

Each high and sharp peak found by this procedure indicates geometric match and thus represents a potential complex. The relative position and orientation of the molecules within each such complex can readily be derived from the coordinates of the correlation peak, and from the three Euler angles at which the peak was found.

To illustrate, an exemplary implementation of the algorithm is described below by assigning specific values to the various parameters involved—i.e., the surface layer thickness, r, Δ, ρ, δ, N, and the grid step size denoted by η. The choice of these values is based on a number of considerations, outlined in this section. However, it should be understood that these examples are by no means limiting. Other similar parameters may be used depending on specific needs.

Since the match between the functions a and b may not be perfect, because, for example, the structure of known complexes reveals small gaps between the molecules, which are also reflected in their mathematical representation. To ensure that the correct match between molecules is not missed, our algorithm must be able to tolerate these imperfections. This is achieved by assigning more than one layer of grid points to the surface in a so that the surface thickness for molecule a is 1.5-2.5 Å. Consequently, penetrations and gaps that are smaller than these values are tolerated. It should be noted that an inherent drawback in the choice of a thicker surface layer is the concomitant increase in the number of faulty matches.

The thickness of the surface layer also influences the angular tolerance. This tolerance is defined as the maximal deviation from the correct match orientation that would still result in a distinct correlation peak. Typically, a surface layer thickness of 2 Å yielded an angular tolerance of about ±10°. Thus, the angular step Δ was set to 20°, resulting in 2916 different orientations of molecule b at each of which the correlation function had to be evaluated.

The parameter r, used to derive the functions a_{l,m,n} and b_{l,m,n}, can be set to 1.8 Å, which is larger by about 0.2 Å than the average van der Waals radius for carbon, nitrogen, and oxygen. Although a range of usable r values can be employed, such as 1-3 Å, preferably 1.4-2.5 Å, most preferably 1.6-1.8 Å for molecule a. The r value is generally about 0.2-0.5 Å larger for molecule b in order to compensate for stripping if appropriate.

The parameters ρ and δ, representing the interior of the molecules, may be set to −15 and 1, respectively. This ensures that the correlation value is substantially reduced in case of penetration. Several other choices for ρ and δ, in the ranges ρ<<−1 and 0≦δ≦1, may not significantly affect the performance of the algorithm.

Another important parameter of the algorithm is the grid step size, in. Optimal results have been obtained when η was set to 0.7-0.8 Å, corresponding to half of the carbon-carbon bond length. Yet, since the product η·N should be larger than the size of any potential complex, a finer grid requires a larger number of points N. This leads in turn to excessive computation time Therefore, it might be advantageous to perform an initial scan of the angular orientations with larger grid steps (η≡1.0-1.2 Å); thus, even with a slow computer, computations that would take days with the finer grid were performed in hours. However, with such large grid steps, spurious correlation peaks, which may even be higher than the correct peak, appear. Hence, the scan stage was followed by a discrimination stage, in which the correlation functions were recalculated with a finer grid (η≡0.7-0.8 Å), but only for those orientations that yielded the highest peaks in the scan stage. This discrimination stage will enhance the correct correlation peak and suppress spurious peaks.

A FORTRAN program may be used for implementing the algorithm. For example, the parameters of the program, in accordance with the arguments given above, can be assigned the following values: r=1.8 Å, Δ=20°, η=−15, δ=1, N=90 (η≡1.0-1.2 Å) for the scan stage, and N=128 (η≡0.7-0.8 Å) for the discrimination stage. The program may be run on a Convex C-220 computer with the Veclib fast Fourier transform subroutine, or any other equivalent computers. In one exemplary calculation using these specific settings, the computation time for each iteration (steps iii-viii in the summarized algorithm) in the scan stage was 9 sec. The total computation time for matching two molecules in the range of 1100 atoms each, including both the initial scan and the discrimination stage, was typically 7.5 hr. Faster computers are expected to significantly reduce the calculation time by 10-1000 folds. See Example below.

Low resolution grids and coarser rotational scans can be used for the initial search (see Katchalski-Katzir et al., 1992; Vakser & Aflalo, 1994; Meyer et al., 1996). Vakser & Aflalo (1994), for example, used a 64×64×64 grid with an angular deviation of 20° for the global search. However, certain systems, such as the Antibody/antigen system, may be too large to model at this grid resolution and angular deviation. Access to a more powerful computer capable of performing the FFT in parallel may at least partially solve this problem by enabling rapid docking involving both stages of the search (i.e. global search and local refinement) at high resolution.

A thinner surface layer may also be advantageous in certain cases. A thinner surface layer demands greater shape specificity and previous results show that a surface thickness of 1.5 Å works well when docking unbound proteins. Decreasing surface thickness to 1.2 Å during local refinement improved results even further. Local refinement using the same surface thickness (i.e. 1.5 Å) as the global search may less able to distinguish correctly docked molecules clearly. This may suggest that a sufficient level of surface complementarity could still exist at the protein-protein interface in spite of incorrectly positioned side-chains.

Successful docking process of the potentially interacting molecules may be enhanced by performing one or more of the following additional calculations which take into consideration of non-geometric factors such as electrostatic forces and/or available biological information.

i) Measuring Electrostatic Complementarity by Fourier Correlation

In certain situations, shape complementarity may not be the only factor involved in molecular binding. Electrostatic attraction, particularly the specific charge-charge interactions in the binding interface, also plays an important role. For speed and consistency, electrostatic complementarity can be calculated by Fourier correlation using a simple Coulombic model. Since charged amino acid side-chains are usually on the protein surface, they are often involved in binding and tend to be highly flexible. Therefore, calculating individual point charge interactions when attempting to dock the uncomplexed whole structures may not be feasible and can produce misleading results. So rather than try to measure specific charge-charge interactions, the point charges of one protein interacting with the electric field of the other as grid points can be measured. In this way, point charges are dispersed to simulate side-chain movement. (However, alternative methods that calculates individual point charge interactions may also be used in the instant method since all or most side-chain atoms are removed from the atomic coordinates).

Although the calculation presented below uses a simple Coulombic model, a more rigorous, and more computationally expensive calculation model, such as the Poisson-Boltzmann description (Warwicker & Watson, 1982; Sharp & Honig, 1990) may also be used, especially when the side-chain coordinates are removed.

The electrostatic calculations proceed in a manner very similar to those of shape complementarity. Charges are assigned to the atoms of molecule a (Table 1) and the molecule is placed in a grid. The electric field at each grid node (excluding those of the protein core) is calculated:

$${φ}_{i} = {\sum }_{j} \{ {q}_{j} / [ ϵ ( {r}_{ij} ) {r}_{ij} ] \} \qquad [ Eq . 7 ]$$

where φᵢ, is the field strength at node i (position l,m,n), qⱼ is the charge on atomj, rᵢⱼ is the distance between i and j (a minimum cutoff distance of 2 Å can be imposed to avoid artificially large values of φ), and ε(rᵢⱼ) is a distance-dependent dielectric function. In this case, a pseudo-sigmoidal function, based on the sigmoidal function of Hingerty et al. (1985), is used:

ε(rᵢⱼ)=(I)4, if rᵢⱼ≦6 Å; (II) 38rᵢⱼ−224, if 6 Å<rᵢⱼ<8 Å; or (III) 80, if rᵢⱼ>8 Å. [Eq. 8]

Several distance-dependent dielectric functions have been tested. This one was chosen because it effectively damps long-range electrostatic effects that are not relevant to the binding interface. In fact, dielectric functions that do not damp long-range effects give inconsistent results, sometimes showing poor electrostatics for experimentally determined complexes. The treatment of molecule b is much simpler. Charges are assigned to its atoms and then discretized in a grid (q_{l,m,n}) by trilinear weighting (Rogers & Sternberg, 1984; Edmonds et al., 1984). Calculations of the electrostatic interactions proceeds as outlined and as described for surface correlation except that the discrete functions are:

A_{l,m,n}=(I)φ_{l,m,n}, for entire grid excluding core; and (II) 0, for core of molecule.

B_{l,m,n}=q_{l,m,n}

and the correlation function becomes:

f_{α,β,γ}^{elec}=A_{l,m,n}×qB_{l+α,m+β,n+γ}

So both grids are Fourier transformed and correlated such that the static charges of molecule b move through the electric field of molecule a. The electrostatic correlation score is used as a binary filter. Specifically, false positive geometries that give high shape correlation scores' can be excluded if their electrostatic correlation is unfavorable (i.e. positive).

TABLE 1
Charges that can be used in Coulombic electrostatic fields

| Peptide Backbone | Charge | Side-Chain Atoms | Charges |
|---|---|---|---|
| Terminal-N | 1.0 | Arg-N^{η} | 0.5 |
| Terrninal-O | −1.0 | Glu-O^{ε} | −0.5 |
| C^{α} | 0.0 | Asp-O^{δ} | −0.5 |
| C | 0.0 | Lys-N^{ζ} | 1.0 |
| O | −0.5 | Pro-N | −0.1 |
| N | 0.5 | | |

Please note that in certain calculations, only backbone charges will be used if all side-chain atomic coordinates are stripped off.

The results of the global search before filtering and without consideration of electrostatics show that geometric complementarity alone (as measured by Katchalski-Katzir et al., 1992) may not always reliably dock unbound complexes. A high surface correlation score does not always indicate a correctly docked complex. For example, even a limited rotational scan near the correct geometry produces a broad range of correlation scores. In a complete rotational search it is possible to find incorrectly docked complexes that score higher than the actual crystal structure. This does not, however, pose a problem because the aim during the global search is to place at least one near-correct prediction in the final output; not necessarily at the highest scoring position. Correctly docked complexes can be screened later using experimental constraints and advanced refinement techniques.

The additional constraint of removing predictions with unfavorable electrostatic interactions may markedly improve the ranking of correctly docked structures in the global search. With electrostatics, a good solution is almost always found in the top 4000 structures. In general, inclusion of electrostatics reduces the number of geometries to be evaluated by approximately 50%. However, at least in one embodiment of the invention, calculations involving any non-geometric considerations are explicitly excluded.

ii) Filterin Based on Biological Information

Knowledge of the location of the binding site on one, or both proteins may drastically reduce the number of possible allowed conformations. Knowing specific binding site residues reduces the search space even further. It is possible to utilize this information in the form of distance constraints. Generally, information about the binding site is available from experimental data (e.g. site-directed mutagenesis, chemical cross-linking, phylogenetic data, etc.). In the absence of experimental data, it is often possible to predict the correct binding site by examining potential hydrogen bonding groups, clefts and/or charged sites on a protein surface (Gilson & Honig, 1987; Desjarlais et al., 1988; Nicholls & Honig 1991; Laskowski, 1995; Laskowski et al., 1996; Meyer et al., 1996). For example, immunoglobulin represent a system where the binding sites are known in advance. The complementarity determining region (CDR) of immunoglobulins are well characterized. This information can be used to varying degrees in the docking experiments. For example, in an enzyme-inhibitor model, filters can be defined as: loose, any residue of the inhibitor in contact with any residue of the enzyme active site; medium, an inhibitor residue in contact with certain of the catalytic residues; tight, a specific binding site residue of the inhibitor in contact with the catalytic residues. In the antibody/antigen docking attempts, filters can be defined as: loose, any part of the antigen in contact with either the L3 or the H3 CDR; medium, antigen in contact with both the L3 and H3 CDRs; tight, the medium filter together with one residue of the epitope in contact with any part of the CDR. The L3/H3 CDR filters are based on the study of MacCullum et al. (1996), which analyzed general structural principles of antibody/antigen contacts.

If the proteins in question are heavily studied, as immunoglobulins, information typical of the medium or the tight filter might be available. In other practical applications of docking, there often will be some available constraints typical of the loose filter. Typically, loose filtering of the output from the global search removes between 79 and 99% of false positives, usually leaving at least one correct answer in the top 50 predictions. Furthermore, medium and tight filtering may successively remove more incorrect predictions. In many cases, going from loose to medium filtering reduces the total number of predictions by an order of magnitude. With fewer false positives to contend with, the ranking of the correct predictions improves dramatically. In going from medium to tight filtering, although the procedure does not significantly alter the number of false positives, a near correct geometry is ranked in the top five predictions in almost all cases. It is clear that given experimental constraints this docking procedure can effectively dock two proteins using crystal structures for the unbound subunits. Since few predictions remain at this stage, it may be possible to use more computationally demanding, and hence more accurate models to predict binding.

To illustrate further, distance filtering can be implemented as a two-step process. First, a rapid check of intermolecular Cα distances between constraint residues is performed. For example, in the case of an antibody-antigen binding where the epitope is unknown, the C^{α} distances between residues in the hypervariable loops and all antigen residues would be checked. If a pair of C^{α} atoms is within a cutoff distance, the distances between all atoms of the two residues are checked. If any atom pair is within a specific distance, for example, 4.5 Å, then the distance constraint is satisfied. Predicted complexes that do not satisfy the distance constraint can be discarded.

iii) Local Refinement of Predicted Geometries

In a typical initial global search, the angular deviation at is set at 15°. A finer rotational scan is desirable but computationally expensive. So, a local refinement of the most reasonable predictions can be performed. For example, structures that have passed through the loose filter can be chosen for further refinement because this level of information is generally available. During illustrative refinement, each geometry is shifted (+5 Å in each direction) and rotated (±5° for each Euler angle) slightly to find the highest surface correlation score in the local space. Refinement may use the same surface thickness as in the global search (1.5 Å). However, a thinner surface thickness (such as 1.2 Å) may also be used, which is generally less tolerant of overlapping protein surfaces. This leads to more stringent scoring of shape complementarity, something that is not necessarily beneficial when doing a global search of docking space for native structures. However, stricter shape fitting tends to dampen correlation scores for false positives while enhancing those of predicted complexes already near the correct docking geometry. Generally, using a slightly thinner surface thickness significantly improves ranking.

To illustrate, a complete docking experiment may consists of two distinct phases: global search and local refinement. It is possible that in certain embodiments, high-resolution grids are used in both phases, while in certain other embodiments, smaller, low-resolution grids are used during the global search. The availability of high speed multiprocessing using faster computers makes it possible to use a high-resolution grid for both the initial search and the refinement.

In an exemplary search, the global search examines all translation (i.e. within the discrete space of the grid) and rotation space, and produces (128³×6385)≡10¹⁰ possible docking geometries. Obviously, geometries with zero or negative correlation scores can be excluded immediately. However, several hundred thousand possible docking geometries still remain. To reduce the number of possibilities to manageable levels, only three complexes from each translational scan may need to be saved to the “stack”; those with the highest surface correlation scores and favorable electrostatics. This leaves (3×6385) 19,155 possible complexes after the global search. The stack is sorted and the best 4000 geometries are kept. These complexes are filtered on the basis of available biochemical information and those that pass through the filter undergo local refinement. Each predicted geometry is shifted (±5 Å in each direction) and rotated (±5° for each Euler angle) slightly and the highest scoring complex is saved. Electrostatics may not need to be calculated during local refinement for two reasons. First, it doubles the computational time required for the refinement stage of docking. Second, the docked geometry may not change significantly during refinement. Consequently, electrostatic interaction will not change significantly. At the end of the local refinement stage of docking, the stack may be filtered again and the remaining complexes with the highest surface correlation scores are considered reasonable docking predictions.

A similar complete docking package used in Gabb et al. (supra), named FTDOCK, consists of approximately 3,500 lines of Fortran 77 and Perl 5.0 (Wall & Schwartz, 1991) code designed to run under the UNIX operating system. In that study, all docking experiments were carried out on an SGI Power Challenge symmetric-array multiprocessor with 12 RI 0000 CPUs. Parallel-compiler directives as well as the LIBFFT parallel maths library (J.-P. Panziera, SGI Paris, France) containing the necessary FFT routines are used to maximize computational efficiency. A complete docking experiment including post-filtering requires approximately six hours of CPU time using eight processors simultaneously. Preprocessor commands in the source code allow compilation on serial workstations. Similar configurations may also be used in the instant invention.

D. Interfacial Side-Chain Selection with the ORBIT Suite of Design Algorithms (Step 2)

The primary function of the ORBIT algorithms is to return an optimal (candidate) protein sequence for a given three-dimensional structure (Street and Mayo, 1999, also see Xencor, Inc. website and U.S. Pat. Nos. 6,514,729; 6,403,312; 6,269,312; and 6,188,965, all incorporated herein by reference). They do so by employing an unbiased, quantitative design method based on the physical chemical properties that determine protein structure and stability. The combined algorithms provide tools for defining a backbone structure, classifying residues into core, boundary and surface categories, selecting the optimal sequence and arrangement of amino acids, and analyzing the energies of the predicted structures. The entire suite of algorithms are utilized in this second step of the docking algorithm (i.e. repacking of the interfacial residues).

The atomic coordinates of the docked orientation that exhibits the highest protein/protein surface shape complementarity are modified and subsequently treated as those of a single protein. The modified “pseudo single protein” coordinates are fed into the ORBIT design algorithms where the interfacial residues are reclassified as buried core residues.

One of the ORBIT algorithms, RESLASS, which classifies residues as core, boundary or surface based on their position in a protein, is used to determine which residues become buried (i.e. change classification) upon protein docking (e.g. residues that reclassify from surface to core, boundary to core or surface to boundary). To accomplish this, the residues of each monomer are classified first in the context of the free proteins and then in that of the docked complex. Residues which change classification upon complex formation (i.e. become buried) are then targeted for computational side-chain selection by other ORBIT design algorithms (e.g. SETUP and DEE). Residues which are reclassified as core are substituted with hydrophobic side-chains while those reclassified from surface to boundary are substituted primarily with hydrophilic side-chains. Additionally, residues which do not change classification upon complex formation but are in close enough proximity to form favorable intermolecular interactions are also targeted for side-chain selection. These residues are substituted primarily with hydrophilic side-chains.

i) ORBIT Design Calculations

The protein design algorithm ORBIT, described in Dahiyat and Mayo (Protein Sci 5(5): 895-903, 1996; and Science 278: 82-87, 1997, entire contents incorporated herein by reference) and Dahiyat et al. (J Mol Biol 273(4): 789-96, 1997, entire content incorporated herein by reference) can be used to predict the optimal amino acid sequences of the binding pocket for binding to the different analogs. Although other similar or equivalent algorithms may also be used for the same purpose with minor modification. Selection of amino acids is performed using a very efficient DEE (Dead-End Elimination)-related search algorithm (see below) that relies on a discrete set of allowed conformations (“rotamers”) for each side-chain and empirical potential energy functions that are used to calculate pair-wise interactions between side-chains and the between the side-chains and backbone.

Surveys of protein structure database have shown that side-chains exhibit marked conformational preferences, and that most side-chains are limited to a small number of torsional angles. Thus, the torsional flexibility of most amino acids can be represented with a discrete set of allowed conformations called rotamers. Backbone-dependent rotameric preferences in side-chains are observed in crystal structures, based on the dependency of the rotamers on the main-chain conformations. ORBIT accounts for the torsional flexibilities of side-chains by providing rotamer libraries that are based on those developed by Dunbrack and Karplus (Dunbrack and Karplus, J Mol Biol 230(2): 543-74, 1993; Dunbrack and Karplus, Nat Struct Biol 1(5): 334-40, 1994, entire contents of which are all incorporated herein by reference).

In our design, we would like to optimize the binding interface of the candidate and target molecules for binding to each other. In performing the optimization calculations, we would like to vary the torsional angles of the intended analogs and side-chains lining the pocket simultaneously. This requires generating rotamer libraries for the analogs, since they are not included in the standard rotamer libraries. Backbone independent rotamer libraries for all the analogs are generated as described below.

Since the residues in the pocket are buried in the protein structure, we used force field parameters similar to those used in protein core design calculations. The design algorithm uses energy terms based on a force field that includes van der Waals interactions, electrostatic interactions, hydrogen bonding, and solvation effects (see Gordon et al., Curr Opin Struct Biol 9(4): 509-13, 1999, entire content incorporated herein by reference).

Based on the crystallographic data, residue positions in the interface or near the interface are identified. These residue positions are potential target positions for redesign.

Design calculations (see below) are run by fixing the identity of all other residues, while varying the target positions on the interface residues described above. Certain target positions may be allowed to be any of the 20 natural amino acids, with the possible exception of proline, methionine or cysteine. These amino acids may nevertheless by be allowed at those positions if the wild-type identity of these positions are Met, Pro, or Cys. At certain other target positions, only amino acids with a certain characteristic (such as small, large, hydrophobic, hydrophilic, aromatic, etc.) are allowed based on the need of the design. It is expected that many of these target positions are buried in the core and a number of them may pack against the natural substrate in the crystal structure.

Information from other independent studies, such as mutation analysis at a specific position which has been shown to have altered substrate specificity may also help in the design process.

As described above, residues not involved in interface interaction may be held fixed both in identity and conformation in all the calculations. These residues probably do not contribute to the interaction of the molecules directly.

In one embodiment, all the side-chain rotamers generated in any rotamer library are allowed in the calculation. Alternatively, calculation(s) can be run allowing only those backbone-dependent rotamers in the binding interface. These are the rotamers with all possible combinations of χ1 and χ2 of the natural interface amino acid with a maximal of ±20° of torsional angle variations, in increments of, say 1°, 2°, 3°, or 5°, etc. The structure generated in this calculation preferably will have a tightly packed interface between the candidate and target molecules. If only the candidate polypeptide is to be redesigned, the target polypeptide are not allowed to change side-chain amino acid identities, but only different rotamers of the fixed amino acids; the candidate polypeptide interface residues can change both identity and rotameric conformations. If both the candidate and target polypeptides are to be redesigned, then all interfacial residues can change identity (non-wild-type sequence) and rotameric conformations.

The present invention utilizes an “inverse protein folding” approach directed to the quantitative design and optimization of amino acid sequences, especially the candidate (and optional target proteins) bound through the interface identified based on geometry (or other non-geometric factors). Similar to protein design, such approach seeks to find a sequence or set of sequences that will fold into a desired structure. These approaches can be contrasted with a “protein folding” approach which attempts to predict a structure taken by a given sequence. In a generalized approach, target varying residue positions that is selected for redesign are determined based on the criteria described above. Each variable residue position can then be reclassified as a core residue, a surface residue, or a boundary residue. In that case, each classification defines a subset of possible amino acid residues for the position (for example, core residues generally will be selected from the set of hydrophobic residues, surface residues generally will be selected from the hydrophilic residues, and boundary residues may be either). Each amino acid residue can be represented by a discrete set of all allowed conformers of each side-chain, called rotamers. Thus, to arrive at an optimal sequence for a binding interface, all possible sequences of rotamers or a specific subset thereof may be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side-chain with all or part of the backbone (the “singles” energy, also called the rotamer/template or rotamer/backbone energy), and the interaction of the rotamer side-chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics (see Gordon et al., supra). Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a search (such as Monte Carlo search) may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated. Typically, 10⁶ jumps (steps) are used in a Monte Carlo search.

The results may then be experimentally verified by physically generating one or more of the protein sequences followed by experimental testing. The information obtained from the testing can then be fed back into the analysis, to modify the procedure if necessary.

ii) Rotamers for Target (Varying Posision Residues

Once the pseudo protein (complex) backbone structure (including the backbone structure of two or more docketed proteins, etc.)) has been selected and input, and the variable residue positions chosen, a group of potential rotamers for each of the variable residue positions is established.

As is known in the art, each amino acid side-chain has a set of possible conformers, called rotamers. See Ponder, et al., Acad. Press Inc. (London) Ltd. pp. 775-791 (1987); Dunbrack, et al., Struc. Biol. 1(5): 334-340 (1994); Desmet, et al., Nature 356: 539-542 (1992), all of which are hereby expressly incorporated by reference in their entirety. Thus, a set of discrete rotamers for every amino acid side-chain is used. As described above, there are two general types of rotamer libraries: backbone dependent and backbone independent. Either type of library can be used at any position.

In addition, a preferred embodiment does a type of “fine tuning” of the rotamer library by expanding the possible χ angle values of the rotamers by plus and minus one standard deviation (±1 SD) (or more) about the mean value, in order to minimize possible errors that might arise from the discreteness of the library. This is particularly important for aromatic residues, and fairly important for hydrophobic residues, due to the increased requirements for flexibility in the core and the rigidity of aromatic rings; it is not as important for the other residues. Thus a preferred embodiment expands the χ1 and χ² angles for all amino acids except Met, Arg and Lys. For the intended amino acid analogs, the χ1 and χ2 angles are expanded as such in their corresponding rotamers.

To roughly illustrate the numbers of rotamers, in one version of the Dunbrack & Karplus backbone-dependent rotamer library, Ala has 1 rotamer, Gly has 1 rotamer, Arg has 55 rotamers, The has 9 rotamers, Lys has 57 rotamers, Glu has 69 rotamers, Asn has 54 rotamers, Asp has 27 rotamers, Trp has 54 rotamers, Tyr has 36 rotamers, Cys has 9 rotamers, Gln has 69 rotamers, His has 54 rotamers, Val has 9 rotamers, Ile has 45 rotamers, Leu has 36 rotamers, Mat has 21 rotamers, Ser has 9 rotamers, and Phe has 36 rotamers.

In general, proline is not generally used in a target position, since it will rarely be chosen for any position, although it can be included if desired. Similarly, a preferred embodiment omits cysteine as a consideration, only to avoid potential disulfide problems, although it can be included if desired.

As will be appreciated by those in the art, other rotamer libraries with all dihedral angles staggered can be used or generated.

In a preferred embodiment, at a minimum, at least one variable position has rotamers from at least two different amino acid side-chains; that is, a sequence is being optimized, rather than a structure.

In a preferred embodiment, rotamers from all of the amino acids (or all of them except cysteine, glycine and proline) are used for each variable residue position; that is, the group or set of potential rotamers at each variable position is every possible rotamer of each amino acid. This is especially preferred when the number of variable positions is not high as this type of analysis can be computationally expensive.

iii) Determining Conformational Energy

In certain embodiments, each variable position is classified as either a core, surface or boundary residue position, although in some cases, as explained below, the variable position may be set to glycine to minimize backbone strain.

The classification of residue positions as core, surface or boundary may be done in several ways, as will be appreciated by those in the art. In a preferred embodiment, the classification is done via a visual scan of the original protein backbone structure, including the side-chains, and assigning a classification based on a subjective evaluation of one skilled in the art of protein modeling. Alternatively, a preferred embodiment utilizes an assessment of the orientation of the C_{α}-C_{β} vectors relative to a solvent accessible surface computed using only the template C_{α} atoms. In a preferred embodiment, the solvent accessible surface for only the C_{α} atoms of the target fold is generated using the Connolly algorithm with a probe radius ranging from about 4 to about 12 Å, with from about 6 to about 10 Å being preferred, and 8 Å being particularly preferred. The C_{α} radius used ranges from about 1.6 Å to about 2.3 Å, with from about 1.8 to about 2.1 Å being preferred, and 1.95 Å being especially preferred. A residue is classified as a core position if a) the distance for its C_{α}, along its C_{α}-C_{β} vector, to the solvent accessible surface is greater than about 4-6 Å, with greater than about 5.0 Å being especially preferred, and b) the distance for its C_{β} to the nearest surface point is greater than about 1.5-3 Å, with greater than about 2.0 Å being especially preferred. The remaining residues are classified as surface positions if the sum of the distances from their C_{α}, along their C_{α}-C_{β} vector, to the solvent accessible surface, plus the distance from their C_{β} to the closest surface point was less than about 2.5-4 Å., with less than about 2.7 Å being especially preferred. All remaining residues are classified as boundary positions. For example, residues in the binding pocket are buried in the protein structure, force field parameters similar to those used in protein core design calculations can be used when calculating these residues.

Once each variable position is classified as either core, surface or boundary, a set of amino acid side-chains, and thus a set of rotamers, is assigned to each position. That is, the set of possible amino acid side-chains that the program will allow to be considered at any particular position is chosen. Subsequently, once the possible amino acid side-chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side-chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be).

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side-chains, and thus the rotamers for these side-chains are not used. However, in a preferred embodiment, when the variable residue position has a Φangle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once a three-dimensional structure has been obtained or otherwise provided for the AARS sequence as described above, a fitness value for the protein may be obtained by calculating or determining the “conformational energy” or “energy” E of the protein structure. In particular and without being limited to any particular theory or mechanism of action, sequences that have a lower (i.e., more negative) conformational energy are typically expected to be more stable and therefore more “fit” than are sequences having higher (i.e., less negative) conformation energy. Thus, the fitness of a sequence is preferably related to its negative conformational energy; i.e., F=−E.

Typically, the conformational energy is calculated ab initio from the conformation determination discussed above, and using an empirical or semi-empirical force field such as CHARM (Brooks et al., J. Comp. Chem. 1983, 4:187-217; MacKerell et al., in The Encyclopedia of Computational Chemistry, Vol. 1:271-277, John Wiley & Sons, Chichester, 1998) AMBER (see, Cornell et al., J. Amer Chem. Soc. 1995, 117:5179; Woods et al., J. Phys. Chem. 1995,99:3832-3846; Weineretal., J. Comp. Chem. 1986,7:230; and Weiner et al., J. Amer. Chem. Soc. 1984, 106:765) and DREIDING (Mayo et al., J. Phys. Chem. 1990, 94:8897) to name a few. These and other such force-fields comprise a number of potential scoring functions and parameters for at least approximate contributions of various interactions within a macromolecule.

The scoring functions include a van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:

Eₜₒₜₐₗ=nE_{vdw}+nEₐₛ+nE_{h-bonding}+nEₛₛ+nE_{elec} (Equation 1)

In Equation 1, the total energy is the sum of the energy of the van der Waals potential (E_{vdW}), the energy of atomic solvation (Eₐₛ) the energy of hydrogen bonding (E_{h-bonding}), the energy of secondary structure (Eₛₛ) and the energy of electrostatic interaction (E_{elec}). The term n is either 0 or 1, depending on whether the term is to be considered for the particular residue position, as is more fully outlined below.

In a preferred embodiment, a van der Waals' scoring function is used. As is known in the art, van der Waals' forces are the weak, non-covalent and non-ionic forces between atoms and molecules, that is, the induced dipole and electron repulsion (Pauli principle) forces. The van der Waals scoring function is based on a van der Waals potential energy. There are a number of van der Waals potential energy calculations, including a Lennard-Jones 12-6 potential with radii and well depth parameters from the Dreiding force field, Mayo et al., J. Prot. Chem., 1990, expressly incorporated herein by reference, or the exponential 6 potential. Equation 2, shown below, is the preferred Lennard-Jones potential:

E_{vdw}=D₀{(R₀/R)¹²−2(R₀/R)⁶} (Equation 2)

R₀ is the geometric mean of the van der Waals radii of the two atoms under consideration, and D₀ is the geometric mean of the well depth of the two atoms under consideration. E_{vdw} and R are the energy and interatomic distance between the two atoms under consideration, as is more fully described below.

In a preferred embodiment, the van der Waals forces are scaled using a scaling factor, a. Equation 3 shows the use of α in the van der Waals Lennard-Jones potential equation:

E_{vdw}=D₀{(αR₀/R)¹²−2(αR₀/R)⁶} (Equation 3)

The role of the a scaling factor is to change the importance of packing effects in the optimization and design of any particular protein. As discussed in the Examples, different values for α result in different sequences being generated by the present methods. Specifically, a reduced van der Waals steric constraint can compensate for the restrictive effect of a fixed backbone and discrete side-chain rotamers in the simulation and can allow a broader sampling of sequences compatible with a desired fold. In a preferred embodiment, α values ranging from about 0.70 to about 1.10 can be used, with α values from about 0.8 to about 1.05 being preferred, and from about 0.85 to about 1.0 being especially preferred. Specific α values which are preferred are 0.80, 0.85, 0.90, 0.95, 1.00, and 1.05.

Generally speaking, variation of the van der Waals scale factor α results in four regimes of packing specificity: regime 1 where 0.9<=α<=1.05 and packing constraints dominate the sequence selection; regime 2 where 0.8<=α<0.9 and the hydrophobic solvation potential begins to compete with packing forces; regime 3 where α<0.8 and hydrophobic solvation dominates the design; and, regime 4 where α>1.05 and van der Waals repulsions appear to be too severe to allow meaningful sequence selection. In particular, different a values may be used for core, surface and boundary positions, with regimes 1 and 2 being preferred for core residues, regime 1 being preferred for surface residues, and regime 1 and 2 being preferred for boundary residues.

In a preferred embodiment, the van der Waals scaling factor is used in the total energy calculations for each variable residue position, including core, surface and boundary positions.

In a preferred embodiment, an atomic solvation potential scoring function is used. As is appreciated by those in the art, solvent interactions of a protein are a significant factor in protein stability, and residue/protein hydrophobicity has been shown to be the major driving force in protein folding. Thus, there is an entropic cost to solvating hydrophobic surfaces, in addition to the potential for misfolding or aggregation. Accordingly, the burial of hydrophobic surfaces within a protein structure is beneficial to both folding and stability. Similarly, there can be a disadvantage for burying hydrophilic residues. The accessible surface area of a protein atom is generally defined as the area of the surface over which a water molecule can be placed while making van der Waals contact with this atom and not penetrating any other protein atom. Thus, in a preferred embodiment, the salvation potential is generally scored by taking the total possible exposed surface area of the moiety or two independent moieties (either a rotamer or the first rotamer and the second rotamer), which is the reference, and subtracting out the “buried” area, i.e. the area which is not solvent exposed due to interactions either with the backbone or with other rotamers. This thus gives the exposed surface area.

Alternatively, a preferred embodiment calculates the scoring function on the basis of the “buried” portion; i.e. the total possible exposed surface area is calculated, and then the calculated surface area after the interaction of the moieties is subtracted, leaving the buried surface area. A particularly preferred method does both of these calculations.

As is more fully described below, both of these methods can be done in a variety of ways. See Eisenberg et al., Nature 319:199-203 (1986); Connolly, Science 221:709-713 (1983); and Wodak, et al., Proc. Natl. Acad. Sci. USA 77(4):1736-1740 (1980), all of which are expressly incorporated herein by reference. As will be appreciated by those in the art, this solvation potential scoring function is conformation dependent, rather than conformation independent.

In a preferred embodiment, the pair-wise salvation potential is implemented in two components, “singles” (rotamer/template) and “doubles” (rotamer/rotamer), as is more fully described below. For the rotamer/template buried area, the reference state is defined as the rotamer in question at residue position i with the backbone atoms only of residues i−1, i and i+1, although in some instances just i may be used. Thus, in a preferred embodiment, the salvation potential is not calculated for the interaction of each backbone atom with a particular rotamer, although more may be done as required. The area of the side-chain is calculated with the backbone atoms excluding solvent but not counted in the area. The folded state is defined as the area of the rotamer in question at residue i, but now in the context of the entire template structure including non-optimized side-chains, i.e. every other foxed position residue. The rotamer/template buried area is the difference between the reference and the folded states. The rotamer/rotamer reference area can be done in two ways; one by using simply the sum of the areas of the isolated rotamers; the second includes the full backbone. The folded state is the area of the two rotamers placed in their relative positions on the protein scaffold but with no template atoms present. In a preferred embodiment, the Richards definition of solvent accessible surface area (Lee and Richards, J. Mol. Biol. 55:379-400, 1971, hereby incorporated by reference) is used, with a probe radius ranging from 0.8 to 1.6 Å, with 1.4 Å being preferred, and Drieding van der Waals radii, scaled from 0.8 to 1.0. Carbon and sulfur, and all attached hydrogens, are considered nonpolar. Nitrogen and oxygen, and all attached hydrogens, are considered polar. Surface areas are calculated with the Connolly algorithm using a dot density of 10 Å-2 (Connolly, (1983) (supra), hereby incorporated by reference).

In a preferred embodiment, there is a correction for a possible overestimation of buried surface area which may exist in the calculation of the energy of interaction between two rotamers (but not the interaction of a rotamer with the backbone). Since, as is generally outlined below, rotamers are only considered in pairs, that is, a first rotamer is only compared to a second rotamer during the “doubles” calculations, this may overestimate the amount of buried surface area in locations where more than two rotamers interact, that is, where rotamers from three or more residue positions come together. Thus, a correction or scaling factor is used as outlined below. The general energy of solvation is shown in Equation 4:

Eₛₐ=f(SA) (Equation 4)

where Eₛₐ is the energy of solvation, f is a constant used to correlate surface area and energy, and SA is the surface area. This equation can be broken down, depending on which parameter is being evaluated. Thus, when the hydrophobic buried surface area is used, Equation 5 is appropriate:

Eₛₐ=f₁(SA_{buried hydrophobic}) (Equation 5)

where f₁ is a constant which ranges from about 10 to about 50 cal/mol/Å², with 23 or 26 cal/mol/Å² being preferred. When a penalty for hydrophilic burial is being considered, the equation is shown in Equation 6:

Eₛₐ=f₁(SA_{buried hydrophobic})+f₂(SA_{buried hydrophilic}) (Equation 6)

where f₂ is a constant which ranges from −50 to −250 cal/mol/Å², with −86 or −100 cal/mol/Å² being preferred. Similarly, if a penalty for hydrophobic exposure is used, equation 7 or 8 may be used:

Eₛₐ=f₁(SA_{buried hydrophobic})+f₃(SA_{exposed hydrophobic}) (Equation 7)

Eₛₐ=f₁(SA_{buried hydrophobic})+f₂(SA_{buried hydrophilic})+f₃(SA_{exposed hydrophobic})+f₄(SA_{exposed hydrophilic}) (Equation 8)

In a preferred embodiment, f₃=−f₁.

In one embodiment, backbone atoms are not included in the calculation of surface areas, and values of 23 cal/mol/Å² (f₁) and −86 cal/mol/Å² (f₂) are determined.

In a preferred embodiment, this overcounting problem is addressed using a scaling factor that compensates for only the portion of the expression for pair-wise area that is subject to over-counting. In this embodiment, values of −26 cal/mol/Å² (f₁) and 100 cal/mol/Å² (f₂) are determined.

Atomic solvation energy is expensive, in terms of computational time and resources. Accordingly, in a preferred embodiment, the solvation energy is calculated for core and/or boundary residues, but not surface residues, with both a calculation for core and boundary residues being preferred, although any combination of the three is possible.

In a preferred embodiment, a hydrogen bond potential scoring function is used. A hydrogen bond potential is used as predicted hydrogen bonds do contribute to designed protein stability (see Stickle et al., J. Mol. Biol. 226:1143 (1992); Huyghues-Despointes et al., Biochem. 34:13267 (1995), both of which are expressly incorporated herein by reference). As outlined previously, explicit hydrogens are generated on the protein backbone structure.

In a preferred embodiment, the hydrogen bond potential consists of a distance-dependent term and an angle-dependent term, as shown in Equation 9:

E_{H-Bonding}=D₀{5(R₀/R)¹²−6(R₀/R)¹⁰}F(θ, φ, ψ) equation 9)

where R₀(2.8 Å) and D₀(8 kol/mol) are the hydrogen-bond equilibrium distance and well-depth, respectively, and R is the donor to acceptor distance. This hydrogen bond potential is based on the potential used in DREIDING with more restrictive angle-dependent terms to limit the occurrence of unfavorable hydrogen bond geometries. The angle term varies depending on the hybridization state of the donor and acceptor, as shown in Equations 10, 11, 12 and 13. Equation 10 is used for sp³ donor to sp³ acceptor; Equation 11 is used for sp³ donor to sp² acceptor, Equation 12 is used for sp² donor to sp³ acceptor, and Equation 13 is used for sp² donor to sp² acceptor:

F=cos² θcos²(φ−109.5) (Equation 10)

F=cos² θ cos² φ (Equation 11)

F=cos⁴ θ (Equation 12)

F=cos² θ cos²(max[(Φ, φ]) (Equation 13)

In Equations 10-13, θ is the donor-hydrogen-acceptor angle, φ is the hydrogen-acceptor-base angle (the base is the atom attached to the acceptor, for example the carbonyl carbon is the base for a carbonyl oxygen acceptor), and Φ is the angle between the normals of the planes defined by the six atoms attached to the sp² centers (the supplement of φ is used when φ is less than 90°). The hydrogen-bond function is only evaluated when 2.6 Å<=R<=3.2 Å, θ>90°, Φ−109.5°<90° for the sp³ donor—sp³ acceptor case, and, Φ>90° for the sp³ donor—sp² acceptor case; preferably, no switching functions are used. Template donors and acceptors that are involved in template-template hydrogen bonds are preferably not included in the donor and acceptor lists. For the purpose of exclusion, a template-template hydrogen bond is considered to exist when 2.5 Å<=R<=3.3 Å and θ>=135°.

The hydrogen-bond potential may also be combined or used with a weak Coulombic term that includes a distance-dependent dielectric constant of 40R, where R is the interatomic distance. Partial atomic charges are preferably only applied to polar functional groups. A net formal charge of +1 is used for Arg and Lys and a net formal charge of −1 is used for Asp and Glu; see Gasteiger, et al., Tetrahedron 36:3219-3288 (1980); Rappe, et al., J. Phys. Chem. 95:3358-3363 (1991).

In a preferred embodiment, an explicit penalty is given for buried polar hydrogen atoms which are not hydrogen bonded to another atom. See Eisenberg, et al., (1986) (supra), hereby expressly incorporated by reference. In a preferred embodiment, this penalty for polar hydrogen burial, is from about 0 to about 3 kcal/mol, with from about 1 to about 3 being preferred and 2 kcal/mol being particularly preferred. This penalty is only applied to buried polar hydrogens not involved in hydrogen bonds. A hydrogen bond is considered to exist when EHB ranges from about 1 to about 4 kcal/mol, with EHB of less than −2 kcal/mol being preferred. In addition, in a preferred embodiment, the penalty is not applied to template hydrogens, i.e. unpaired buried hydrogens of the backbone.

In a preferred embodiment, only hydrogen bonds between a first rotamer and the backbone are scored, and rotamer-rotamer hydrogen bonds are not scored. In an alternative embodiment, hydrogen bonds between a first rotamer and the backbone are scored, and rotamer-rotamer hydrogen bonds are scaled by 0.5.

In a preferred embodiment, the hydrogen bonding scoring function is used for all positions, including core, surface and boundary positions. In alternate embodiments, the hydrogen bonding scoring function may be used on only one or two of these.

In a preferred embodiment, a secondary structure propensity scoring function is used. This is based on the specific amino acid side-chain, and is conformation independent. That is, each amino acid has a certain propensity to take on a secondary structure, either a-helix or sheet, based on its Φ and ψ angles. See Munoz et al., Current Op. in Biotech. 6:382 (1995); Minor, et al., Nature 367:660-663 (1994); Padmanabhan, et al., Nature 344:268-270 (1990); Munoz, et al., Folding & Design 1(3):167-178 (1996); and Chakrabartty, et al., Protein Sci. 3:843 (1994), all of which are expressly incorporated herein by reference. Thus, for variable residue positions that are in recognizable secondary structure in the backbone, a secondary structure propensity scoring function is preferably used. That is, when a variable residue position is in an α-helical area of the backbone, the α-helical propensity scoring function described below is calculated. Whether or not a position is in an α-helical area of the backbone is determined as will be appreciated by those in the art, generally on the basis of Φ and ψ angles; for α-helix, Φ angles from −2 to −70 and ψ angles from −30 to −100 generally describe an α-helical area of the backbone.

Similarly, when a variable residue position is in a β-sheet backbone conformation, the β-sheet propensity scoring function is used. β-sheet backbone conformation is generally described by Φ angles from −30 to −100 and χ angles from +40 to +180. In alternate preferred embodiments, variable residue positions which are within areas of the backbone which are not assignable to either β-sheet or alpha.-helix structure may also be subjected to secondary structure propensity calculations.

In a preferred embodiment, energies associated with secondary propensities are calculated using Equation 14:

E=10^{Nss(ΔG°aa-ΔG°ala)}−1 Equation 14)

In Equation 14, Eα (or Eβ) is the energy of α-helical propensity, ΔG°ₐₐ is the standard free energy of helix propagation of the amino acid, and ΔG°ₐₗₐ is the standard free energy of helix propagation of alanine used as a standard, or standard free energy of β-sheet formation of the amino acid, both of which are available in the literature (see Chakrabartty, et al., (1994) (supra), and Munoz, et al., Folding & Design 1(3):167-178 (1996)), both of which are expressly incorporated herein by reference), and Nₛₛ is the propensity scale factor which is set to range from 1 to 4, with 3.0 being preferred. This potential is preferably selected in order to scale the propensity energies to a similar range as the other terms in the scoring function.

In a preferred embodiment, β-sheet propensities are preferably calculated only where the i−1 and i+1 residues are also in β-sheet conformation.

In a preferred embodiment, the secondary structure propensity scoring function is used only in the energy calculations for surface variable residue positions. In alternate embodiments, the secondary structure propensity scoring function is used in the calculations for core and boundary regions as well.

In a preferred embodiment, an electrostatic scoring function is used, as shown below in Equation 15:

E_{elec}=qq′/εr² (Equation 15)

In this Equation, q is the charge on atom 1, q′ is charge on atom 2, and r is the interaction distance.

In a preferred embodiment, at least one scoring function is used for each variable residue position; in preferred embodiments, two, three or four scoring functions are used for each variable residue position.

Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered.

In a preferred embodiment, the first step of the computational processing is done by calculating two sets of interactions for each rotamer at every position: the interaction of the rotamer side-chain with the template or backbone (the “singles” energy), and the interaction of the rotamer side-chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid or analog backbone.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the EHB is calculated for each possible rotamer at every variable position. Similarly, for the van der Waals scoring function, every atom of the rotamer is compared to every atom of the template (generally excluding the backbone atoms of its own residue), and the E_{vdw} is calculated for each possible rotamer at every variable residue position. In addition, generally no van der Waals energy is calculated if the atoms are connected by three bonds or less. For the atomic solvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

Accordingly, as outlined above, the total singles energy is the sum of the energy of each scoring function used at a particular position, as shown in Equation 1, wherein n is either 1 or zero, depending on whether that particular scoring function was used at the rotamer position:

Eₜₒₜₐₗ=nE_{vdw}+nEₐₛ+nE_{h-bonding}+nEₛₛ+nE_{elec} (Equation 1)

Once calculated, each singles Eₜₒₜₐₗ for each possible rotamer is stored, such that it may be used in subsequent calculations, as outlined below.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{EB} is calculated for each possible rotamer pair for any two variable positions. Similarly, for the van der Waals scoring function, every atom of the first rotamer is compared to every atom of every possible second rotamer, and the E_{vdW} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

Accordingly, as outlined above, the total doubles energy is the sum of the energy of each scoring function used to evaluate every possible pair of rotamers, as shown in Equation 16, wherein n is either 1 or zero, depending on whether that particular scoring function was used at the rotamer position:

Eₜₒₜₐₗ=nE_{vdw}+nEₐₛ+nE_{h-bonding}+E_{elec} (Equation 16)

An example is illuminating. A first variable position, i, has three (an unrealistically low number) possible rotamers (which may be either from a single amino acid or different amino acids) which are labeled ia, ib, and ic. A second variable position, j, also has three possible rotamers, labeled jd, je, and jf. Thus, nine doubles energies (Eₜₒₜₐₗ) are calculated in all: Wₜₒₜₐₗ (ia, jd), Eₜₒₜₐₗ (ia, je), Etₜₒₜₐₗ (ia, jf), Eₜₒₜₐₗ (ib, jd), Bₜₒₜₐₗ (ib, je), Eₜₒₜₐₗ (ib, if), Eₜₒₜₐₗ (ic, jd), Eₜₒₜₐₗ (ic, je), and Etₜₒₜₐₗ (ic, jf).

Once calculated, each doubles Eₜₒₜₐₗ for each possible rotamer pair is stored, such that it may be used in subsequent calculations, as outlined below.

Once the singles and doubles energies are calculated and stored, the next step of the computational processing may occur. Generally speaking, the goal of the computational processing is to determine a set of optimized protein sequences. By “optimized protein sequence” herein is meant a sequence that best fits the mathematical equations herein. As will be appreciated by those in the art, a global optimized sequence is the one sequence that best fits Equation 1, i.e. the sequence that has the lowest energy of any possible sequence. However, there are any number of sequences that are not the global minimum but that have low energies.

In a preferred embodiment, the set comprises the globally optimal sequence in its optimal conformation, i.e. the optimum rotamer at each variable position. That is, computational processing is run until the simulation program converges on a single sequence which is the global optimum.

In a preferred embodiment, the set comprises at least two optimized protein sequences. Thus for example, the computational processing step may eliminate a number of disfavored combinations but be stopped prior to convergence, providing a set of sequences of which the global optimum is one. In addition, further computational analysis, for example using a different method, may be run on the set, to further eliminate sequences or rank them differently. Alternatively, as is more fully described below, the global optimum may be reached, and then further computational processing may occur, which generates additional optimized sequences in the neighborhood of the global optimum.

If a set comprising more than one optimized protein sequences is generated, they may be rank ordered in terms of theoretical quantitative stability, as is more fully described below.

In a preferred embodiment, the computational processing step first comprises an elimination step, sometimes referred to as “applying a cutoff”, either a singles elimination or a doubles elimination. Singles elimination comprises the elimination of all rotamers with template interaction energies of greater than about 10 kcal/mol prior to any computation, with elimination energies of greater than about 15 kcal/mol being preferred and greater than about 25 kcal/mol being especially preferred. Similarly, doubles elimination is done when a rotamer has interaction energies greater than about 10 kcal/mol with all rotamers at a second residue position, with energies greater than about 15 being preferred and greater than about 25 kcal/mol being especially preferred.

In a preferred embodiment, the computational processing comprises direct determination of total sequence energies, followed by comparison of the total sequence energies to ascertain the global optimum and rank order the other possible sequences, if desired. The energy of a total sequence is shown below in Equation 17:

$${E}_{total protein} = {E}_{( b - b )} + {\sum }_{all\_i} {E}_{( ia )} + {\sum }_{all\_i , j\_pairs} \sum {E}_{( ia , ja )} \qquad ( Equation 17 )$$

Thus every possible combination of rotamers may be directly evaluated by adding the backbone-backbone (sometimes referred to herein as template-template) energy E_{b-b}) which is constant over all sequences herein since the backbone is kept constant), the singles energy for each rotamer (which has already been calculated and stored), and the doubles energy for each rotamer pair (which has already been calculated and stored). Each total sequence energy of each possible rotamer sequence can then be ranked, either from best to worst or worst to best. This is obviously computationally expensive and becomes unwieldy as the length of the protein increases.

In a preferred embodiment, the computational processing includes one or more Dead-End Elimination (DEE) computational steps. The DEE theorem is the basis for a very fast discrete search program that was designed to pack protein side-chains on a fixed backbone with a known sequence. See Desmet, et al., Nature 356:539-542 (1992); Desmet, et al., The Proteins Folding Problem and Tertiary Structure Prediction, Ch. 10:1-49 (1994); Goldstein, Biophys. Jour. 66:1335-1340 (1994), all of which are incorporated herein by reference. DEE is based on the observation that if a rotamer can be eliminated from consideration at a particular position, i.e. make a determination that a particular rotamer is definitely not part of the global optimal conformation, the size of the search is reduced. This is done by comparing the worst interaction (i.e. energy or Eₜₒₜₐₗ) of a first rotamer at a single variable position with the best interaction of a second rotamer at the same variable position. If the worst interaction of the first rotamer is still better than the best interaction of the second rotamer, then the second rotamer cannot possibly be in the optimal conformation of the sequence. The original DEE theorem is shown in Equation 18:

E(ia)+Σ[min(j) over t{E(ia, jt)}]>E(ib)+[max(j) over t{E(ib, jt)}] (Equation 18)

In Equation 18, rotamer ia is being compared to rotamer ib. The left side of the inequality is the best possible interaction energy (Eₜₒₜₐₗ) of ia with the rest of the protein; that is, “min over t” means find the rotamer t on position j that has the best interaction with rotamer ia. Similarly, the right side of the inequality is the worst possible (max) interaction energy of rotamer ib with the rest of the protein. If this inequality is true, then rotamer ia is Dead-Ending and can be Eliminated. The speed of DEE comes from the fact that the theorem only requires sums over the sequence length to test and eliminate rotamers.

In a preferred embodiment, a variation of DEE is performed. Goldstein DEE, based on Goldstein, (1994) (supra), hereby expressly incorporated by reference, is a variation of the DEE computation, as shown in Equation 19:

E(ia)−E(ib)+Σ[min over t{E(ia,jt)−E(ib,jt)}]>0 (Equation 19)

In essence, the Goldstein Equation 19 says that a first rotamer a of a particular position i (rotamer ia) will not contribute to a local energy minimum if the energy of conformation with ia can always be lowered by just changing the rotamer at that position to ib, keeping the other residues equal. If this inequality is true, then rotamer ia is Dead-Ending and can be Eliminated.

Thus, in a preferred embodiment, a first DEE computation is done where rotamers at a single variable position are compared, (“singles” DEE) to eliminate rotamers at a single position. This analysis is repeated for every variable position, to eliminate as many single rotamers as possible. In addition, every time a rotamer is eliminated from consideration through DEE, the minimum and maximum calculations of Equation 18 or 19 change, depending on which DEE variation is used, thus conceivably allowing the elimination of further rotamers. Accordingly, the singles DEE computation can be repeated until no more rotamers can be eliminated; that is, when the inequality is not longer true such that all of them could conceivably be found on the global optimum.

In a preferred embodiment, “doubles” DEE is additionally done. In doubles DEE, pairs of rotamers are evaluated; that is, a first rotamer at a first position and a second rotamer at a second position are compared to a third rotamer at the first position and a fourth rotamer at the second position, either using original or Goldstein DEE. Pairs are then flagged as nonallowable, although single rotamers cannot be eliminated, only the pair. Again, as for singles DEE, every time a rotamer pair is flagged as nonallowable, the minimum calculations of Equation 18 or 19 change (depending on which DEE variation is used) thus conceivably allowing the flagging of further rotamer pairs. Accordingly, the doubles DEE computation can be repeated until no more rotamer pairs can be flagged; that is, where the energy of rotamer pairs overlap such that all of them could conceivably be found on the global optimum.

In addition, in a preferred embodiment, rotamer pairs are initially prescreened to eliminate rotamer pairs prior to DEE. This is done by doing relatively computationally inexpensive calculations to eliminate certain pairs up front. This may be done in several ways, as is outlined below.

In a preferred embodiment, the rotamer pair with the lowest interaction energy with the rest of the system is found. Inspection of the energy distributions in sample matrices has revealed that an iᵤjᵥ pair that dead-end eliminates a particular iᵣjₛ pair can also eliminate other iᵣjₛ pairs. In fact, there are often a few iᵤjᵥ pairs, which we call “magic bullets,” that eliminate a significant number of iᵣjₛ pairs. We have found that one of the most potent magic bullets is the pair for which maximum interaction energy, tₘₐₓ([iᵤjᵥ])kₜ, is least. This pair is referred to as [iᵤjᵥ]mb. If this rotamer pair is used in the first round of doubles DEE, it tends to eliminate pairs faster.

Our first speed enhancement is to evaluate the first-order doubles calculation for only the matrix elements in the row corresponding to the [iᵤ iᵥ]mb pair. The discovery of [iᵤjᵥ]_{mb} is an n² calculation (n=the number of rotamers per position), and the application of Equation 19 to the single row of the matrix corresponding to this rotamer pair is another n² calculation, so the calculation time is small in comparison to a full first-order doubles calculation. In practice, this calculation produces a large number of dead-ending pairs, often enough to proceed to the next iteration of singles elimination without any further searching of the doubles matrix.

The magic bullet first-order calculation will also discover all dead-ending pairs that would be discovered by the Equation 18 or 19, thereby making it unnecessary. This stems from the fact that .epsilon.ₘₐₓ ([iᵤ ijᵥ]mb) must be less than or equal to any .epsilon.ₘₐₓ ([iᵤjᵥ]) that would successfully eliminate a pair by the Equation 18 or 19.

Since the minima and maxima of any given pair has been precalculated as outlined herein, a second speed-enhancement precalculation may be done. By comparing extrema, pairs that will not dead end can be identified and thus skipped, reducing the time of the DEE calculation. Thus, pairs that satisfy either one of the following criteria are skipped:

εₘᵢₙ([iᵣjₛ])<εₘᵢₙ([iᵤjᵥ]) (Equation 20)

εₘᵢₙ[iᵣjₛ])<εₘᵢₙ([iᵤjᵥ]) (Equation 21)

Because the matrix containing these calculations is symmetrical, half of its elements will satisfy the first inequality Equation 20, and half of those remaining will satisfy the other inequality Equation 21. These three quarters of the matrix need not be subjected to the evaluation of Equation 18 or 19, resulting in a theoretical speed enhancement of a factor of four.

The last DEE speed enhancement refines the search of the remaining quarter of the matrix. This is done by constructing a metric from the precomputed extrema to detect those matrix elements likely to result in a dead-ending pair.

A metric was found through analysis of matrices from different sample optimizations. We searched for combinations of the extrema that predicted the likelihood that a matrix element would produce a dead-ending pair. Interval sizes for each pair were computed from differences of the extrema. The size of the overlap of the iᵣ jₛ and iᵤ jᵥ intervals were also computed, as well as the difference between the minima and the difference between the maxima. Combinations of these quantities, as well as the lone extrema, were tested for their ability to predict the occurrence of dead-ending pairs. Because some of the maxima were very large, the quantities were also compared logarithmically.

Most of the combinations were able to predict dead-ending matrix elements to varying degrees. The best metrics were the fractional interval overlap with respect to each pair, referred to herein as qᵣₛ and qᵤᵥ.

$$\begin{matrix}\begin{matrix}{q}_{rs} = \text{interval overlap/interval} ( [ {i}_{r} {j}_{s} ] ) \\ = \{ {ϵ}_{max} ( [ {i}_{u} {j}_{v} ] ) - {ϵ}_{min} ( [ {i}_{r} {j}_{s} ] ) \} / \\ \{ {ϵ}_{max} ( [ {i}_{r} {j}_{s} ] ) - {ϵ}_{min} ( [ {i}_{r} {j}_{s} ] ) \}\end{matrix} & ( Equation 22 ) \\ \begin{matrix}{q}_{uv} = \text{interval overlap/interval} ( [ {i}_{u} {j}_{v} ] ) \\ = \{ {ϵ}_{max} ( [ {i}_{u} {j}_{v} ] ) - {ϵ}_{min} ( [ {i}_{r} {j}_{s} ] ) \} / \\ \{ {ϵ}_{max} ( [ {i}_{u} {j}_{v} ] ) - {ϵ}_{min} ( [ {i}_{u} {j}_{v} ] ) \}\end{matrix} & ( Equation 23 )\end{matrix}$$

These values are calculated using the minima and maxima equations 24, 25, 26 and 27:

$$\begin{matrix}{ϵ}_{max} ( [ {i}_{r} {j}_{s} ] ) = ϵ ( [ {i}_{r} {j}_{s} ] ) + {\sum }_{k \neq i \neq j} max ( l ) ϵ ( [ {i}_{r} {j}_{s} ] , k ) & ( Equation 24 ) \\ {ϵ}_{min} ( [ {i}_{r} {j}_{s} ] ) = ϵ ( [ {i}_{r} {j}_{s} ] ) + {\sum }_{k \neq i \neq j} min ( t ) ϵ ( [ {i}_{r} {j}_{s} ] , {k}_{l} ) & ( Equation 25 ) \\ {ϵ}_{max} ( [ {i}_{u} {j}_{v} ] ) = ϵ ( [ {i}_{u} {j}_{v} ] ) + {\sum }_{k \neq i \neq j} max ( t ) ϵ ( [ {i}_{u} {j}_{v} ] , {k}_{t} ) & ( Equation 26 ) \\ {ϵ}_{min} ( [ {i}_{u} {j}_{v} ] ) = ϵ ( [ {i}_{u} {j}_{v} ] ) + {\sum }_{k \neq i \neq j} min ( t ) ϵ ( [ {i}_{u} {j}_{v} ] , {k}_{l} ) & ( Equation 27 )\end{matrix}$$

These metrics were selected because they yield ratios of the occurrence of dead-ending matrix elements to the total occurrence of elements that are higher than any of the other metrics tested. For example, there are very few matrix elements (˜2%) for which qᵣₛ>0.98, yet these elements produce 30-40% of all of the dead-ending pairs.

Accordingly, the first-order doubles criterion is applied only to those doubles for which qᵣₛ>0.98 and qᵤᵥ>0.99. The sample data analyses predict that by using these two metrics, as many as half of the dead-ending elements may be found by evaluating only two to five percent of the reduced matrix.

Generally, as is more fully described below, single and double DEE, using either or both of original DEE and Goldstein DEE, is run until no further elimination is possible. Usually, convergence is not complete, and further elimination must occur to achieve convergence. This is generally done using “super residue” DEE.

In a preferred embodiment, additional DEE computation is done by the creation of “super residues” or “unification”, as is generally described in Desmet, Nature 356:539-542 (1992); Desmet, et al., The Protein Folding Problem and Tertiary Structure Prediction, Ch. 10:1-49 (1994); Goldstein, et al., supra. A super residue is a combination of two or more variable residue positions which is then treated as a single residue position. The super residue is then evaluated in singles DEE, and doubles DEE, with either other residue positions or super residues. The disadvantage of super residues is that there are many more rotameric states which must be evaluated; that is, if a first variable residue position has 5 possible rotamers, and a second variable residue position has 4 possible rotamers, there are 20 possible super residue rotamers which must be evaluated. However, these super residues may be eliminated similar to singles, rather than being flagged like pairs.

The selection of which positions to combine into super residues may be done in a variety of ways. In general, random selection of positions for super residues results in inefficient elimination, but it can be done, although this is not preferred. In a preferred embodiment, the first evaluation is the selection of positions for a super residue is the number of rotamers at the position. If the position has too many rotamers, it is never unified into a super residue, as the computation becomes too unwieldy. Thus, only positions with fewer than about 100,000 rotamers are chosen, with less than about 50,000 being preferred and less than about 10,000 being especially preferred.

In a preferred embodiment, the evaluation of whether to form a super residue is done as follows. All possible rotamer pairs are ranked using Equation 28, and the rotamer pair with the highest number is chosen for unification:

Fraction of flagged pairs/log^{(number of super rotamers resulting from the potential unification)} (Equation 28)

Equation 28 is looking for the pair of positions that has the highest fraction or percentage of flagged pairs but the fewest number of super rotamers. That is, the pair that gives the highest value for Equation 28 is preferably chosen. Thus, if the pair of positions that has the highest number of flagged pairs but also a very large number of super rotamers (that is, the number of rotamers at position i times the number of rotamers at position j), this pair may not be chosen (although it could) over a lower percentage of flagged pairs but fewer super rotamers.

In an alternate preferred embodiment, positions are chosen for super residues that have the highest average energy; that is, for positions i and j, the average energy of all rotamers for i and all rotamers for j is calculated, and the pair with the highest average energy is chosen as a super residue.

Super residues are made one at a time, preferably. After a super residue is chosen, the singles and doubles DEE computations are repeated where the super residue is treated as if it were a regular residue. As for singles and doubles DEE, the elimination of rotamers in the super residue DEE will alter the minimum energy calculations of DEE. Thus, repeating singles and/or doubles DEE can result in further elimination of rotamers.

In summary, the calculation and storage of the singles and doubles energies is the first step, although these may be recalculated every time. This is followed by the optional application of a cutoff, where singles or doubles energies that are too high are eliminated prior to further processing. Either or both of original singles DEE or Goldstein singles DEE may be done, with the elimination of original singles DEE being generally preferred. Once the singles DEE is run, original doubles and/or Goldstein doubles DEE is run. Super residue DEE is then generally run, either original or Goldstein super residue DEE. This preferably results in convergence at a global optimum sequence. After any step any or all of the previous steps can be rerun, in any order.

The addition of super residue DEE to the computational processing, with repetition of the previous DEE steps, generally results in convergence at the global optimum. Convergence to the global optimum is guaranteed if no cutoff applications are made, although generally a global optimum is achieved even with these steps. In a preferred embodiment, DEE is run until the global optimum sequence is found. That is, the set of optimized protein sequences contains a single member, the global optimum.

In a preferred embodiment, the various DEE steps are run until a manageable number of sequences is found, i.e. no further processing is required. These sequences represent a set of optimized protein sequences, and they can be evaluated as is more fully described below. Generally, for computational purposes, a manageable number of sequences depends on the length of the sequence, but generally ranges from about 1 to about 1015 possible rotamer sequences.

Alternatively, DEE is run to a point, resulting in a set of optimized sequences (in this context, a set of remainder sequences) and then further computational processing of a different type may be run. For example, in one embodiment, direct calculation of sequence energy as outlined above is done on the remainder possible sequences. Alternatively, a Monte Carlo search can be run.

In a preferred embodiment, the computation processing need not comprise a DEE computational step. In this embodiment, a Monte Carlo search is undertaken, as is known in the art. See Metropolis et al., J. Chem. Phys. 21:1087 (1953), hereby incorporated by reference. In this embodiment, a random sequence comprising random rotamers is chosen as a start point. In one embodiment, the variable residue positions are classified as core, boundary or surface residues and the set of available residues at each position is thus defined. Then a random sequence is generated, and a random rotamer for each amino acid is chosen. This serves as the starting sequence of the Monte Carlo search. A Monte Carlo search then makes a random jump at one position, either to a different rotamer of the same amino acid or a rotamer of a different amino acid, and then a new sequence energy (Eₜₒₜₐₗ sequence) is calculated, and if the new sequence energy meets the Boltzmann criteria for acceptance, it is used as the starting point for another jump. If the Boltzmann test fails, another random jump is attempted from the previous sequence. In this way, sequences with lower and lower energies are found, to generate a set of low energy sequences.

If computational processing results in a single global optimum sequence, it is frequently preferred to generate additional sequences in the energy neighborhood of the global solution, which may be ranked. These additional sequences are also optimized protein sequences. The generation of additional optimized sequences is generally preferred so as to evaluate the differences between the theoretical and actual energies of a sequence. Generally, in a preferred embodiment, the set of sequences is at least about 75% homologous to each other, with at least about 80% homologous being preferred, at least about 85% homologous being particularly preferred, and at least about 90% being especially preferred. In some cases, homology as high as 95% to 98% is desirable. Homology in this context means sequence similarity or identity, with identity being preferred. Identical in this context means identical amino acids at corresponding positions in the two sequences which are being compared. Homology in this context includes amino acids which are identical and those which are similar (functionally equivalent). This homology will be determined using standard techniques known in the art, such as the Best Fit sequence program described by Devereux, et al., Nucl. Acid Res., 12:387-395 (1984), or the BLASTX program (Altschul, et al., J. Mol. Biol., 215:403-410 (1990)) preferably using the default settings for either. The alignment may include the introduction of gaps in the sequences to be aligned. In addition, for sequences which contain either more or fewer amino acids than an optimum sequence, it is understood that the percentage of homology will be determined based on the number of homologous amino acids in relation to the total number of amino acids. Thus, for example, homology of sequences shorter than an optimum will be determined using the number of amino acids in the shorter sequence.

Once optimized protein sequences are identified, the processing optionally proceeds to a step which entails searching the protein sequences. This processing may be implemented with a set of computer code that executes a search strategy. For example, the search may include a Monte Carlo search as described above. Starting with the global solution, random positions are changed to other rotamers allowed at the particular position, both rotamers from the same amino acid and rotamers from different amino acids. A new sequence energy (Eₜₒₜₐₗ sequence) is calculated, and if the new sequence energy meets the Boltzmann criteria for acceptance, it is used as the starting point for another jump. See Metropolis et al., 1953, supra, hereby incorporated by reference. If the Boltzmann test fails, another random jump is attempted from the previous sequence. A list of the sequences and their energies is maintained during the search. After a predetermined number of jumps, the best scoring sequences may be output as a rank-ordered list. Preferably, at least about 10⁶ jumps are made, with at least about 10⁷ jumps being preferred and at least about 10⁸ jumps being particularly preferred. Preferably, at least about 100 to 1000 sequences are saved, with at least about 10,000 sequences being preferred and at least about 100,000 to 1,000,000 sequences being especially preferred. During the search, the temperature is preferably set to 1000 K.

Once the Monte Carlo search is over, all of the saved sequences are quenched by changing the temperature to 0 K, and fixing the amino acid identity at each position. Preferably, every possible rotamer jump for that particular amino acid at every position is then tried.

The computational processing results in a set of optimized protein sequences that are best suited to bind the intended amino acid analog. These optimized protein sequences may be significantly different from the wild-type sequence from which the backbone was taken. That is, each optimized protein sequence may comprises at least one residue change, or at least about 1-2%, 2-5%, 5-10% or more variant amino acids from the starting or wild-type sequence.

In a preferred embodiment, one, some or all of the optimized redesigned protein sequences are constructed into designed proteins. Thereafter, the optimized redesigned protein sequences can be tested for their ability, specificity, efficiency or any other biological activity in in vitro and/or in vivo assays. Generally, this can be done in one of two ways.

The mutated amino-acid sequences obtained from the ORBIT algorithms are subsequently generated in the laboratory (either by peptide synthesis or total gene synthesis via recursive PCR) and their binding properties assessed with conventional biophysical techniques. For example, various biochemical methods and techniques can be used to purify the expressed proteins (e.g. FPLC and HPLC) and further assess the degree of complex formation either in vitro (e.g. size-exclusion chromatography, analytical ultracentrifugation, etc.), or in vivo (yeast two-hybrid test, immunoprecipitation, or any other functional assays, etc.), or both. Finally, to verify that the designed proteins are docked in the target orientation, the structure of each complex can be solved by either multidimensional NMR or x-ray crystallography.

In a preferred embodiment, the experimental results are used for design feedback and design optimization. This cyclic approach ultimately increases the understanding of the forces that drive intermolecular interaction, and raises the likelihood of successful protein-protein complex design.

In addition, the order in which the steps of the present method are performed is purely illustrative in nature. In fact, the steps can be performed in any order or in parallel, unless otherwise indicated by the present disclosure.

Furthermore, the method of the present invention may be performed in either hardware, software, or any combination thereof, as those terms are currently known in the art. In particular, the present method may be carried out by software, firmware, or microcode operating on a computer or computers of any type. Additionally, software embodying the present invention may comprise computer instructions in any form (e.g., source code, object code, interpreted code, etc.) stored in any computer-readable medium (e.g., ROM, RAM, magnetic media, punched tape or card, compact disc (CD) in any form, DVD, etc.). Furthermore, such software may also be in the form of a computer data signal embodied in a carrier wave, such as that found within the well-known Web pages transferred among devices connected to the Internet. Accordingly, the present invention is not limited to any particular platform, unless specifically stated otherwise in the present disclosure.

Exemplery computer hardware means suitable for carrying out the invention can be a Silicon Graphics Power Challenge server with 10 R10000 processors running in parallel. Suitable software development environment includes CERIUS2 by Biosym/Molecular Simulations (San Diego, Calif.), or other equivalents.

While particular embodiments of the present invention have been shown and described, it will be apparent to those skilled in the art that changes and modifications may be made without departing from this invention in its broader aspect and, therefore, the appended claims are to encompass within their scope all such changes and modifications as fall within the true spirit of this invention.

IV. Exemplary Uses

In theory, the instant invention can be used in any situations where interaction between two or more molecules, especially those involving at least one protein molecule, need to be rationally designed. The following uses are just a few illustrative examples, and are by no means limiting. A skilled artisan can readily envision other potential uses of the invention.

In one embodiment, the instant invention can be used to design one of the two interacting molecules. For example, a candidate molecule may be redesigned based on a target molecule. More specifically, if the structure of a target protein is known, the structure of a candidate protein may be redesigned so that it binds the target with better specificity and/or affinity.

To illustrate, the instant invention can be used to redesign antibodies or functional fragment thereof, so that they bind selected epitopes with more specificity and/or avidity. The term antibody as used herein is intended to include functional fragments thereof which retains substantially the same binding property of the native antibody (monoclonal or polyclonal). Antibodies can be fragmented using conventional techniques and the fragments screened for utility in the same manner as described for whole antibodies. For example, F(ab)₂ fragments can be generated by treating antibody with pepsin. The resulting F(ab)₂ fragment can be treated to reduce disulfide bridges to produce Fab fragments. An antibody of the present invention is further intended to include bispecific, single-chain, and chimeric and humanized molecules conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. In certain embodiments, the antibodies further comprises a label attached thereto and able to be detected (e.g., the label can be a radioisotope, fluorescent compound, enzyme or enzyme co-factor).

The general 3D structures of the immunoglobulins are well-known in the art, the redesign can thus be focused on the CDR sequences of the H and/or L chains. This can be used to redesign antibodies that more selectively bind one antigen, as compared to a closely related antigen. For example, the HER2/neu oncogene is a mutated form of the c-erbB2 receptor found in many metastatic breast cancer cells. A humanized monoclonal antibody, HERCEPTIN™ (Genentech), is the first monoclonal antibody to be approved by the Food and Drug Administration (FDA) for the treatment of advanced metastatic breast cancer. The antibody specifically binds the HER2 receptor, which contains a single point mutation when compared to the wild type c-erbB2 receptor, leading to the eventual killing of cancer cells overexpressing this mutant receptor. Unfortunately, there are at least two severe side-effects of using HERCEPTIN in human patients, including cardiomyopathy and various forms of hypersensitivity reactions. Thus, it is conceivable that the instant invention can be used to increased the selectivity of HERCEPTIN for the HER2 receptor, while decreasing the effective dose due to its higher avidity/selectivity, therefore potentially lowering such undesirable side effects.

A similar method may be useful for designing novel CDR sequences of a scaffold immunoglobulin molecule (or a functional fragment thereof) for recognition of a given epitope. For example, if there is a need to use antibody to block a specific function of a target molecule by binding to a particular epitope on the target molecule, the instant invention can be used design CDR sequences that best fit the contour of the target epitope, followed by side-chain selection to identify the best CDR sequence for binding to said epitope.

In another example, protein transcription factors binds specific (short) DNA sequence and modulates transcription. It might be desirable to change the nucleotide recognition specificity and/or affinity of a particular transcription factor, thus conferring the redesigned transcription factor with novel activity (recognize different DNA sequences, binds DNA with modified affinity, etc.) Since the 3D structure of many transcription factors in complex with their respective DNA recognition sequences are known, the instant invention may be used to selectively redesign the interface side-chains of the transcription factor in contact with the nucleotides of the DNA.

Similarly, protein binding other non-polypeptide molecules, such as lipids (PI, etc.), sugar moieties, steroids, metal atoms, vitamin cofactors, etc. may also be redesigned based on specific needs, such as change enzyme specificity/activity.

In an alternative embodiment, a protein target may be fixed as the target molecule, while a non-protein candidate molecule (including a peptide mimetic with modified backbones and/or side-chains) may be may be redesigned by changing atoms in contact with the target protein.

In another embodiment, the instant invention can be used to design small molecules (such as small peptides) that selectively disrupt the binding between two molecules. For example, if two proteins are known to bind each others one of the proteins may be chosen as the target molecule, and the binding interface of the other molecule (the candidate molecule) can be redesigned to enhance the binding (higher binding affinity, etc.). Based on the sequence of the redesigned interface, a peptide fragment representing the binding interface sequence of the candidate molecule may be obtained. Since the redesigned binding interface is expected to have a higher binding affinity for the target molecule, the peptide fragment is expected to be able to better disrupt the candidate-target complex.

In another embodiment, the instant invention may be used to design/identify a small molecule (for example, a molecule smaller than 5 k/a) that enhances the binding between two macromolecules. Foe example, there may be “gaps” between the binding interface of two interacting proteins. A small molecule capable of fitting into the gap may form multiple interactions with both macromolecules, thus strengthening the overall complex stability. For that purpose, the two macromolecules in complex may be treated as a single large molecule, while at least one candidate small molecules may be tested for best fit in the “gap,” and then the atoms of such small molecules in contact with the macromolecules can be redesigned to find a best fit.

In another embodiment, the instant invention can be used to redesign, mutate and drive small proteins to self-assemble into complexes of specific structure (e.g. precise dimer formation). The small proteins can then be redesigned to bind to specific regions of target proteins expressed by pathogenic organisms. These design targets can be geared towards applications in the field of protein-based drug design.

### EXAMPLES

The examples below are for the purpose of illustration only, and should not be construed to be limiting in any respect.

### Example 1

### Redesigning Monomeric Protein (Protein G) for Self-Assembly

The general aim of this experiment is to combine the principles of supramolecular chemistry with the emerging tools of protein engineering. The goal is to increase our understanding of the underlying physical principles of molecular self-assembly and thus enable us to design the building blocks and raw material for the emerging field of biological material science. The initial engineering goal is to redesign monomeric proteins such that they self-assemble into complexes of predefined specific structure.

The first step in driving de novo self-assembly is the computational docking of the proteins together in the predefined orientation. To this end, the Applicants have modified an established docking algorithm, the Geometric Recognition Algorithm (GRA). The GRA treats the molecules as rigid bodies and rigorously assesses interfacial surface complementarity as a function of translational and rotational position. This process is computationally intensive yet has been rendered tractable by utilizing the Fourier Correlation Theorem.

Upon obtaining the optimal intermolecular atomic coordinates the two molecules are treated as one and a suite of highly developed protein design algorithms, which utilize advanced molecular mechanics force fields, is used to computationally repack the interfacial side-chains in a manner analogous to the cores of well folded proteins.

Applicants have successfully developed the above techniques and have preliminary results on driving a small protein (previously monomeric) to dimerize. Tools of molecular biology were used to introduce the mutations and produce the redesigned docked proteins. The extent and specificity of binding were assessed with analytical ultracentrifugation and heteronuclear NMR.

Protein Engineering and Supramolecular Biochemistry:

Molecular self-assembly is the spontaneous association of molecules into stable, structurally well-defined complexes joined by noncovalent bonds. Understanding self-assembly and the noncovalent interactions that connect interacting molecular surfaces is a main focus of supramolecular chemistry (Huc and Lehn, 1997). Unlike the traditional use of small organic molecules as building blocks of supramolecular structures, our methods mimic nature in that the designed building blocks are protein-based. The strength of this approach is that it relies on and exploits the large body of structural and biophysical data thus far compiled on biological macromolecules. Additionally it enables the use of powerfill in vivo genetic screens (e.g., bacterial two-hybrid screen) that sample large combinatorial libraries (i.e., 1×10⁹) for successful docking candidates.

Programmed Self-Assembly: De Novo Docking:

All organisms depend on the precise self-assembly of native proteins into functional multi-subunit complexes. The formation of these complexes is driven by the same forces that drive protein folding. Of particular importance is the hydrophobic effect. The propensity of proteins to sequester hydrophobic residues within their core is similar to that observed at the interfaces of obligate dimers (Jones and Thornton, 1997). Other important intermolecular interactions include hydrophilic effects, electrostatic interactions, hydrogen bonding and van der Waals interactions. The goal is to understand and ultimately control these forces. The approach taken is analogous to that taken for the inverse protein folding approach; instead of predicting how native complexes form, Applicants are determining which forces are essential by driving the de novo self-assembly of previously monomeric proteins.

The initial engineering goal is to redesign, mutate and drive proteins to self-assemble in a pre-defined, structurally specific fashion (e.g., precise dimer formation). The experimental approach entails a protein design cycle which combines Physical Chemistry (theory), Computer Science (simulation), Molecular Biology (recombinant DNA technologies), Biochemistry (protein purification) and Biophysical Analysis (spectroscopy).

De Novo Docking:

The de novo docking strategy used herein is summarized in the following five steps:

(1) Choose a small, well behaved, monomeric protein and general target orientation for the docked complex (i.e., the dimer structure).

(2) Computationally dock the backbones of the proteins in the target orientation and systematically ascertain the atomic coordinates which result in maximal subunit-to-subunit surface complementarity.

(3) Treat the two molecules as one and use established protein design algorithms to repack the side-chains at the protein-protein interface in a manner similar to that observed in the cores of well folded proteins. The protein design algorithms are contained in the ORBIT (Optimal Rotomers Based on Iterative Techniques) suite of algorithms (Dahiyat et al., 1997).

(4) Utilize standard tools of molecular biology and biochemistry to physically generate the redesigned monomers (e.g., total gene synthesis, recombinant DNA technology, recursive PCR, HPLC, FPLC, etc.).

(5) Assess the success of the complex formation with standard biophysical techniques (e.g., gel filtration, ultra centrifugation, NMR, x-ray crystallography).

The Monomeric Protein and Target Orientation (Step 1)

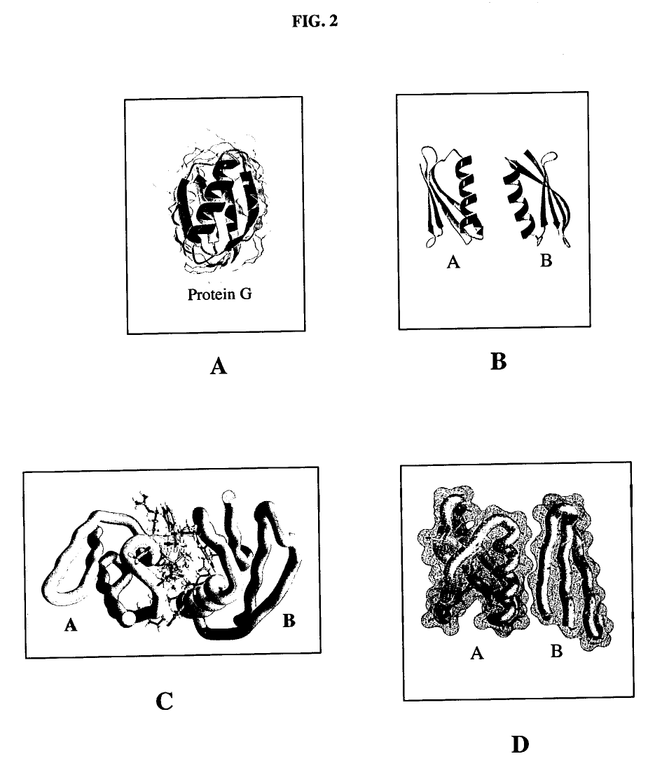

The β1 domain of the Streptococcal protein G (Gβ1, FIG. 2a) is a 56 amino acid domain which has been extensively redesigned and biophysically analyzed (Malakauskas and Mayo, 1998). This protein was chosen because it expresses well in E. Coli, is monomeric and well behaved in solution and its small compact structure has been determined to high resolution (Gronenborn et al., 1991; Gallagher et al., 1994). We chose as the initial target orientation a dual 180° rotation about the y and z axis's resulting in one molecule flipped head-to-tail and oriented helix-face to helix-face as shown in FIG. 2b.

Computational Docking and Maximizing Surface Complementarity (Step 2)

Although this target orientation is dictated, there is still a need to search the interfacial space to find the optimal surface-to-surface geometric fit. To accomplish this, a well established algorithm was borrowed from the field of native protein docking; the Geometric Recognition Algorithm (GRA) (Katchalski-Katzir et al., 1992; Gabb et al. 1997).

The GRA treats the two molecules as rigid bodies and uses surface complementarity as the criteria for goodness of fit. It does so by projecting the molecules onto a three-dimensional grid of N×N×N points where they are represented by the following discrete functions—

$$molecule {a}_{l , m , n} = \{ \begin{matrix} 1 surface of molecule \\ - 15 inside the molecule \\ 0 outside the molecule\end{matrix} molecule {b}_{l , m , n} = \{ \begin{matrix} 1 surface \text{/} inside of molecule \\ 0 outside the molecule \\ \end{matrix}$$

Matching of complementary surfaces is then accomplished by computing the following correlation function (Katchalski-Katzir et al., 1992; Gabb et al. 1997):

$$Correlation Function \text{:} {c}_{a , β , γ} = {\sum }_{n = 1}^{N} {\sum }_{m = 1}^{N} {\sum }_{l = 1}^{N} {a}_{l , m , n} \cdot {b}_{l + a , m + β , n + γ}$$

Although Applicants are dictating the relative orientation, a high resolution calculation of the correlation function remains computationally intensive. Therefore Applicants chose to utilize the Fourier correlation theorem (FCT) originally described by Katchalski-Katzir et al., 1992. The FCT reduces the number of translational calculations (i.e., ˜N⁶) down to the order of N³ In (N³) (Press et al., 1986 and Bracewell, 1990). Thus, we are able to reduce the computational complexity while maintaining a high degree of translational resolution.

Results

We have successfully developed the code for the de novo docking algorithm. Implementation of the FCT has rendered the computationally intense, high resolution grid search tractable. A single translational calculation, where N=128, which did not utilize the FCT, took ˜25.4 days to complete on a single SGI R10000 processor. Using the FCT the identical calculation was reduced to ˜58 s, a reduction of over 10,000 fold. Currently, on a Pentium IV, 1 GHz processor running Linux, the calculation takes ˜11.5 s.

High resolution scans (i.e., 0.5 Å; 5°/scan) of 5,832 different rotational backbone positions completed in a reasonable time frame (˜40 hr). To prevent structural bias of the interface the side-chain atoms of the wild-type residues were removed (except Cβ atoms). The orientation which exhibited the highest surface complementarity is shown in FIG. 2c (for clarity in illustrating the considerable interdigitation only the beta-sheet surface of monomer B is shown). The coordinates of this docked complex were used in the next step of the computational docking process.

Interfacial Side-Chain Selection Via the ORBIT Suite of Design Algorithms (Step 3)

An integral step in the docking process entails the ORBIT suite of protein design algorithms (Dahiyat et al., 1997). The algorithms are used to perform side-chain selection on interfacial residue positions. The primary function of these algorithms is to return a mutated protein sequence optimized for a given three-dimensional backbone structure (Street and Mayo, 1999). They employ an unbiased, quantitative design method based on the physical chemical properties that determine protein structure and stability (Gordon et al., 1999).

The RESLASS algorithm (which classifies a residue as core, boundary or surface based on its position in the molecule) was used to determine which residues become buried upon docking. 15 residues were reclassified as core and 7 as boundary. ORBIT was used to assess the energy of and select hydrophobic side-chains for the 15 interfacial core positions and hydrophilic side-chains for the 7 reclassified boundary positions. Due to favorable interfacial proximity 2 additional surface positions were included in the calculation.

FIG. 2d displays the side-chains of the 24 calculated positions. The total redesign resulted in a 20-fold mutant (12 for monomer A and 8 for B; 4 remained wild-type). Upon complex formation these mutant monomers bury ˜1560 Å² of surface area (˜76% of which is hydrophobic).

Construction of the Mutants and Protein Purification (Step 4)

Synthetic DNA oligos were used with recursive PCR for the total gene synthesis of the above two monomers. The genes were cloned into pET-11a (Novagen) and recombinant protein was expressed by IPTG induction in BL21(DE3) hosts (Invitrogen) and isolated using a freeze/thaw method. Purification was accomplished by reverse-phase HPLC using a linear 1% min−1 acetonitrile/water gradient containing 0.1% TFA. Molecular weights were verified by mass spectrometry.

Analytical Ultracentrifugation (Step 5)

Sedimentation equilibrium experiments were conducted in a Beckman XL-I Ultima analytical ultracentrifuge equipped with absorbance optics. Runs were carried out at 28,000, 40,000 and 48,000 rpm, at 20° C. Global nonlinear least-squares analysis of the data from the lowest speed in the initial run was consistent with weak dimerization, with a putative K_{d} of ˜300 μM. Although there are difficulties in the analysis of this data, as monomer B alone showed evidence of nonideality, the initial results indicate dimerization may be occurring, in agreement with preliminary 1D NMR analysis. To conduct further tests, multidimensional heteronuclear NMR was used to analyze the complex.

Heteronuclear NMR Analysis (Step 5)

NMR data were collected at 20° C. on a Varian UnityPlus 600 MHz spectrometer equipped with an HCN-triple-resonance probe with triple-axis pulse field gradients. Protein concentrations were ˜2.5 mM in 25 mM sodium phosphate, pH ˜6.5.

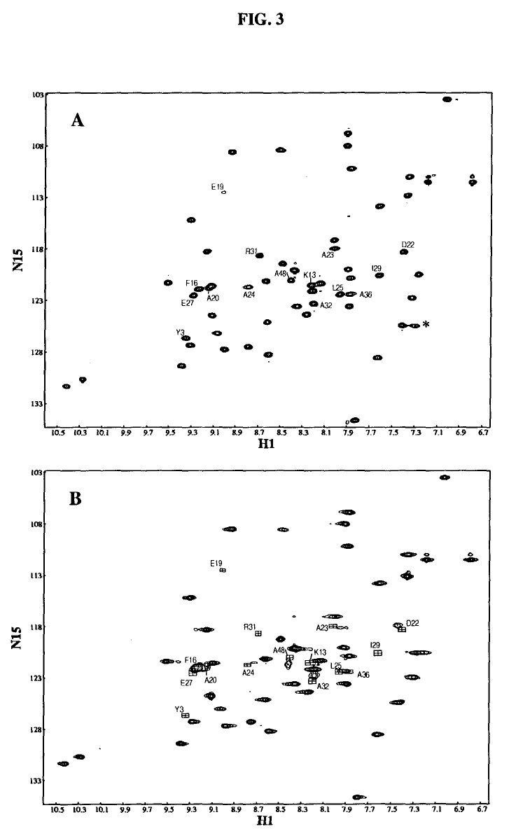

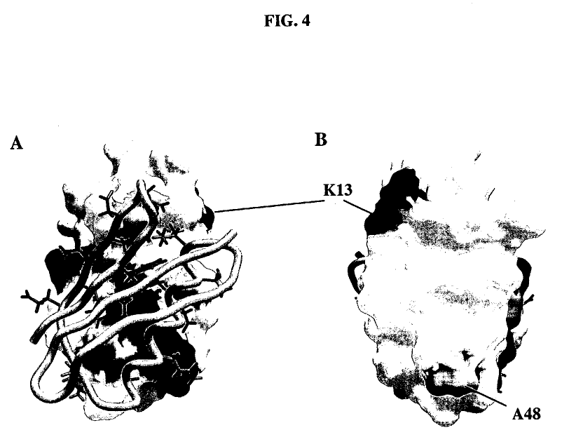

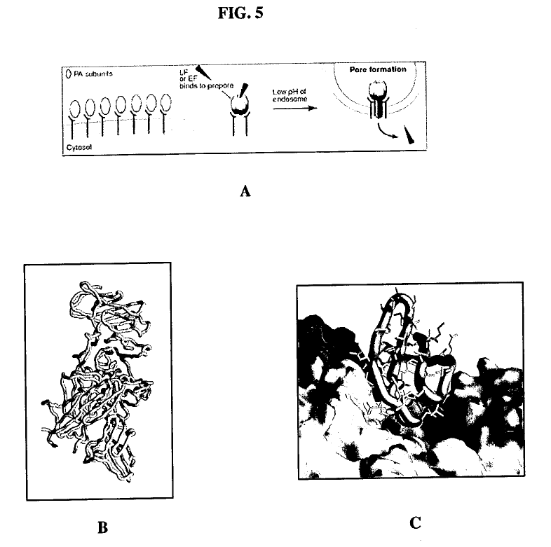

Chemical shift perturbations from preliminary 1D NMR spectra of monomer A in the presence of monomer B indicated successful complex formation, albeit with low affinity. To determine if the monomers were associating in the pre-determined target orientation, monomer A was selectively labeled with ¹⁵N, and 2D ¹⁵N-¹H HSQC spectra were collected on both free ¹⁵N-monomer-A and N-monomer-A in the presence of equimolar quantities of unlabeled monomer B (FIG. 3). Resonance assignments of ¹⁵N-monomer-A were determined via analysis of 3D-NOESY-HSQC and 3D-TOCSY-HSQC spectra.

The assigned 2D-[¹⁵N, ¹H]-HSQC peaks of free ¹⁵N-monomer-A were qualitatively compared to those of the spectra in the presence of equimolar amounts of unlabeled monomer-B. With few exceptions (Y3, K13 and A48) the peaks that exhibited chemical shift perturbations mapped in close proximity to the putative interface of the target orientation (FIG. 3, panel A).

In Vivo Genetic Screen (Step 6)

The affinity of the complex described above can be further increased upon implementation of a combinatorial process available in a commercial in vivo genetic screen (i.e., a bacterial two-hybrid screen; Stratagene). Various positions in proximity to the interface can be randomized to create a large combinatorial library of potential docking candidates (i.e., 1×10⁹). This established method also includes a genetic means to quickly determine and isolate dimeric complexes.

Additionally, specific docking and side-chain selection parameters (e.g., interfacial volume) can be systematically altered and tested to improve the computational component of the docking process. Furthermore, multidimensional NMR, as well as x-ray crystallography, can be used to solve the structures of the high affinity complexes we create.

Finally, upon successful complex formation, the redesigned dimer complex can be used as a model system to systematically mutate particular residues and assess the thermodynamic contributions of the various physical forces crucial to molecular self-assembly (i.e., the hydrophobic effect, hydrophilic effects, electrostatic interactions, hydrogen bonding and van der Waals interactions). This ultimately will provide insights and advancements in the fields of supramolecular chemistry and biological material science.

### Example 2

### Anthrax Toxin and Cancer Cell Targeting

The innovative technology disclosed herein is best described as “computer-assisted protein-based drug design.” In essence, Applicants are using today's fastest Intel CPU chips in combination with sophisticated computer algorithms and modem molecular mechanics force-fields to design antibody-like-proteins. The designed proteins are targeted to bind and inactivate proteins from pathogenic organisms or proteins associated with human diseases (e.g., antibiotic-resistant bacteria, cancer). The designed proteins surpass natural antibodies in that they are targeted to bind specific regions of pathogenic proteins and are not limited by the expression constraints inherent to in vivo systems. Applicants applied the instant invention for a protein-based antitoxin against the toxic proteins secreted by Bacillus anthracis.

The deadliest mode of Anthrax infection is the inhalation form. Upon inhalation the spores of Bacillus anthracis rapidly germinate and multiply in the warm moist milieu of the lungs. The bacteria then infiltrate the bloodstream in large numbers where they secrete deadly amounts of toxin. Although antibiotics can kill or control Anthrax expansion at this point, people infected with the inhaled form die because antibiotics do not eradicate the toxin. In contrast, the instant methods specifically target the toxin.

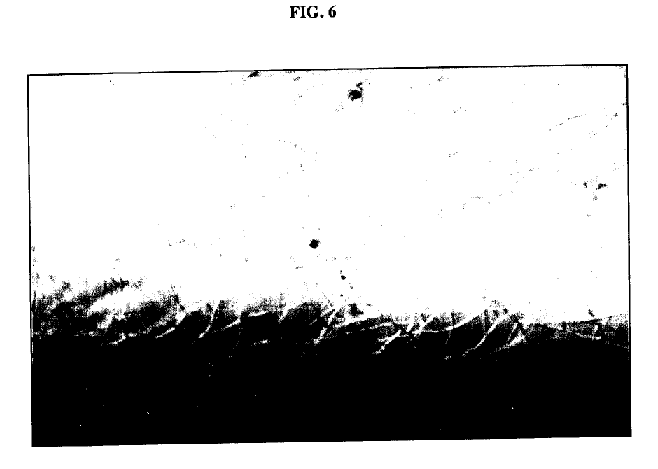

The Anthrax toxin consists of 3 proteins; protective antigen (PA), lethal factor (LF) and edema factor (EF). All 3 proteins function through very precise protein/protein interactions with each other and with native host proteins. Initially, PA (green in FIG. 5) binds to a receptor protein on the surface of host immune cells. Upon binding, a second host protein cleaves a small domain of PA which then self-assembles into a prepore complex consisting of seven PA monomers. The PA heptamer forms a complex with the two other toxin proteins (black triangles in FIG. 5) and is endocytosed into the host cell. Within the cell the low pH of the endosome induces a conformational change in PA which results in the release of LF and EF into the cytosol. Their uncontrolled interaction with endogenous host proteins leads to cell and host death. The fact that Anthrax toxicity is highly dependent on a number of precise protein/protein interactions renders PA, LF and EF excellent targets for our “computer-assisted protein-based drug design” methods. The interfacial region responsible for PA self-assembly is a good candidate for targeting. In addition, the LF and EF binding sites of PA are also preferred target sites.

Computer-Assisted Protein-based Drug Design: The computer-assisted protein-based drug design methods naturally divide into two steps. The first step entails in silico (i.e., computational) docking of a small “designer” protein to a specific site on the 3-dimensional structure of a pathogenic target protein (e.g., PA). To accomplish this, Applicants utilizes the Geometric Recognition Algorithm (GRA) which treats the two molecules as rigid bodies and uses surface complementarity as the criteria for goodness of fit. Fine-tuned matching of complementary surfaces in the target orientation is accomplished by computing the following correlation function:

$$Correlation Function \text{:} {c}_{a , β , γ} = {\sum }_{n = 1}^{N} {\sum }_{m = 1}^{N} {\sum }_{l = 1}^{N} {a}_{l , m , n} \cdot {b}_{l + a , m + β , n + γ}$$

High resolution calculations of this function are computationally intensive since they involve N³ multiplications and additions for each of the six degrees of translational and rotational freedom. Therefore, the Fourier correlation algorithm (FCA) was used, which relies on the fast Fourier transform to rapidly scan the translational space of the two rigidly rotating molecules. The application of the FCA ultimately reduces the number of translational calculations (i.e., ˜N⁶) down to the order of N³ In (N³).

Applying the FCA reduced a 25-day calculation down to 58 seconds on an SGI R10000 CPU. Currently the calculation is further reduced to ˜7.5 seconds on a 2.2 GHz Pentium® IV CPU. During this initial docking procedure the side-chains of the “designer” protein are computationally removed and its backbone is docked to the target protein with full side-chains left intact. The purpose is to not bias the interfacial space of the docked complex. The atomic coordinates corresponding to the docked complex of maximal subunit-to-subunit surface complementarity are fed directly into the second step.

Upon computational docking, the second step of our process entails the use of highly refined protein-design algorithms to computationally mutate and repack the side-chains of the “designer” (candidate) protein at the interface of the two molecules. This process is done with the ORBIT (Optimal Rotamers By Iterative Techniques) suite of protein-design algorithms. The ORBIT algorithms (which utilize modern molecular mechanics force-fields) return a mutated amino-acid sequence for the small designer protein optimized for binding the specific site on the pathogenic protein.

The mutated “designer” protein is then physically generated in the laboratory using standard tools of molecular biology and biochemistry (e.g., total gene synthesis via PCR, recombinant DNA technologies, HPLC, FPLC). Finally, Applicants assess the stability of the designed protein and the success of complex formation with standard biophysical techniques (e.g., gel filtration, ultra centrifugation, CD, NMR, X-ray crystallography).

Additionally, to greatly enhance the probability of binding success, Applicants use a powerful genetic screen (i.e., the phage display system) to experimentally explore a large portion combinatorial amino-acid sequence space (i.e., 1×10⁹).

Computational Results—Targeting Anthrax: Applicants target the surface region of the protective antigen protein (PA) that becomes buried upon self-assembly into a functional heptamer (protein-G in blue and PA in gray in FIG. 5B). Binding of our small “designer” protein (i.e., protein-G) to this interfacial region will sterically block PA complex formation, block its entry into cells and ultimately block delivery of the other toxin proteins (i.e., LF and EF) to the cytosol of the host cells.

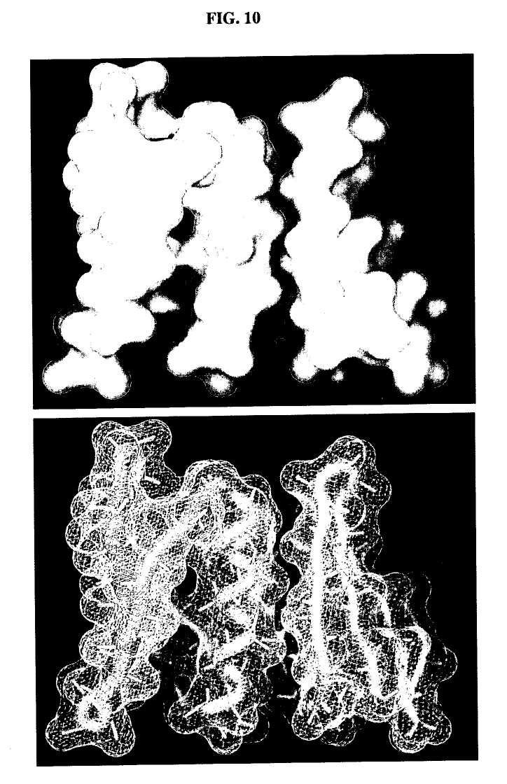

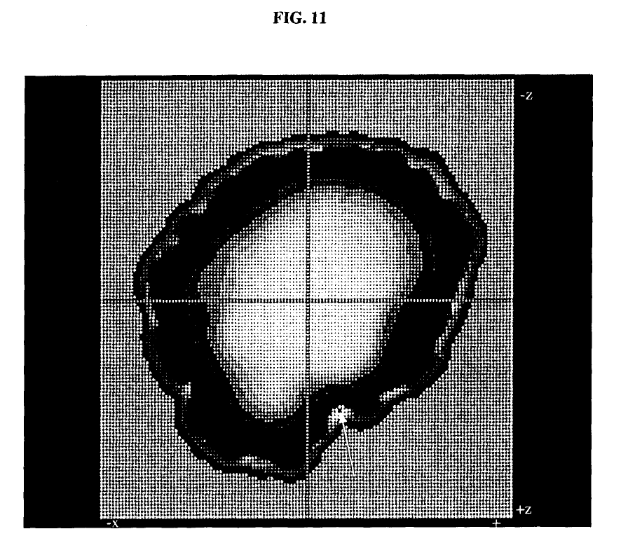

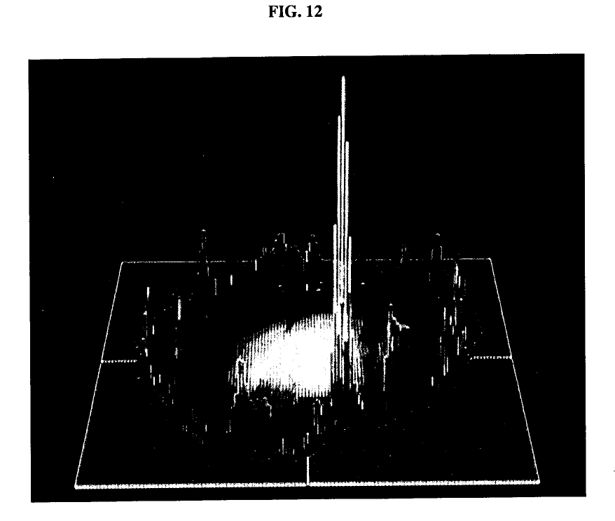

Applicants chose this initial target PA binding site based on the high degree of surface area buried in this region upon PA heptamer formation. To enhance the likelihood of docking success, Applicants increased the rotational resolution to 3° per rotation during the Geometric Recognition Algorithm (GRA). To reduce the subsequent increase in GRA calculation time, Applicants reduced N (the number of translational grid points) from 128 down to 64. To maintain an acceptable translational resolution the grid size was reduced from 64 Å down to 48 A per side of the discretized cube. The total time required to complete an entire GRA calculation (i.e., 68,921 rotational calculations) was approximately 6 hours on a 2.2 GHz Pentium® IV CPU running Linux 7.1. The discretization radius for the stationary molecule, PA, was 1.75 Å (with full side-chains) and 1.95 Å for the freely translating molecule, protein-G (with only the C_{β} atom of the side-chains). This strategy can be generally used in the instant invention to fine-tune certain parameters while keeping the overall computational time relatively constant, without dramatically sacrificing the control over other parameters. For details of the individual steo outputs, see FIGS. 10-12.

Each calculation resulted in billions of docked complexes that were rank ordered according to the measured goodness of fit (i.e., surface complementarity). To further assess which complexes were best for the next computational step (i.e., side-chain selection via the ORBIT suite of protein design algorithms), Applicants subjected the highest scoring one hundred complexes to additional analysis. For example, Applicants measured the extent of the total buried surface upon complex formation, the interfacial volume between protein-G and PA and a metric termed the gap index that corresponds to the interfacial volume divided by the total buried surface area. The gap index is an excellent measure of the degree of interdigitation of the docked interfaces.

The final choice consisted of the 36^{th} best docking score that has a total buried area of 1585.5 Å², an interfacial volume of 4691.9 Å³ and a subsequent gap index of 2.96. This complex is illustrated in FIG. 5C. In addition, this particular site was chosen based on visual inspection with the molecular graphics program GRASP. In this complex an optimal region on protein-G is juxtapositioned well with a number of side-chains of PA. This docked complex was used in the ORBIT suite of design algorithms where the interfacial residues of protein-G were subjected to computational mutagenesis. 15 residues from protein-G and 10 residues from PA change solvent accessible surface area upon complex formation.

The ORBIT design programs were run iteratively on the 15 protein-G and 10 PA residue positions. The identities of the PA residues were not allowed to vary but rotamers of these wild-type residues were examined for optimal physical chemical interactions with mutant rotamers at the 15 protein-G positions across the interface. The design programs were run approximately 7 times with different parameters varied. For example, characteristics of the substituted, mutant amino-acid types were altered (i.e., solely hydrophilic residues at some positions, both hydrophilic and hydrophobic at others) as well as important force-field parameters (i.e., salvation calculated at all positions in some cases and just on buried positions in others). The results of the above calculations resulted in two unique sequences that have 15 positions mutated relative to wild-type protein-G.

In addition to the two mutant sequences described above, Applicants used the molecular visualization program GRASP in conjunction with force-field calculations to choose positions for codon randomization at 7 key interfacial positions on protein-G. This will result in the generation of a combinatorial library with a complexity of approximately 1.28×10⁹. The library will be incorporated into a phage-display system that functions to screen for library members that bind tightly to immobilized PA (see below). These methods can also be used to target the regions of PA that have been shown to bind LF and EF. Binding of mutant protein-G variants to either of these sites will sterically block LF and EF binding and thus render Anthrax non-pathogenic.

Experimental Results—Targeting Anthrax: Applicants have obtained the gene for the protective antigen protein (PA), and have thus far successfully expressed PA in E. coli and are purifying large quantities of protein for protein-G docking analysis. Applicants have also successfully followed protocols published by John Collier's group at Harvard Medical School regarding cleavage by limited proteolysis and subsequent heptamerization of the PA protein. Applicants can use this assay to ascertain the success of the docked protein-G variants and the ability of the variants to block self-association of PA. Additionally, Applicants have expressed PA with an N-terminal (His)₆ tag and are utilizing the tag for PA purification and to ultimately immobilize PA on a nickel column. Applicants are subcloning the genes for the mutant protein-G variants into a phage-display system where phage that display the variants on their surface will be incubated with the immobilized PA bound to a nickel column. In addition, Applicants are in the process of generating a large combinatorial library (e.g., 7 positions, 20⁷ or 1.28×10⁹) of protein-G variants with 7 specific positions chosen for codon-randomization during PCR-based gene synthesis. The library of Protein-G variants will be subeloned into the phage-display system and incubated with the immobilized PA bound to the nickel column. This will enable us to select and determine the protein-G variants that bind PA with high affinity.

It is also contemplated that the PA heptamer complex of the Anthrax toxin be exploited to deliver protein-based drugs to the cytosol of diseased cells (e.g., cancerous cells). Protein-based drugs do not readily cross the cell membrane. A protein-G variant designed to bind the LF or EF site on PA can be genetically linked to a protein designed to target a cytosolic protein. Binding of the protein-G-chimer to PA and subsequent incorporation into the cell will effectively deliver the designed protein to its target. The target will be chosen such that it's inactivation upon binding will lead to the death of diseased cells (e.g., cell cycle proteins).

In addition to protein-G, Applicants have recently identified a small 60 amino-acid human protein (hyperplastic discs protein—HYD, see Reo et al., Proc Natl Acad Sci USA Apr. 10, 2001; 98(8):4414-9) that can also be used to design and target proteins from pathogenic organisms. The benefit of the HYD-protein lies in its human origin; thus there is a lower probability of a host (i.e., human) immune response against the HYD-protein itself when used to target and eradicate organisms that infect human beings.

Molecular self-assembly (e.g., protein complex formation) is the spontaneous association of molecules into stable, structurally well-defined complexes joined by noncovalent bonds. Molecular self-assembly is driven by the same forces that drive protein folding. The propensity of proteins to sequester hydrophobic residues within their core is similar to that observed at the interfaces of protein dimers. Other important interactions at protein interfaces include hydrophilic effects, electrostatic interactions, hydrogen bonding and van der Waals interactions. The methods of the instant invention are iterative by nature, and provide powerful feedback for both the ‘de novo docking’ and protein-design fields.

Additionally, the “computer-assisted protein-based drug design” methods contribute to the growing number of new medicines, antitoxins and drugs used to combat Anthrax as well as many antibiotic resistant strains of bacteria and other pathogenic organisms. Targeting the toxic Anthrax proteins also provides new tools to thwart the growing threat of international bioterrorism.

### Example 3

### A Designed Protein-Protein Interface that Blocks Fibril Formation

Protein-protein interactions underlie many of the essential functions of biological systems. As such they are widely studied and have many applications in biotechnology and medicine. Applicants have utilized the β1 domain of bacterial Protein-G as a model system. This domain is favored in protein design studies because it is only 56 amino acids in length, monomeric, and well folded. It is especially amenable to computational design studies because it lacks disulfide bonds, and its structure has been solved to high resolution. Previously, wild-type Protein G was mutated to form a binding pair of molecules termed monomer A and monomer B. In introducing the specific mutations that resulted in the binding between the two molecules, monomer A was stabilized to a hyperthermophile while monomer B was destabilized, with a Tₘ≈37° C. The binding of monomer A to monomer B was ascertained by NMR. At the concentrations required for NMR studies, monomer B alone was observed to form fibrils.

Interestingly, in the presence of monomer A no fibrils were observed. Thus, monomer A binding to monomer B is an excellent model system for the study of protein-based fibril inhibition. Protein aggregation is a problem that plagues both scientists and physicians. It can complicate protein purification by sequestering protein during recombinant expression and thus reducing yield. Fibril formation, in particular, is implicated in the pathogenesis of many diseases such as Alzheimer's, Bovine Spongiform Encephalopathy (better known as mad-cow disease), and its human variant, Creutzfeld-Jakob's Disease. Since protein fibers are resistant to proteolysis, they are difficult for cells to remove, often resulting in cell death. They are also resistant to thermal denaturation and common sterilization procedures. Thus, understanding the mechanisms that underlie the production and inhibition of protein fibers is the first step in developing therapies for these diseases.

Materials and Methods

Protein Expression and Purification

The genes for monomer A and monomer B were synthesized by PCR-based total gene synthesis. The sequences were subcloned into the pET-11m vector. All sequences were confirmed by DNA sequencing. The plasmids for monomer A and monomer B were each transformed into the BL21-(DE3) cell line purchased from Novagen. Cells were grown in standard Terrific Broth media. Protein production was induced with IPTG at an A600 of 1.2-1.5. Cells were grown for three hours post induction and then harvested. The cell pellets were frozen at −80° C. overnight. The cells were subjected to three freeze-thaw cycles. Cell pellets were thawed on ice for 30 minutes (or until visibly thawed) and then frozen for 10 minutes in a dry ice/ethanol bath. After three such cycles, the pellets were resuspended in phosphate-buffered saline (PBS), pH 7.4. The samples were centrifuged at 10,000 rpm in a Sorvall SS-34 rotor for 30 minutes. The supernatant was cut with acetonitrile to precipitate impurities, and then diluted to a final concentration of 10-15% acetonitrile, corresponding to the starting conditions for HPLC. The samples were purified on a Varian Prostar HPLC on a Microsorb C8 preparatory column, using standard reverse phase conditions and a 1% per minute gradient. Peaks corresponding to monomer A and monomer B were collected, confirmed by LCMS, and then lyophilized. The resulting powder was resuspended in ddH₂O. Monomer A required the addition of a small amount of Guanidine HCl. The proteins were concentrated and subjected to buffer-exchange through the use of Centricon filter devices (Millipore). The final concentration was determined in 8M Guanidine HCl using standard methods on a UV spectrophotometer.

Fibril Formation for Thioflavin-T Assays

Two hundred microliters of fresh, non-aggregated 1.2 mM monomer B solution were agitated in 2 ml borosilicate tubes at 37° C. and 300 rpm for 2-3 days. As negative controls, 200 μL of fresh 1.2 mM monomer A and fresh 1.2 mM Protein G wild-type were also agitated under identical conditions.

Inhibition

Equimolar quantities of monomer A and B were mixed (approximately 0.61 mM) and then agitated in a total volume of 200 μL for 2-3 days at 37° C., 300 rpm. To account for the 0.5 dilution factor 20 μl of the complex were added to the ThT assays as opposed to 10 μl for the free proteins.

Electron Microscopy

Electron microscopy imaging was performed using a Philips 410A transmission electron microscope at a 60-kV excitation voltage. 15 μl of fibril solution was air dried for 2 minutes on a 200-mesh Formvar coated copper grid. The sample was then negatively stained with 1% uranyl acetate.

Thioflavine-T Fluorescence

10 μl aliquots of the single protein solutions were added to 5 μM ThT in 0.05 M Tris HCl, 100 mM NaCl to a final volume of 1 ml. To account for the 0.5 fold dilution factor upon complex formation 20 μl aliquots of the complex protein solution was added to 5 μM ThT in 0.05 M Tris HCl, 100 mM NaCl to a final volume of 1 ml. Fluorescence spectra were recorded on a Spex FluoroMax spectrofluorometer with an excitation wavelength of 450 nm, scanning emission from 470-560 nm.

Results and Discussion

Transmission Electron Microscopy

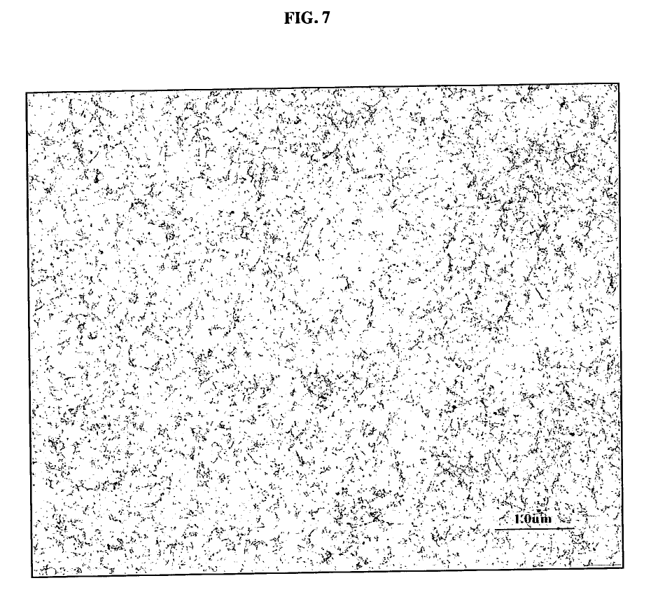

FIG. 7 shows the transmission electron micrograph of the agitated monomer B sample. The image clearly shows the presence of protein fibrils.

Thioflavine T fluorescence

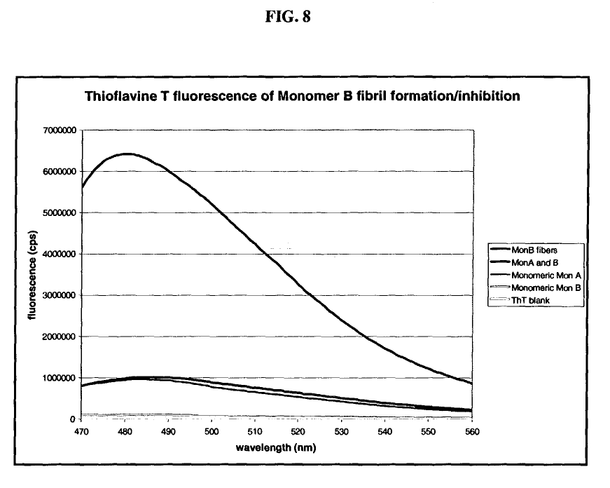

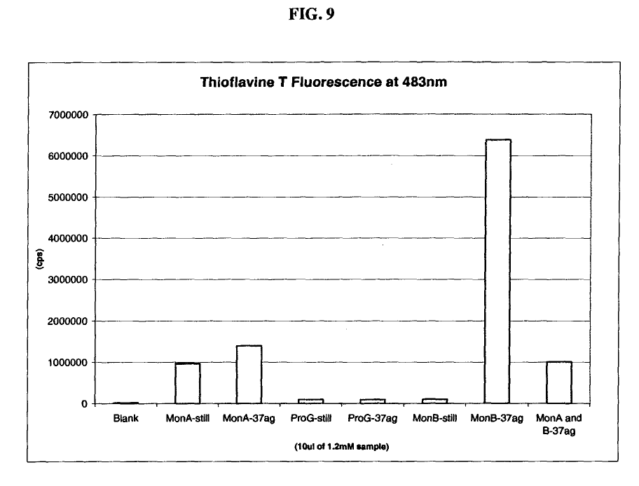

Upon agitation at 37° C., monomer B shows a six-fold increase in Thioflavine-T fluorescence, indicating the formation of amyloid type fibrils (FIG. 8). The increase in Thioflavine-T fluorescence indicates the presence of intermolecular beta-sheet structures as found in fibrillated proteins, presumably through direct interactions between Thioflavine-T and the beta-sheet of the amyloid fibril.

The thioflavine-T fluorescence emission spectrum for unagitated monomer B (light blue curve) indicates no relative increase in fluorescence when compared to the scan of just thioflavin-T (yellow curve). In stark contrast, the scan for agitated monomer-B (dark blue curve) increases approximately 6 fold over unagitated monomer B. For monomer A there is a minor increase in fluorescence for the agitated and non-agitated samples. We attribute this increase to the fact that there was a small amount of precipitate observed in the solution of monomer A upon resuspension following lyophilization. Fiber inhibition is evidenced by the lack of increase in fluorescence for the agitated sample of equimolar concentrations of monomer A and monomer B. This suggests that monomer A is blocking the formation of monomer B fibrils.

### CONCLUSIONS

The designed interaction between monomer A and B is sufficiently strong enough to block the formation of monomer B fibrils. Understanding the mechanism of this block in fibril formation will help elucidate the mechanism of fibril formation. This is the first step in the development of designed protein-based pharmaceuticals for these diseases.

### REFERENCES

1. Bracewell, R.N. (1990). Numerical Transformations. Science, 248: 697-704.

2. Dahiyat, B. I. and Mayo, S. L. (1997). De Novo Protein Design: Fully Automated Sequence Selection. Science 278: 82-87.

3. Gabb, H. A., Jackson, R. M., Sternberg, M. J. E. (1997). Modeling Protein Docking using Shape Complementarity, Electrostatics and Biochemical Information. Journal of Molecular Biology 272: 106-120.

4. Katchalski-Katzir, E., Shariv, I., Eisenstein, M., Friesem, A. A., Aflalo, C., Vakser, I. A. (1992). Molecular surface recognition: Determination of geometric fit between proteins and their ligands by correlation techniques. PNAS 89: 2195-2199.

5. Press, W. H., Teukolsky, S. A., Vetterling, W. T., Flannery, B. P. (1986). Numerical Recipes in Fortran, Cambridge University Press, Cambridge.

6. Street, A. G. and Mayo, S. L (1999). Computational Protein Design. Structure 7(5): 105-109.

7. Gallagher, T., Alexander, P., Bryan, P. Gilliland, G. L. (1994). Two crystal structures of the β1 immunoglobulin-binding domain of streptococcal protein G and comparison with NMR. Biochemistry 33: 4721-4729.

8. Gordon, D. B., Marshall, S. A., and Mayo, S. L. (1999). Energy functions for protein design. Current Opinions in Structural Biology 9(4): 509-513.

9. Gronenborn, A. M. et al., (1991). A novel, highly stable fold of the immunoglobulin binding domain of streptococcal protein G. Science 253: 657-661.

10. Jones, S. and Thornton, J. M. (1997). Prediction of Protein-Protein Interaction Sites using Patch Analysis. Journal of Molecular Biology 272: 133-143.

11. Malakauskas, S. M. and Mayo, S. L. (1998). Design, structure and stability of a hyperthermophilic protein variant. Nature Structural Biology 5(6): 470-475

12. Nicholls, A., Sharp, K. A., Honig, B. (1991). Protein folding and association: insights from the interfacial and thermodynamic properties of hydrocarbons. Proteins 11(4): 281-296.

13. Press, W. H., Teukolsky, S. A., Vetterling, W. T., Flannery, B. P. (1986). Numerical Recipes in Fortran, Cambridge University Press, Cambridge.

The practice of the present invention will employ, unless otherwise indicated, conventional techniques of molecular biology, cell biology, cell culture, microbiology and recombinant DNA, which are within the skill of the art. Such techniques are explained fully in the literature. See, for example, Molecular Cloning: A Laboratory Manual, 2^{nd} Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al.; U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

The contents of all cited references (including literature references, issued patents, published patent applications as cited throughout this application) are hereby expressly incorporated by reference.

Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

What is claimed is:

1. A method for modifying a candidate polypeptide sequence to alter interaction with a target biopolymer, comprising:
(a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) identifying, by surface-to-surface geometric fitting, a model of a complex between said target biopolymer model and said candidate polypeptide model that has at least a predefined degree of surface shape complementarity;
(c) identifying amino acid residues in said candidate polypeptide with unfavorable interactions with said target biopolymer in said complex as varying residues;
(d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

2. The method of 1, wherein said atomic coordinate model of said candidate polypeptide includes coordinates for only backbone atoms but not C_{β} atoms of said reference amino acid sequence.

3. The method of claim 1, wherein said atomic coordinate model of said candidate polypeptide and said atomic coordinate model of said target biopolymer are obtained from known crystallographic or NMR structures.

4. The method of claim 1, wherein said atomic coordinate model of said candidate polypeptide and said atomic coordinate model of said target biopolymer are established by homology modeling based on a known crystallographic or NMR structure of a homolog of said target biopolymer or a homolog of said candidate polypeptide.

5. The method of claim 4, wherein said homolog is at least about 70% identical to said candidate polypeptide in the binding region; or at least about 70% identical to said target biopolymer, wherein said target biopolymer is a polypeptide.

6. The method of claim 1, wherein said target biopolymer is a lipid, a vitamin co-factor, or a steroid.

7. The method of claim 1, wherein said target biopolymer is a protein, a polynucleotide, or a polysaccharide.

8. The method of claim 1, wherein said target biopolymer is a protein, and wherein said docking surface is an atomic coordinate model of said target protein, which model includes coordinates for at least backbone atoms of exposed surface residues.

9. The method of claim 8, wherein said target protein model additionally include coordinates for C_{β} atoms of exposed surface residues.

10. The method of claim 9, wherein said target protein model additionally include coordinates for more than C_{β} atoms of exposed surface residues.

11. The method of claim 8, wherein said target protein model additionally include coordinates for at least backbone atoms of non-surface residues.

12. The method of claim 1, wherein said surface-to-surface geometric fitting is identified in step (b) by:
(A) computationally projecting said atomic coordinate model of said candidate polypeptide and said target biopolymer onto a three-dimensional grid, and fixing the atomic coordinate model of said target biopolymer in a pre-defined target orientation;
(B) assessing intermolecular surface shape complementarity between said candidate polypeptide and said target biopolymer as a function of their relative translational and rotational positions, by rotating and translating the atomic coordinate model of said candidate polypeptide;
(C) identifying the optimal atomic coordinate model associated with the best intermolecular surface shape complementarity; and,
(D) combining the optimal atomic coordinate models of the docked said candidate polypeptide and said target biopolymer as the atomic coordinate model of said complex.

13. The method of claim 1, wherein step (c) is effected by:
(A) classifying residues of said candidate polypeptide as core, boundary, or surface residues, first in the context of the undocked form and then in the context of said complex; and,
(B) identifying residues which either change classification upon complex formation, or are in close proximity to form favorable intermolecular interactions as said varying residues.

14. The method of claim 13, wherein said target biopolymer is a protein.

15. The method of claim 1, wherein step (d) is effected by:
(A) providing the coordinates for a plurality of potential rotamers resulting from varying torsional angles for side-chains of each of said varying residues identified in (c), wherein said plurality of potential rotamers for at least one of said varying residues have rotamers selected from each of at least two different amino acid side-chains; and
(B) modeling interactions of each of said rotamers with all or part of the remaining structure of said complex to generate a set of globally optimized protein sequences.

16. The method of claim 12, wherein said three-dimensional grid comprises N×N×N nodes.

17. The method of claim 16, wherein N is 128.

18. The method of claim 12, wherein the size of said grid is the sum of the radii of said candidate polypeptide and said target biopolymer plus 1 Å.

19. The method of claim 12, wherein the size of said grid is the sum of the radii of said candidate polypeptide and a potential candidate-polypeptide-binding region of said target biopolymer plus 1 Å.

20. The method of claim 12, wherein said surface-to-surface geometric fitting is identified by a geometric recognition algorithm (GRA).

21. The method of claim 20, wherein said GRA further incorporates a Fourier Correlation Algorithm (FCA).

22. The method of claim 21, wherein said FCA comprises discrete fast Fourier transformation (DFT) of said candidate polypeptide and said target biopolymer.

23. The method of claim 20 or 21, further comprising measuring electrostatic complementarity by Fourier correlation.

24. The method of claim 20 or 21, further comprising distance filtering.

25. The method of claim 20 or 21, further comprising local refinement of predicted geometries.

26. The method of claim 20 or 21, wherein the method is repeated more than once with successively more fine-tuned parameters for assessing intermolecular surface-to-surface geometric fitting.

27. The method of claim 20 or 21, further comprising one or more of: measuring electrostatic complementarity by Fourier correlation, distance filtering, or local refinement of predicted geometries.

28. The method of claim 15, wherein said plurality of potential rotamers for said varying residues are from a backbone-dependent rotamer library.

29. The method of claim 15, wherein said torsional angles for side-chains of each of said varying residues are changed by varying both the χ1 and χ2 torsional angles by ±20 degrees, in increment of 5 degrees, from the values of said varying residues in the context of the undocked candidate polypeptide.

30. The method of claim 15, further comprising a Dead-End Elimination (DEE) computation in step (B).

31. The method of claim 30, wherein said DEE computation is selected from original DEE or Goldstein DEE.

32. The method of claim 15, wherein step (B) further includes the use of at least one scoring function.

33. The method of claim 32, wherein said scoring function is selected from: van der Waals potential scoring function, hydrogen bond potential scoring function, atomic solvation scoring function, electrostatic scoring function or secondary structure propensity scoring function.

34. The method of claim 15, wherein step (B) further includes the use of at least two scoring functions.

35. The method of claim 15, wherein step (B) further includes the use of at least three scoring functions.

36. The method of claim 15, wherein step (B) further includes the use of at least four scoring functions.

37. The method of claim 33, wherein said atomic solvation scoring function includes a scaling factor that compensates for over-counting.

38. The method of claim 15, further comprising generating a rank ordered list of additional optimal sequences from said globally optimal protein sequence.

39. The method of claim 38, wherein said generating includes the use of a Monte Carlo search.

40. The method of claim 38, further comprising testing some or all of said protein sequences from said ordered list to produce potential energy test results.

41. The method of claim 40, further comprising analyzing the correspondence between said potential energy test results and theoretical potential energy data.

42. The method of claim 15, wherein said varying residue identified in step (c) are residues re-classified as core residues upon complex formation, and wherein said plurality of potential rotamers for said varying residues have rotamers selected from each of at least two different hydrophobic amino acid side-chains.

43. The method of claim 42, wherein said at least two hydrophobic amino acids are selected from: alanine, valine, isoleucine, leucine, phenylalanine, tyrosine, tryptophan, or methionine.

44. The method of claim 15, wherein said varying residue identified in step (c) are residues re-classified from surface to boundary residues upon complex formation, and wherein said plurality of potential rotamers for said varying residues have rotamers selected from each of at least two different hydrophilie amino acid side-chains.

45. The method of claim 44, wherein said at least two hydrophilic amino acids are selected from: alanine, serine, threonine, aspartic acid, asparagine, glutamine, glutamic acid, arginine, lysine or histidine.

46. The method of claim 15, wherein said varying residue identified in step (c) are residues re-classified as boundary residues upon complex formation, and wherein said plurality of potential rotamers for said varying residues have rotamers selected from each of at least two different amino acid side-chains selected from: alanine, serine, threonine, aspartic acid, asparagine, glutamine, glutamic acid, arginine, lysine histidine, valine, isoleucine, leucine, phenylalanine, tyrosine, tryptophan, or methionine.

47. The method of claim 1, further comprising generating said target biopolymer, and one or more modified versions of said candidate polypeptide with said mutations of said varying residues that form more favorable interactions with said target biopolymer model, and assessing the degree of complex formation.

48. The method of claim 47, wherein said degree of complex formation is assessed in vitro or in vivo.

49. The method of claim 1, further comprising verifying, by solving the three-dimensional structure(s) of, one or more modified versions of said candidate polypeptide with said mutations of said varying residues that form more favorable interactions with said target biopolymer model.

50. The method of claim 1, wherein said candidate polypeptide is an antibody or functional fragment thereof.

51. The method of claim 1, wherein said target biopolymer is an enzyme, and said candidate polypeptide is an inhibitor of said enzyme.

52. The method of claim 1, wherein said target biopolymer is a target protein, wherein step (c) further includes identifying amino acid residues in said target protein with unfavorable interactions with said candidate polypeptide in said complex as varying residues, and wherein step (d) is additionally effected by identifying mutations of said varying residues of said target protein that form more favorable interactions with said candidate polypeptide.

53. The method of claim 52, wherein said target protein and said candidate polypeptide are identical.

54. A complex comprising a target biopolymer and a redesigned candidate polypeptide generated by the method of claim 1.

55. A nucleic acid sequence encoding a target polypeptide and a nucleic acid sequence encoding a redesigned candidate polypeptide according to claim 54.

56. An expression vector comprising the nucleic acid sequences of claim 55.

57. A host cell comprising the nucleic acid sequences of claim 55.

58. An apparatus for redesigning a candidate polypeptide sequence to alter interaction with a target biopolymer, said apparatus comprising:
(a) means for providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) means for identifying, by surface-to-surface geometric fitting, a model of a complex between said target biopolymer model and said candidate polypeptide model that has at least a predefined degree of surface shape complementarity;
(c) means for identifying amino acid residues in said candidate polypeptide with unfavorable interactions with said target biopolymer in said complex as varying residues;
(d) means for generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

59. A computer system for use in redesigning a candidate polypeptide sequence to alter interaction with a target biopolymer, said computer system comprising computer instructions for:
(a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) identifying, by surface-to-surface geometric fitting, a model of a complex between said target biopolymer model and said candidate polypeptide model that has at least a predefined degree of surface shape complementarity;
(c) identifying amino acid residues in said candidate polypeptide with unfavorable interactions with said target biopolymer in said complex as varying residues;
(d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

60. A computer-readable medium storing a computer program executable by a plurality of server computers, the computer program comprising computer instructions for:
(a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) identifying, by surface-to-surface geometric fitting, a model of a complex between said target biopolymer model and said candidate polypeptide model that has at least a predefined degree of surface shape complementarity;
(c) identifying amino acid residues in said candidate polypeptide with unfavorable interactions with said target biopolymer in said complex as varying residues;
(d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

61. A computer data signal embodied in a carrier wave, comprising computer instructions for:
(a) providing (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) identifying, by surface-to-surface geometric fitting, a model of a complex between said target biopolymer model and said candidate polypeptide model that has at least a predefined degree of surface shape complementarity;
(c) identifying amino acid residues in said candidate polypeptide with unfavorable interactions with said target biopolymer in said complex as varying residues;
(d) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

62. An apparatus comprising a computer readable storage medium having instructions stored thereon for:
(a) accessing a datafile representative of (i) an atomic coordinate model of a candidate polypeptide having a reference amino acid sequence, which model includes coordinates for backbone atoms and coordinates for no more than C_{β} atoms of amino acid side-chains of said reference amino acid sequence, and (ii) an atomic coordinate model for at least a docking surface of said target biopolymer;
(b) accessing a datafile representative of the atomic coordinates for a plurality of different rotamers of amino acids resulting from varying torsional angles;
(c) a set of modeling routines for:
(1) identifying surface-to-surface geometric fitting by docking said candidate polypeptide and said target biopolymer to form a complex with a predefined degree of surface shape complementarity between said candidate polypeptide and said target biopolymer;
(2) generating one or more model(s) of said complex in which said candidate polypeptide model includes atomic coordinates of more than the C_{β} atoms of said varying residue side-chains, and identifying mutations of said varying residues that form more favorable interactions with said target biopolymer model.

63. A method for conducting a biotechnology business comprising:
(1) redesigning, according to the method of claim 1, a candidate polypeptide sequence to alter interaction with a target biopolymer;
(2) producing said candidate polypeptide.

64. The business method of claim 63, further comprising the step of providing a packaged pharmaceutical including said candidate polypeptide and/or said target biopolymer, and instructions and/or a label describing how to administer said redesigned candidate polypeptide.

65. A method for inhibiting the binding of a candidate polypeptide to a target biopolymer, comprising:
(a) redesigning, using the method of claim 1, a set of globally optimized complexes comprising a redesigned candidate polypeptide and said target biopolymer;
(b) obtaining an inhibitory polypeptide sequence comprising the interfacial residue sequences of said redesigned candidate polypeptide;
(c) providing said inhibitory polypeptide sequence to a mixture containing said candidate polypeptide and said target biopolymer, thereby inhibiting the binding of said candidate polypeptide to said target biopolymer.
